SEQUENCE LISTING (40 sequences)

SEQ ID NO: 1 - protein, 735 aa, Adeno-associated dependoparvovirus A [sequence omitted]

SEQ ID NO: 2 - protein, 736 aa, Adeno-associated dependoparvovirus A
  source 1..736: note = AAV9 capsid (VP1 protein)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 12 aa, Synthetic construct
  source 1..12: note = AAV2 substitution consensus
  VARIANT 4: note = X is K or R
  VARIANT 5: note = X is K or R
  VARIANT 6: note = X is G or D
  VARIANT 7: note = X is D or V
  VARIANT 8: note = X is S or D
  VARIANT 9: note = X is W or F
  VARIANT 10: note = X is S or D
  VARIANT 11: note = X is E or K
GQRXXXXXXXXA

SEQ ID NO: 4 - protein, 12 aa, Synthetic construct
  source 1..12: note = Pep2hSC1 substitution
GQRKRGDSWSEA

SEQ ID NO: 5 - protein, 12 aa, Synthetic construct
  source 1..12: note = Pep2hSC2 substitution
GQRRKDVDFDKA

SEQ ID NO: 6 - protein, 742 aa, Synthetic construct
  source 1..742: note = Pep2hSC1 capsid polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRKRGDSWSEAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - protein, 742 aa, Synthetic construct
  source 1..742: note = Pep2hSC2 capsid polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRRKDVDFDKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - skipped

SEQ ID NO: 9 - protein, 12 aa, Synthetic construct
  source 1..12: note = AAV9 substitution consensus sequence 2
  VARIANT 4: note = X is A or G
  VARIANT 8: note = X is V or M
  VARIANT 9: note = X is R, A or L
  VARIANT 10: note = V or G
  VARIANT 11: note = X is Q, E or K
GQSXRGDXXXXA

SEQ ID NO: 10 - protein, 12 aa, Synthetic construct
  source 1..12: note = Pep9hSC1 substitution
GQSARGDVRVQA

SEQ ID NO: 11 - protein, 12 aa, Synthetic construct
  source 1..12: note = Pep9hSC2 substitution
GQRGRGDVAGEA

SEQ ID NO: 12 - protein, 12 aa, Synthetic construct
  source 1..12: note = Pep9hSC5 substitution
GQSRADRPPPQA

SEQ ID NO: 13 - protein, 12 aa, Synthetic construct
  source 1..12: note = Pep9hSC9 substitution
GQSARGDMLGKA

SEQ ID NO: 14 - protein, 743 aa, Synthetic construct
  source 1..743: note = Pep9hSC1 capsid polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSARGDVRVQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 743 aa, Synthetic construct
  source 1..743: note = Pep9hSC2 capsid polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQRGRGDVAGEAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 743 aa, Synthetic construct
  source 1..743: note = Pep9hSC5 capsid polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSRADRPPPQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 743 aa, Synthetic construct
  source 1..743: note = Pep9hSC9 capsid polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSARGDMLGKAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 18 - protein, 736 aa, Synthetic construct
  source 1..736: note = Shuffle32 capsid polypeptide
MAADGYLPDWLEDNLSEGIREWWDLKPGAPKPKANQQKQDDGRGLVLPGYKYLGPFNGLDKGEPVNEADAAALEHDKAYDQQLKAGDNPYLRYNHADAEFQERLQEDTSFGGNLGRAVFQAKKRLLEPLGLVEEAAKTAPAKKRPVEPSPQRSPDSSTGIGKKGQQPARKRLNFGQTGDSESVPDPQPLGEPPATPAAVGPTTMASGGGAPMADNNEGADGVGNASGNWHCDSTWLGDRVITTSTRTWALPTYNNHLYKQISSQSGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYYKSTNVDFAVDSQGVYSEPRPIGTRYLTRNL

SEQ ID NO: 19 - protein, 736 aa, Synthetic construct
  source 1..736: note = Shuffle35 capsid polypeptide
MAADGYLPDWLEDNLSEGIRQWWKLKPGPPPPKPAERHKDDSRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSAGIGKSGAQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNGGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQIMIKNTPVPANPSTTFSAAKFASFITQYSTGQVSVEIEWELQKENSKRWNPEIQYTSNYYKSTSVDFAVNTEGVYSEPRPIGTRYLTRNL

SEQ ID NO: 20 - DNA, 2229 bp, Synthetic construct
  source 1..2229: mol_type = other DNA; note = Pep2hSC1 capsid nucleotide sequence
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggaagcggggtgattcgtggtctgaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 21 - DNA, 2229 bp, Synthetic construct
  source 1..2229: mol_type = other DNA; note = Pep2hSC2 capsid nucleotide sequence
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggaggaaggatgttgattttgataaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 22 - DNA, 2232 bp, Synthetic construct
  source 1..2232: mol_type = other DNA; note = Pep9hSC1 capsid nucleotide sequence
atggctgccgatggttatcttccagattggctcgaggacaacctctctgaaggcattcgcgagtggtgggctttgaaacctggagccccgcaacccaaagccaaccagcaacatcaggacaacgctcggggtcttgtgcttcctggttacaagtacctcggacccggcaacggactcgacaagggggagcccgtcaacgcggcggacgcagcggccctcgagcacgacaaggcctacgaccagcagctcaaggcgggtgacaacccgtacctcaagtacaaccacgccgacgccgagtttcaggagcgtctgaaagaagatacgtcttttgggggcaacctcgggcgagcagtcttccaggccaagaagaggcttctcgaacctcttggtctggttgaggaagcggctaagacggctcctggaaagaagcgtccggtagagcagtctccacaggagccagactcctccgcgggcatcggcaagtcgggcgcacagcccgctaaaaagagactcaattttggtcagactggcgacacagagtcagtccccgaccctcaacctatcggagaacctccagcagccccctctggtgtgggatctcttacaatggcttcaggcggtggcgcaccagtcgcagacaataacgaaggcgccgacggagtgggtagttcctcgggaaattggcattgcgattcccaatggctgggcgacagagtcatcaccaccagcacccgaacctgggccctgcccacctacaacaaccacctctacaagcaaatctccaacagtacatcaggaggaagcagcaacgacaacgcctacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacggacaataacggcgtcaagaccatcgccaataaccttaccagcacggttcaagtgtttacggactcggactaccagctcccgtacgtcctcggctctgcgcacgagggctgcctccctccgttcccggcggacgtcttcatgattccgcagtacggctacctgacgctcaacgatggcagccaggccgtgggacgctcatccttttactgcctggaatacttcccttcgcagatgctgagaacgggcaacaactttcagttcagctacgagtttgagaacgtgcctttccacagcagctacgctcacagccagagcctggaccggctgatgaatcctctcatcgaccagtacctgtactacctgagcaagactattaatggatctggacagaatcaacagaccttgaaatttagcgtggccggacctagtaacatggccgttcagggaagaaactacatacctggaccctcataccggcagcagcgcgtttcaacgacagtgacacaaaacaacaacagcgaatttgcctggcctggtgcttcatcttgggctctcaatggccgtaattctctgatgaatcctggcccggctatggcctcacacaaagaaggagaagaccgtttctttcccctgagcggttctctcatttttggcaaacaaggcacaggaagagacaacgtggatgcggacaaggtcatgattaccaacgaagaggaaattaaaaccactaaccctgtggccacggagtcctatgggcaggtggcaaccaaccaccagggccagagtgcgaggggcgacgtgcgcgtccaggcggcccaaaccggatgggtccaaaaccagggaatcttacctggcatggtgtggcaagacagagacgtgtacctgcagggtcccatttgggccaagattcctcacacggatggaaactttcacccgtctccgctgatgggcggctttggaatgaaacacccgcctcctcagatcctcatcaaaaacacgcctgtacctgcggatcctccgaccgccttcaacaaggacaagctgaactcattcatcacccagtactccactggacaggtcagcgtggagattgagtgggagctgcagaaggaaaacagcaagcgctggaatcccgagatccagtacacatccaactactacaaatctaacaatgtggaatttgctgtgaatactgaaggagtgtatagtgagcctcgccccattggcacccgttacctcacccgtaatctgtaa

SEQ ID NO: 23 - DNA, 2232 bp, Synthetic construct
  source 1..2232: mol_type = other DNA; note = Pep9hSC2 capsid nucleotide sequence
atggctgccgatggttatcttccagattggctcgaggacaacctctctgaaggcattcgcgagtggtgggctttgaaacctggagccccgcaacccaaagccaaccagcaacatcaggacaacgctcggggtcttgtgcttcctggttacaagtacctcggacccggcaacggactcgacaagggggagcccgtcaacgcggcggacgcagcggccctcgagcacgacaaggcctacgaccagcagctcaaggcgggtgacaacccgtacctcaagtacaaccacgccgacgccgagtttcaggagcgtctgaaagaagatacgtcttttgggggcaacctcgggcgagcagtcttccaggccaagaagaggcttctcgaacctcttggtctggttgaggaagcggctaagacggctcctggaaagaagcgtccggtagagcagtctccacaggagccagactcctccgcgggcatcggcaagtcgggcgcacagcccgctaaaaagagactcaattttggtcagactggcgacacagagtcagtccccgaccctcaacctatcggagaacctccagcagccccctctggtgtgggatctcttacaatggcttcaggcggtggcgcaccagtcgcagacaataacgaaggcgccgacggagtgggtagttcctcgggaaattggcattgcgattcccaatggctgggcgacagagtcatcaccaccagcacccgaacctgggccctgcccacctacaacaaccacctctacaagcaaatctccaacagtacatcaggaggaagcagcaacgacaacgcctacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacggacaataacggcgtcaagaccatcgccaataaccttaccagcacggttcaagtgtttacggactcggactaccagctcccgtacgtcctcggctctgcgcacgagggctgcctccctccgttcccggcggacgtcttcatgattccgcagtacggctacctgacgctcaacgatggcagccaggccgtgggacgctcatccttttactgcctggaatacttcccttcgcagatgctgagaacgggcaacaactttcagttcagctacgagtttgagaacgtgcctttccacagcagctacgctcacagccagagcctggaccggctgatgaatcctctcatcgaccagtacctgtactacctgagcaagactattaatggatctggacagaatcaacagaccttgaaatttagcgtggccggacctagtaacatggccgttcagggaagaaactacatacctggaccctcataccggcagcagcgcgtttcaacgacagtgacacaaaacaacaacagcgaatttgcctggcctggtgcttcatcttgggctctcaatggccgtaattctctgatgaatcctggcccggctatggcctcacacaaagaaggagaagaccgtttctttcccctgagcggttctctcatttttggcaaacaaggcacaggaagagacaacgtggatgcggacaaggtcatgattaccaacgaagaggaaattaaaaccactaaccctgtggccacggagtcctatgggcaggtggcaaccaaccaccagggccagaggggtcggggtgatgtggctggggaggcggcccaaaccggatgggtccaaaaccagggaatcttacctggcatggtgtggcaagacagagacgtgtacctgcagggtcccatttgggccaagattcctcacacggatggaaactttcacccgtctccgctgatgggcggctttggaatgaaacacccgcctcctcagatcctcatcaaaaacacgcctgtacctgcggatcctccgaccgccttcaacaaggacaagctgaactcattcatcacccagtactccactggacaggtcagcgtggagattgagtgggagctgcagaaggaaaacagcaagcgctggaatcccgagatccagtacacatccaactactacaaatctaacaatgtggaatttgctgtgaatactgaaggagtgtatagtgagcctcgccccattggcacccgttacctcacccgtaatctgtaa

SEQ ID NO: 24 - DNA, 2232 bp, Synthetic construct
  source 1..2232: mol_type = other DNA; note = Pep9hSC5 capsid nucleotide sequence
atggctgccgatggttatcttccagattggctcgaggacaacctctctgaaggcattcgcgagtggtgggctttgaaacctggagccccgcaacccaaagccaaccagcaacatcaggacaacgctcggggtcttgtgcttcctggttacaagtacctcggacccggcaacggactcgacaagggggagcccgtcaacgcggcggacgcagcggccctcgagcacgacaaggcctacgaccagcagctcaaggcgggtgacaacccgtacctcaagtacaaccacgccgacgccgagtttcaggagcgtctgaaagaagatacgtcttttgggggcaacctcgggcgagcagtcttccaggccaagaagaggcttctcgaacctcttggtctggttgaggaagcggctaagacggctcctggaaagaagcgtccggtagagcagtctccacaggagccagactcctccgcgggcatcggcaagtcgggcgcacagcccgctaaaaagagactcaattttggtcagactggcgacacagagtcagtccccgaccctcaacctatcggagaacctccagcagccccctctggtgtgggatctcttacaatggcttcaggcggtggcgcaccagtcgcagacaataacgaaggcgccgacggagtgggtagttcctcgggaaattggcattgcgattcccaatggctgggcgacagagtcatcaccaccagcacccgaacctgggccctgcccacctacaacaaccacctctacaagcaaatctccaacagtacatcaggaggaagcagcaacgacaacgcctacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacggacaataacggcgtcaagaccatcgccaataaccttaccagcacggttcaagtgtttacggactcggactaccagctcccgtacgtcctcggctctgcgcacgagggctgcctccctccgttcccggcggacgtcttcatgattccgcagtacggctacctgacgctcaacgatggcagccaggccgtgggacgctcatccttttactgcctggaatacttcccttcgcagatgctgagaacgggcaacaactttcagttcagctacgagtttgagaacgtgcctttccacagcagctacgctcacagccagagcctggaccggctgatgaatcctctcatcgaccagtacctgtactacctgagcaagactattaatggatctggacagaatcaacagaccttgaaatttagcgtggccggacctagtaacatggccgttcagggaagaaactacatacctggaccctcataccggcagcagcgcgtttcaacgacagtgacacaaaacaacaacagcgaatttgcctggcctggtgcttcatcttgggctctcaatggccgtaattctctgatgaatcctggcccggctatggcctcacacaaagaaggagaagaccgtttctttcccctgagcggttctctcatttttggcaaacaaggcacaggaagagacaacgtggatgcggacaaggtcatgattaccaacgaagaggaaattaaaaccactaaccctgtggccacggagtcctatgggcaggtggcaaccaaccaccagggccagagtcgtgctgatcgtcctcctcctcaggcggcccaaaccggatgggtccaaaaccagggaatcttacctggcatggtgtggcaagacagagacgtgtacctgcagggtcccatttgggccaagattcctcacacggatggaaactttcacccgtctccgctgatgggcggctttggaatgaaacacccgcctcctcagatcctcatcaaaaacacgcctgtacctgcggatcctccgaccgccttcaacaaggacaagctgaactcattcatcacccagtactccactggacaggtcagcgtggagattgagtgggagctgcagaaggaaaacagcaagcgctggaatcccgagatccagtacacatccaactactacaaatctaacaatgtggaatttgctgtgaatactgaaggagtgtatagtgagcctcgccccattggcacccgttacctcacccgtaatctgtaa

SEQ ID NO: 25 - DNA, 2232 bp, Synthetic construct
  source 1..2232: mol_type = other DNA; note = Pep9hSC9 capsid nucleotide sequence
atggctgccgatggttatcttccagattggctcgaggacaacctctctgaaggcattcgcgagtggtgggctttgaaacctggagccccgcaacccaaagccaaccagcaacatcaggacaacgctcggggtcttgtgcttcctggttacaagtacctcggacccggcaacggactcgacaagggggagcccgtcaacgcggcggacgcagcggccctcgagcacgacaaggcctacgaccagcagctcaaggcgggtgacaacccgtacctcaagtacaaccacgccgacgccgagtttcaggagcgtctgaaagaagatacgtcttttgggggcaacctcgggcgagcagtcttccaggccaagaagaggcttctcgaacctcttggtctggttgaggaagcggctaagacggctcctggaaagaagcgtccggtagagcagtctccacaggagccagactcctccgcgggcatcggcaagtcgggcgcacagcccgctaaaaagagactcaattttggtcagactggcgacacagagtcagtccccgaccctcaacctatcggagaacctccagcagccccctctggtgtgggatctcttacaatggcttcaggcggtggcgcaccagtcgcagacaataacgaaggcgccgacggagtgggtagttcctcgggaaattggcattgcgattcccaatggctgggcgacagagtcatcaccaccagcacccgaacctgggccctgcccacctacaacaaccacctctacaagcaaatctccaacagtacatcaggaggaagcagcaacgacaacgcctacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacggacaataacggcgtcaagaccatcgccaataaccttaccagcacggttcaagtgtttacggactcggactaccagctcccgtacgtcctcggctctgcgcacgagggctgcctccctccgttcccggcggacgtcttcatgattccgcagtacggctacctgacgctcaacgatggcagccaggccgtgggacgctcatccttttactgcctggaatacttcccttcgcagatgctgagaacgggcaacaactttcagttcagctacgagtttgagaacgtgcctttccacagcagctacgctcacagccagagcctggaccggctgatgaatcctctcatcgaccagtacctgtactacctgagcaagactattaatggatctggacagaatcaacagaccttgaaatttagcgtggccggacctagtaacatggccgttcagggaagaaactacatacctggaccctcataccggcagcagcgcgtttcaacgacagtgacacaaaacaacaacagcgaatttgcctggcctggtgcttcatcttgggctctcaatggccgtaattctctgatgaatcctggcccggctatggcctcacacaaagaaggagaagaccgtttctttcccctgagcggttctctcatttttggcaaacaaggcacaggaagagacaacgtggatgcggacaaggtcatgattaccaacgaagaggaaattaaaaccactaaccctgtggccacggagtcctatgggcaggtggcaaccaaccaccagggccagagtgcgcggggcgacatgctcgggaaggcggcccaaaccggatgggtccaaaaccagggaatcttacctggcatggtgtggcaagacagagacgtgtacctgcagggtcccatttgggccaagattcctcacacggatggaaactttcacccgtctccgctgatgggcggctttggaatgaaacacccgcctcctcagatcctcatcaaaaacacgcctgtacctgcggatcctccgaccgccttcaacaaggacaagctgaactcattcatcacccagtactccactggacaggtcagcgtggagattgagtgggagctgcagaaggaaaacagcaagcgctggaatcccgagatccagtacacatccaactactacaaatctaacaatgtggaatttgctgtgaatactgaaggagtgtatagtgagcctcgccccattggcacccgttacctcacccgtaatctgtaa

SEQ ID NO: 26 - DNA, 2208 bp, Synthetic construct
  source 1..2208: mol_type = other DNA; note = Shuffle32 capsid nucleotide sequence
atggctgccgatggttatcttccagattggctcgaggacaacctttctgaaggcattcgcgagtggtgggacctgaaacctggagcccccaagcccaaggccaaccagcagaagcaggacgacggccggggtctggtgcttcctggctacaagtacctcggacccttcaacggactcgacaagggagagccggtcaacgaggcagacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaaaaagaggcttcttgaacctcttggtctggttgaggaagcggctaagacggctcctgcaaagaagagaccggtagagccgtcacctcagcgttcccccgactcctccacgggcatcggcaagaaaggccagcagcccgccagaaagagactcaatttcggtcagactggcgactcagagtcagttccagacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcaccagaacctgggccctgcccacttacaacaaccacctctacaaacaaatttccagccaatcaggagcctcgaacgacaatcactactttggctacagcaccccttgggggtattttgacttcaacagattccactgccacttttcaccacgtgactggcagcgactcatcaacaacaactggggattccgacccaagagactcaacttcaagctcttcaacatccaagtcaaggaggtcacgacaaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaacggtagtcaggccgtgggacgctcctccttctactgcctggaatactttccttcgcagatgctgagaacgggcaataactttaccttcagctacaccttcgaggacgtgcctttccacagcagctacgcgcacagccagagcctggaccggctgatgaaccctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcgagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggaaatcgagtgggagctgcagaaggagaacagcaaacgctggaacccagagattcagtacacttcaaactactacaaatctacaaatgtggactttgccgttgacagccagggtgtttactctgagcctcgccctattggcactcgttacctcacccgtaatctg

SEQ ID NO: 27 - DNA, 2208 bp, Synthetic construct
  source 1..2208: mol_type = other DNA; note = Shuffle35 capsid nucleotide sequence
atggctgccgatggttatcttccagattggctcgaggacaacctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggcctgtagagcagtctcctcaggaaccggactcctccgcgggtattggcaaatcgggtgcacagcccgctaaaaagagactcaatttcggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcaccagaacctgggccctgcccacttacaacaaccatctctacaagcaaatctccagtgcttcaacgggggccagcaacgacaaccactacttcggctacagcaccccctgggggtattttgactttaacagattccactgccatttctcaccacgtgactggcagcgactcatcaacaacaatgggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccgtcgcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggatggacactttcatccttctcctctgatgggaggctttggactgaaacatccgcctcctcaaatcatgatcaaaaatactccggtaccggcaaatccttcgaccaccttcagtgcggcaaagtttgcttcatttatcactcagtactccactggacaggtcagcgtggaaattgagtgggagctacagaaagaaaacagcaaacgttggaatccagagattcagtacacttccaactactacaaatctacaagtgtggactttgctgttaatacagaaggcgtgtactctgaaccccgccccattggcaccagatacctgactcgtaatctg

SEQ ID NO: 28 - DNA, 30 bp, Synthetic construct
  source 1..30: mol_type = other DNA; note = Pep2hSC1-F
gattcgtggtctgaggcggccgcgaccgca

SEQ ID NO: 29 - DNA, 30 bp, Synthetic construct
  source 1..30: mol_type = other DNA; note = Pep2hSC1-R
accccgcttcctctggccctggaggttggt

SEQ ID NO: 30 - DNA, 30 bp, Synthetic construct
  source 1..30: mol_type = other DNA; note = Pep2hSC2-F
gttgattttgataaggcggccgcgaccgca

SEQ ID NO: 31 - DNA, 30 bp, Synthetic construct
  source 1..30: mol_type = other DNA; note = Pep2hSC2-R
atccttcctcctctggccctggaggttggt

SEQ ID NO: 32 - DNA, 30 bp, Synthetic construct
  source 1..30: mol_type = other DNA; note = Pep9hSC1-F
gacgtgcgcgtccaggcggcccaaaccgga

SEQ ID NO: 33 - DNA, 30 bp, Synthetic construct
  source 1..30: mol_type = other DNA; note = Pep9hSC1-R
gcccctcgcactctggccctggtggttggt

SEQ ID NO: 34 - DNA, 30 bp, Synthetic construct
  source 1..30: mol_type = other DNA; note = Pep9hSC2-F
gatgtggctggggaggcggcccaaaccgga

SEQ ID NO: 35 - DNA, 30 bp, Synthetic construct
  source 1..30: mol_type = other DNA; note = Pep9hSC2-R
accccgacccctctggccctggtggttggt

SEQ ID NO: 36 - DNA, 30 bp, Synthetic construct
  source 1..30: mol_type = other DNA; note = Pep9hSC5-F
cgtcctcctcctcaggcggcccaaaccgga

SEQ ID NO: 37 - DNA, 30 bp, Synthetic construct
  source 1..30: mol_type = other DNA; note = Pep9hSC5-R
atcagcacgactctggccctggtggttggt

SEQ ID NO: 38 - DNA, 30 bp, Synthetic construct
  source 1..30: mol_type = other DNA; note = Pep9hSC9-F
gacatgctcgggaaggcggcccaaaccgga

SEQ ID NO: 39 - DNA, 30 bp, Synthetic construct
  source 1..30: mol_type = other DNA; note = Pep9hSC9-R
gccccgcgcactctggccctggtggttggt

SEQ ID NO: 40 - protein, 14 aa, Synthetic construct
  source 1..14: note = Modified AAV2
QGQRGQAMMAQAAA

(19) United States
(12) Patent Application Publication
(10) Pub. No.: US 2026/0263634 A1
(43) Pub. Date: Sep. 10, 2026

(54) AAV CAPSIDS AND VECTORS FOR TRANSDUCTION OF CELLS OF THE PERIPHERAL NERVOUS SYSTEM

(71) Applicant: CHILDREN'S MEDICAL RESEARCH INSTITUTE, Westmead (AU)

(72) Inventors: Leszek Lisowski, Westmead (AU); Matthieu Drouyer, Westmead (AU); Samantha Ginn, Westmead (AU)

(73) Assignee: CHILDREN'S MEDICAL RESEARCH INSTITUTE, Westmead (AU)

(21) Appl. No.: 19/471,749

(22) PCT Filed: Apr. 3, 2024
(86) PCT No.: PCT/AU2024/050314

§ 371 Date: Oct. 2, 2025

(30) Foreign Application Priority Data
Apr. 4, 2023 (AU) 2023900979

(51) Int. Cl. A61K 48/00; C07K 14/005; C12N 15/86

(52) U.S. Cl. CPC A61K 48/0041; C07K 14/005; C12N 15/86; C12N 2750/14122; C12N 2750/14143; C12N 2750/14145; C12N 2750/14151

(57) ABSTRACT

The present invention relates to novel adeno-associated virus (AAV) variants capable of targeting cells of the peripheral nervous system. Provided herein are AAV capsid polypeptides and encoding nucleic acid molecules, the capsid polypeptides. Also provided are AAV vectors comprising the capsid polypeptides, and nucleic acid vectors (e.g. plasmids) comprising the encoding nucleic acids molecules, as well as host cells comprising the vectors. Also provided are methods and uses of the polypeptides, encoding nucleic acids molecules, vectors and host cells.

### FIELD OF THE DISCLOSURE

The present disclosure relates generally to adeno-associated virus (AAV) capsid polypeptides and encoding nucleic acid molecules. The disclosure also relates to AAV vectors comprising the capsid polypeptides, and nucleic acid vectors (e.g. plasmids) comprising the encoding nucleic acids molecules, as well as to host cells comprising the vectors. The disclosure also relates to methods and uses of the polypeptides, encoding nucleic acids molecules, vectors and host cells.

### BACKGROUND OF THE DISCLOSURE

Gene therapy has most commonly been investigated and achieved using viral vectors, with notable recent advances being based on adeno-associated viral vectors. Adeno-associated virus (AAV) is a replication-deficient parvovirus, the single-stranded DNA genome of which is about 4.7 kb in length. The AAV genome includes inverted terminal repeat (ITRs) at both ends of the molecule, flanking two open reading frames: rep and cap. The cap gene encodes three capsid proteins: VP1, VP2 and VP3. The three capsid proteins typically assemble in a ratio of 1:1:8-10 to form the AAV capsid, although AAV capsids containing only VP3, or VP1 and VP3, or VP2 and VP3, have been produced. The cap gene also encodes the assembly activating protein (AAP) from an alternative open reading frame. AAP promotes capsid assembly, acting to target the capsid proteins to the nucleolus and promote capsid formation. The rep gene encodes four regulatory proteins: Rep78, Rep68, Rep52 and Rep40. These Rep proteins are involved in AAV genome replication.

The ITRs are involved in several functions, in particular integration of the AAV DNA into the host cell genome, as well as genome replication and packaging. When AAV infects a host cell, the viral genome can integrate into the host's chromosomal DNA resulting in latent infection of the cell. Thus, AAV can be exploited to introduce heterologous sequences into cells. In nature, a helper virus (for example, adenovirus or herpesvirus) provides protein factors that allow for replication of AAV virus in the infected cell and packaging of new virions. In the case of adenovirus, genes E1A, E1B, E2A, E4 and VA provide helper functions. Upon infection with a helper virus, the AAV provirus is rescued and amplified, and both AAV and the helper virus are produced.

AAV vectors (also referred to as recombinant AAV, rAAV) that contain a genome that lacks some, most or all of the native AAV genome and instead contains one or more heterologous sequences flanked by the ITRs have been successfully used in gene therapy settings. These AAV vectors are widely used to deliver heterologous nucleic acid to cells of a subject for therapeutic purposes. Recombinant AAV vectors are increasingly showing huge therapeutic promise in targeting organs and cells that necessitate direct in vivo gene delivery, such as the liver and central nervous system cells. An ongoing challenge is enhance vector-mediated gene delivery to target tissues of high therapeutic value that are more difficult-to-access, such cells of the peripheral nervous system.

Schwann cells are the primary glial cell in the peripheral nervous system, secreting key signalling molecules critical for both axonal maintenance and repair. Due to their ability to promote axonal regeneration and remyelination following nerve injury, Schwann cells are the primary clinical target in peripheral nerve-related neuropathies. For example, Charcot-Marie-Tooth (CMT) disease represents a group of inherited demyelinating neuropathies caused by mutations in the PMP22, GJB1 or SH3TC2 genes expressed in myelinating Schwann cells. Another potential target is the tumour forming Schwann cells that underlie neurofibromatosis 1 (NF1), neurofibromatosis 2 (NF2) and schwannomatosis. Although the resulting tumours are typically benign, these debilitating diseases are often disfiguring, cause intractable pain and secondary neurological dysfunction, which significantly impairs patient quality of life. Recent advances in gene therapy technologies have enabled delivery of a functional copy of the disease-causing genes in gene replacement strategies, or alternatively correction, replacement or silencing of disease-causing genes at the endogenous locus. However there remains a need for improved targeting of gene therapy vectors to Schwann cells to more specifically and efficiently deliver therapeutic cargo.

### SUMMARY OF THE DISCLOSURE

The present disclosure is predicated in part on the identification of novel AAV capsid polypeptides. Typically, the capsid polypeptides, when present in the capsid of an AAV vector, facilitate transduction of cells, such as transduction of cells in the peripheral nervous system, in particular Schwann cells. The transduction of Schwann cells by AAV vectors having a capsid comprising a capsid polypeptide of the present disclosure is generally increased or enhanced compared to AAV vectors comprising a reference AAV capsid polypeptide (e.g. the prototypic AAV2 capsid set forth in SEQ ID NO:1 or the AAV9 capsid set forth in SEQ ID NO:2). The capsid polypeptides of the present disclosure are therefore particularly useful in preparing AAV vectors, and in particular, AAV vectors for therapeutic applications. Similarly, AAV vectors comprising a capsid polypeptide of the present disclosure (i.e. having a capsid comprising or consisting of a capsid polypeptide of the present disclosure) are of particular use in gene therapy applications, such as for delivery of heterologous nucleic acids to the peripheral nervous system for the treatment of various diseases and conditions.

In a first aspect, the disclosure provides an AAV capsid polypeptide capable of increasing the transduction efficiency of a cell of the peripheral nervous system of an AAV vector comprising the capsid polypeptide compared to an AAV vector comprising the AAV2 capsid of SEQ ID NO:1 or the AAV9 capsid of SEQ ID NO:2, wherein the capsid polypeptide comprises:
(i) a 12 amino acid substitution at positions 585-589 of SEQ ID NO:1 with a sequence comprising or consisting of the amino acid sequence set forth in SEQ ID NO:3; or
(ii) a 12 amino acid substitution at positions 586-590 of SEQ ID NO:2 with a sequence comprising or consisting of the amino acid sequence set forth in SEQ ID NO:8 or 9.

In an embodiment, the cell of the peripheral nervous system is a Schwann cell.

In an embodiment, the AAV capsid polypeptide comprises, relative to the AAV2 capsid polypeptide set forth in SEQ ID NO:1, a 12 amino acid substitution at positions 585-589 of SEQ ID NO:1 with a sequence comprising or consisting of the amino acid sequence set forth in SEQ ID NO:3, and wherein the capsid polypeptide comprises at least about 80% sequence identity to positions 1-584 and/or 590-735 of SEQ ID NO:1, flanking the amino acid substitution. Optionally the amino acid substitution comprises or consists of the amino acid sequence set forth in SEQ ID NO:4 or 5, or a sequence having at least about 85% sequence identity thereto.

The AAV capsid polypeptide may comprise or consist of the amino acid sequence of SEQ ID NO:1 from positions 1-584 and 590-735 of SEQ ID NO:1, flanking the amino acid substitution. The AAV capsid polypeptide may comprise the amino acid sequence set forth in SEQ ID NO:6 or 7, or a sequence having at least about 80% sequence identity thereto.

In another embodiment of the first aspect, the AAV capsid polypeptide comprises, relative to the AAV9 capsid polypeptide set forth in SEQ ID NO:2, a 12 amino acid substitution at positions 586-590 of SEQ ID NO:2 with a sequence comprising or consisting of the amino acid sequence set forth in SEQ ID NO:8 or 9, and wherein the capsid polypeptide comprises at least about 80% sequence identity to positions 1-585 and/or 591-736 of SEQ ID NO:2, flanking the amino acid substitution. Optionally the amino acid substitution comprises or consists of the amino acid sequence set forth in any one of SEQ ID NOs:10-13, or a sequence having at least about 85% sequence identity thereto.

The AAV capsid polypeptide may comprise or consist of the amino acid sequence of SEQ ID NO:2 from positions 1-585 and 591-736 of SEQ ID NO:2, flanking the amino acid substitution. The AAV capsid polypeptide may comprise the amino acid sequence set forth in any one of SEQ ID NOs:14-17, or a sequence having at least about 80% sequence identity thereto.

A second aspect of the disclosure provides an AAV capsid polypeptide, comprising a peptide modification relative to the AAV2 capsid polypeptide set forth in SEQ ID NO:1, wherein the peptide modification comprises a substitution at amino acid positions 585-589 of SEQ ID NO:1 with a sequence comprising or consisting of the amino acid sequence set forth in SEQ ID NO:3, and wherein the portion of the capsid polypeptide that is not the peptide modification comprises at least about 80% sequence identity to positions 1-584 and/or 590-735 of SEQ ID NO:1.

In an embodiment, the peptide modification comprises or consists of the amino acid sequence set forth in SEQ ID NO:4 or 5, or a sequence having at least about 85% sequence identity thereto.

In an embodiment, the portion of the capsid polypeptide that is not the peptide modification comprises or consists of the amino acid sequence of SEQ ID NO:1 from positions 1-584 and 590-735 of SEQ ID NO:1, flanking the peptide modification. Optionally, the AAV capsid polypeptide comprises the amino acid sequence set forth in SEQ ID NO:6 or 7, or a sequence having at least about 80% sequence identity thereto.

In an embodiment, the capsid polypeptide is capable of increasing the transduction efficiency of a vector comprising the capsid polypeptide compared to an AAV vector comprising a capsid polypeptide comprising the amino acid sequence of SEQ ID NO:1.

A third aspect of the disclosure provides an AAV capsid polypeptide, comprising a peptide modification relative to the AAV9 capsid polypeptide set forth in SEQ ID NO:2, wherein the peptide modification comprises a substitution at amino acid positions 586-590 of SEQ ID NO:2 with a sequence comprising or consisting of the amino acid sequence set forth in SEQ ID NO:8 or 9, and wherein the portion of the capsid polypeptide that is not the peptide modification comprises at least about 80% sequence identity to positions 1-585 and/or 591-736 of SEQ ID NO:2.

In an embodiment, the peptide modification comprises or consists of the amino acid sequence set forth in any one of SEQ ID NOs:10-13, or a sequence having at least about 85% sequence identity thereto.

In an embodiment, the portion of the capsid polypeptide that is not the peptide modification comprises or consists of the amino acid sequence of SEQ ID NO:2 from positions 1-585 and 591-736 of SEQ ID NO:2, flanking the peptide modification. Optionally, the AAV capsid polypeptide may comprise the amino acid sequence set forth in any one of SEQ ID NOs:14-17, or a sequence having at least about 80% sequence identity thereto.

In an embodiment, the capsid polypeptide is capable of increasing the transduction efficiency of a vector comprising the capsid polypeptide compared to an AAV vector comprising a capsid polypeptide comprising the amino acid sequence of SEQ ID NO:2.

A fourth aspect of the disclosure provides an AAV capsid polypeptide comprising an amino sequence as set forth in SEQ ID NO:18 or 19, or a sequence having at least about 80% sequence identity thereto.

A fifth aspect of the disclosure provides an AAV vector, comprising a capsid polypeptide of the disclosure.

In an embodiment, the vector exhibits increased transduction efficiency of a cell of the peripheral nervous system compared to an AAV vector comprising a capsid polypeptide comprising the amino acid sequence of SEQ ID NO:1 or 2. Typically, the cell of the peripheral nervous system is a Schwann cell.

Optionally, transduction efficiency is increased by at least or about 10%, 20%, 30%, 40%, 50%, 60%, 70%, 80%, 90%, 100%, 200%, 300%, 400% or 500%.

In an embodiment, the AAV vector further comprises a heterologous coding sequence. The heterologous coding sequence may encode a peptide, polypeptide or polynucleotide, optionally a therapeutic peptide, polypeptide or polynucleotide.

A sixth aspect of the disclosure provides an isolated nucleic acid molecule encoding a capsid polypeptide of the disclosure.

A seventh aspect provides a vector comprising the nucleic acid molecule of the sixth aspect. In an embodiment, the vector is selected from among a plasmid, cosmid, phage and transposon.

An eighth aspect provides a host cell, comprising the AAV vector of the fifth aspect, the nucleic acid molecule of the sixth aspect, or the vector of the seventh aspect.

A ninth aspect provides a method for introducing a heterologous coding sequence into a host cell, comprising contacting a host cell with the AAV vector of the fifth aspect.

In an embodiment, the host cell is a cell of the peripheral nervous system. The cell may be a Schwann cell, optionally a tumour-forming Schwann cell.

In an embodiment, contacting the host cell with the AAV vector comprises administering the AAV vector to a subject. Administration of the AAV vector to the subject may effect treatment of a disease or condition of the peripheral nervous system.

Also provided is the use of the AAV vector of the fifth aspect for the preparation of a medicament for treating a disease or condition of the peripheral nervous system.

Also provided is a method for producing an AAV vector, comprising culturing a host cell comprising a nucleic acid molecule encoding the capsid polypeptide of the disclosure, an AAV rep gene, a heterologous coding sequence flanked by AAV inverted terminal repeats, and helper functions for generating a productive AAV infection, under conditions suitable to facilitate assembly of an AAV vector comprising a capsid comprising the capsid polypeptide of the disclosure, wherein the capsid encapsidates the heterologous coding sequence. The host cell may be a cell of the peripheral nervous system, optionally a Schwann cell.

### BRIEF DESCRIPTION OF THE DRAWINGS

Embodiments of the disclosure are described herein, by way of non-limiting example only, with reference to the following drawings.

FIG. 1—Functional transduction selection platform for AAV capsids targeting primary human Schwann cells. (A) Overview of the capsid variant selection using the functional transduction (FT) platform. (B) Phylogenic relation of the selected AAV shuffling capsid variants and the parental AAV serotypes used to construct the library. Scale: evolutionary distance of the number of substitutions per site. (C) Schematic representation of the barcoded-AAV ‘hSC Testing Kit’ for NGS comparison in primary human Schwann cells (SCs) isolated from the skin (hSk-SCs) or from the sciatic nerve (hN-SCs) and primary human fibroblasts from different donors. (D) Identity of the amino acid peptide sequences of Pep2hSC1 and Pep2hSC2 inserted in a modified AAV2 capsid. (E and F) Analysis of barcoded variants with capsid recovery achieved at the level of both (E) cell entry (DNA) and (F) transgene expression (mRNA). Heat-map and clustering analysis of capsid performance as a percentage of total NGS reads for each cell type. Values are normalised to ‘pre-mix pool’ and are the average of 2 barcodes.

FIG. 2—AAV capsid alignments. (A) Alignment of amino acid residues between AAV2 (Lco2), Pep2hSC1 and Pep2hSC2 corresponding to positions 557-619 of the AAV2 capsid sequence set forth in SEQ ID NO:1. (B) Alignment of amino acid residues between AAV9 (Lco9), Pep9hSC1, Pep9hSC2, Pep9hSC5 and Pep9hSC9 corresponding to positions 559-620 of the AAV9 capsid sequence set forth in SEQ ID NO:2.

FIG. 3—Heparin completion and neutralization assays. (A) Comparison of recombinant AAV crude production. Data are presented as mean±SEM (n=5 independent single dish lysate). (B) AAV capsid variants Pep2hSC1 and Pep2hSC2 and parental AAV2 were pre-incubated with heparin (500 μg/mL). Transduction efficiency on HEK293T cells was quantified by FACS according to eGFP positive cells 72 h after transduction (n=3 independent experiments). (C) Neutralization assay of indicated AAV capsids following pre-incubation with human IVIg prior to transduction of HEK293T cells. The percentage of GFP-positive cells 72 h after transduction was analyzed by flow cytometry (n=4 independent experiments). The dotted line represents IVIg-mediated inhibition of AAV transduction by 50%. P values were determined by ordinary two-way ANOVA using Sidak's multiple comparison test; ****p≤0.0001. Data is shown as mean±SEM.

FIG. 4—Pep2hSC1 or Pep2hSC2 AAV variants transduce hSCs with high efficiency and specificity. (A) Representative images of pure cultured hSCs transduced with EGFP reporter AAVs packaged using indicated capsids (MOT=1000 vg/cell). Blue: DAPI, purple: S100 (SCs marker), green: AAV-encoded GFP. Arrows show eGFP+/S100− cells. Scale bar: 50 μm. (B) Percentage of eGFP+ SCs. Quantification was performed using 80 cells per image, and ≥3 images per variant. P values were determined by one-way ANOVA with Holm-Śidák's multiple comparison test (**p≤0.01; ***p≤0.001; ****p≤0.0001). Data are given as mean±SEM. (C) Representative immunofluorescence images of mixed cultured hSCs transduced with EGFP reporter AAVs packaged using indicated capsids (MOT=1000 vg/cell). For (A) and (C): arrows indicate eGFP+/S100− cells. Scale bar, 50 μm. (D and E) Percentage of (D) eGFP+/S100+ cells, (E) eGFP+/S100− cells. Quantification was performed using 30 cells per image, and ≥4 images per variant. P values were determined by one-way ANOVA with Holm-Śidák's multiple comparison test (*p≤0.05; **p≤0.01). Data are given as mean±SEM. (F) Proportion of GFP+ cells in the mixed hSC culture. Percentages of S100+ and S100− cells among total eGFP+ cells were calculated. P values were determined by unpaired t-test (*p≤0.5; **p≤0.01; ****p≤0.0001). For (B), (D), and (F), data is represented as mean±SEM.

FIG. 5—Pep2hSC1 and Pep2hSC2 AAV variants showed enhanced transduction in hSCs isolated from NF1 plexiform neurofibroma compared to AAV-DJ and AAV2.7m8. Functional analysis of indicated AAVs in hSC isolated from NF1 plexiform neurofibroma transduced at (A-D) MOT=1,000 or (E-H) MOT=10,000 vg/cell. Representative images of hSCs transduced with indicated AAVs encoding eGFP reporter at (A) MOT=1,000 vg/cell or (E) MOT=10,000 vg/cell. Blue: DAPI, purple: S100 (SCs marker), green: AAV-encoded eGFP. Arrows show eGFP+/S100− cells. Scale bar: 50 μm. (B and C) Percentage of (B) eGFP+/S100+ cells, (C) eGFP+/S100− cells. (D) Proportion of eGFP+ cells in the mixed hSC culture. Percentages of S100+ and S100− cells among total eGFP+ cells were calculated. P values were determined by unpaired t-test (**p≤0.01; ***p≤0.001; ****p≤0.0001). (F and G) Percentage of (F) eGFP+/S100+ cells, (G) eGFP+/S100− cells. (H) Proportion of eGFP+ cells in the mixed hSC culture. Percentages of S100+ and S100− cells among total eGFP+ cells were calculated. P values were determined by unpaired t-test (**p≤0.01; ***p≤0.001; ****p≤0.0001). (B, C, F and G) Quantification was performed using ≥30 cells per image, and 4 images per variant. P values were determined by one-way ANOVA with Holm-Śidák's's multiple comparison test (*p≤0.05; **p≤0.01). Data shown as mean±SEM.

FIG. 6—Pep2hSC1 or Pep2hSC2 AAV variants transduce Schwann cells in human nerve segments. (A) Number of eGFP+ cells overlapping with DAPI marker in immunofluorescently labelled nerve segments 14 days post injection from three donors (mean±SEM; n=3). Nerve segments (0.5 cm) were injected with AAV-DJ, Pep2hSC1 or Pep2hSC2 vectors encoding a CMV-GFP transgene (1×10¹⁰ vg dose per segment). (B) Confocal microscopy images of longitudinal sections immunostained for GFP (green) with either S100 (purple) for SCs, PRX (yellow) for myelinating SCs, or P75 (red) for non-myelinating SCs. Blue outlined insets show magnified area of colocalization. Scale bar: 50 μm. Donor #15: 42-year-old male; Donor #16: 44-year-old male, Caucasian; Donor #17: 73-year-old male, Asian. (C) Pearson correlation coefficient analysis of immunostained sections (from B) to determine the type of hSCs (PRX- or P75-positive) that were transduced.

FIG. 7—Evaluation of Pep2hSC1 or Pep2hSC2 AAV variants in sciatic nerve crush model. (A) Illustration of AAV delivery following nerve crush injury. Following forceps-induced nerve injury, AAV-DJ, Pep2hSC1 or Pep2hSC2 were administered by intraneural injection (2×10¹⁰ vg dose per mouse) and sciatic nerves were harvested 4 weeks post injection. (B) Quantification of the percentage of transduced area in proximal and distal region from crushed site (transduction area has been reported as the percentage eGFP stained area versus total nerve area). (C) Quantification of the mean eGFP intensity per transduced cells. (D and E) Longitudinal sections were stained with DAPI (blue), eGFP (green), PRX (magenta) and neurofilament (NF) staining (light blue). Representative images of (D) proximal region and (E) distal region from crushed site. Insets show a magnified views of selected areas highlighted by white dotted outline. Arrowheads indicate colocalization between eGFP and PRX (myelinating SC marker). Arrows show eGFP-labeled cells enclose the axons marked by neurofilament (NF) staining. Scale bar: 50 μm.

FIG. 8—Evaluation of Pep2hSC1 or Pep2hSC2 AAV variants in the humanized FRG (hFRG) model. AAV vector genomes per diploid cell in human hepatocyte cells. Quantification of the percentage of transduced human hepatocytes. Individual data points represent the average of 11-15 human clusters per mouse. Data are represented as mean±SEM.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e. at least one or more than one) unless the context clearly dictates otherwise. Thus, for example, reference to “a polypeptide” includes a single polypeptide, as well as two or more polypeptides.

In the context of this specification, the term “about,” is understood to refer to a range of numbers that a person of skill in the art would consider equivalent to the recited value in the context of achieving the same function or result.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, a “vector” includes reference to both polynucleotide vectors and viral vectors, each of which are capable of delivering a transgene contained within the vector into a host cell. Vectors can be episomal, i.e., do not integrate into the genome of a host cell, or can integrate into the host cell genome. The vectors may also be replication competent or replication deficient. Exemplary polynucleotide vectors include, but are not limited to, plasmids, cosmids and transposons. Exemplary viral vectors include, for example, AAV, lentiviral, retroviral, adenoviral, herpes viral and hepatitis viral vectors.

As used herein, “adeno-associated viral vector” or “AAV vector” refers to a vector in which the capsid is derived from an adeno-associated virus, including without limitation, AAV1, AAV2, AAV3, AAV4, AAV5, AAV6, AAV7, AAV8, AAV9, AAV10, AAV11, AAV12 or AAV13, AAV from other clades or isolates, or is derived from synthetic, bioengineered or modified AAV capsid proteins, including chimeric capsid proteins. In particular embodiments, the AAV vector has a capsid comprising a capsid polypeptide of the present disclosure. When referring to AAV vectors, both the source of the genome and the source of the capsid can be identified, where the source of the genome is the first number designated and the source of the capsid is the second number designated. Thus, for example, a vector in which both the capsid and genome are derived from AAV2 is more accurately referred to as AAV2/2. A vector with an AAV6-derived capsid and an AAV2-derived genome is most accurately referred to as AAV2/6. A vector with the bioengineered DJ capsid and an AAV2-derived genome is most accurately referred to as AAV2/DJ. For simplicity, and because most vectors use an AAV2-derived genome, it is understood that reference to an AAV6 vector generally refers to an AAV2/6 vector, reference to an AAV2 vector generally refers to an AAV2/2 vector, etc. An AAV vector may also be referred to herein as “recombinant AAV”, “rAAV”, “recombinant AAV virion”, “rAAV virion”, “AAV variant”, “recombinant AAV variant”, and “rAAV variant” terms which are used interchangeably and refer to a replication-defective virus that includes an AAV capsid shell encapsidating an AAV genome. The AAV vector genome (also referred to as vector genome, recombinant AAV genome or rAAV genome) comprises a transgene flanked on both sides by functional AAV ITRs. Typically, one or more of the wild-type AAV genes have been deleted from the genome in whole or part, preferably the rep and/or cap genes. Functional ITR sequences are necessary for the rescue, replication and packaging of the vector genome into the rAAV virion.

A “conservative amino acid substitution” is one in which the amino acid residue is replaced with an amino acid residue having a similar side chain. Families of amino acid residues having similar side chains have been defined in the art. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity.

The term “ITR” refers to an inverted terminal repeat at either end of the AAV genome. This sequence can form hairpin structures and is involved in AAV DNA replication and rescue, or excision, from prokaryotic plasmids. ITRs for use in the present disclosure need not be the wild-type nucleotide sequences, and may be altered, e.g., by the insertion, deletion or substitution of nucleotides, as long as the sequences provide for functional rescue, replication and packaging of rAAV.

As used herein, “functional” with reference to a capsid polypeptide means that the polypeptide can self-assemble or assemble with different capsid polypeptides to produce the proteinaceous shell (capsid) of an AAV virion. It is to be understood that not all capsid polypeptides in a given host cell assemble into AAV capsids. Preferably, at least 25%, at least 50%, at least 75%, at least 85%, at least 90%, at least 95% of all AAV capsid polypeptide molecules assemble into AAV capsids. Suitable assays for measuring this biological activity are described e.g. in Smith-Arica and Bartlett (2001), Curr Cardiol Rep 3(1): 43-49.

“AAV helper functions” or “helper functions” refer to functions that allow AAV to be replicated and packaged by a host cell. AAV helper functions can be provided in any of a number of forms, including, but not limited to, as a helper virus or as helper virus genes which aid in AAV replication and packaging. Helper virus genes include, but are not limited to, adenoviral helper genes such as E1A, E1B, E2A, E4 and VA. Helper viruses include, but are not limited to, adenoviruses, herpesviruses, poxviruses such as vaccinia, and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Baculoviruses available from depositories include Autographa californica nuclear polyhedrosis virus.

As used herein, the term “transduction” refers to the ability of an AAV vector to enter one or more particular cell types and transfer the DNA contained within the AAV vector into the cell. Transduction can be assessed by measuring the amount of AAV DNA or RNA expressed from the AAV DNA in a cell or population of cells, and/or by assessing the number of cells in a population that contain AAV DNA or RNA expressed from the DNA. Where the presence or amount of RNA is assessed, the type of transduction assessed is referred to herein as “functional transduction”, i.e. the ability of the AAV to transfer DNA to the cell and have that DNA expressed. “Transduction efficiency” is a measure of the level of transduction from a starting amount of AAV vector (e.g. the starting amount of vector being injected in vivo or applied to cells in vitro), and can be quantitative or qualitative, and/or with reference to a particular control, e.g. a prototypic AAV vector. For example, if a candidate AAV vector transduces twice as many cells as a control vector and/or the amount of AAV DNA per cell from transduction with the candidate AAV vector is twice that of transduction with the control vector, where the starting amount of each vector was the same (i.e. the amount of each vector injected into a subject or applied to cells was the same), it can be said that the transduction efficiency of the candidate AAV vector is 200% greater than, or is twice that of, the transduction efficiency of the control vector.

The phrase “numbering relative to” a sequence, such as SEQ ID NO:1, means that the numbering of the amino acid position being referred to is as shown in the sequence, e.g. SEQ ID NO:1. It will be appreciated that the sequence is simply a reference sequence, and that the same amino acid residue or position may correspond to a different number in a different sequence, such as if the different sequence is a truncated form or is a sequence that has insertions or deletions compared to the reference sequence. To identify corresponding positions or residues in different sequences, sequences of related or variant polypeptides are aligned by any method known to those of skill in the art. Such methods typically maximize matches (e.g. identical nucleotides or amino acids at positions), and include methods such as using manual alignments and by using the numerous alignment programs available (for example, BLASTP, ClustlW, ClustlW2, EMBOSS, LALIGN, Kalign, etc.) and others known to those of skill in the art. By aligning the sequences of polypeptides, one skilled in the art can identify corresponding positions.

As used herein, “corresponding nucleotides” or “corresponding amino acid residues” or grammatical variations thereof refer to nucleotides or amino acids that occur at aligned loci. The sequences of related or variant polynucleotides or polypeptides are aligned by any method known to those of skill in the art. Such methods typically maximize matches (e.g. identical nucleotides or amino acids at positions), and include methods such as using manual alignments and by using the numerous alignment programs available (for example, BLASTN, BLASTP, ClustlW, ClustlW2, EMBOSS, LALIGN, Kalign, etc) and others known to those of skill in the art. By aligning the sequences of polynucleotides or polypeptides, one skilled in the art can identify corresponding nucleotides or amino acids. For example, by aligning the prototypic AAV2 capsid polypeptide set forth in SEQ ID NO:1 with another AAV capsid polypeptide, such as the variant set forth in SEQ ID NO: 6 or 7, one of skill in the art can identify regions or amino acids residues within the other AAV polypeptide that correspond to various regions or residues in the AAV polypeptide set forth in SEQ ID NO:1. In another example, by aligning the prototypic AAV9 capsid polypeptide set forth in SEQ ID NO:2 with another AAV capsid polypeptide, such as the variant set forth in any one of SEQ ID Nos: 14-17, one of skill in the art can identify regions or amino acids residues within the other AAV polypeptide that correspond to various regions or residues in the AAV polypeptide set forth in SEQ ID NO:2.

The term “peptide modification” refers to a modification in a polypeptide that involves two or more contiguous amino acids (i.e. that involves a peptide within the polypeptide). The peptide modification can include amino acid insertions, deletions and/or substitutions relative to a reference polypeptide. For example, an exemplary peptide modification of the present disclosure in the AAV2 capsid comprises the inclusion of 12 consecutive amino acid residues, relative to the AAV2 capsid sequence of SEQ ID NO:1, in which 5 residues (corresponding to positions 585-589 of the AAV2 sequence of SEQ ID NO:1) are amino acid substitutions relative to the AAV2 capsid sequence of SEQ ID NO:1 and the next 7 residues are insertions relative to the AAV2 capsid of SEQ ID NO:1. This 12 amino acid peptide modification is also referred to herein as a substitution, in which the amino acids at positions 585-589 of the AAV2 sequence of SEQ ID NO:1 are replaced (substituted) with a 12 amino acid sequence of SEQ ID NO:3. Also by way of example, an exemplary peptide modification of the present disclosure in the AAV9 capsid comprises the inclusion of 12 consecutive amino acid residues, relative to the AAV9 capsid sequence of SEQ ID NO:2, in which 5 residues (corresponding to positions 586-590 of the AAV9 sequence of SEQ ID NO:2) are amino acid substitutions relative to the AAV9 capsid sequence of SEQ ID NO:2 and the next 7 residues are insertions relative to the AAV9 capsid of SEQ ID NO:2. This 12 amino acid peptide modification is also referred to herein as a substitution, in which the amino acids at positions 586-590 of the AAV9 sequence of SEQ ID NO:2 are replaced (substituted) with a 12 amino acid sequence of SEQ ID NO:8 or 9.

A “heterologous coding sequence” as used herein refers to nucleic acid sequence present in a polynucleotide, vector, or host cell that is not naturally found in the polynucleotide, vector, or host cell or is not naturally found at the position that it is at in the polynucleotide, vector, or host cell, i.e. is non-native. A “heterologous coding sequence” can encode a peptide or polypeptide, or a polynucleotide that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous sequence and the genomic DNA can occur. In one example, the heterologous coding sequence is a functional copy of a gene for introduction into a cell that has a defective/mutated copy.

As used herein, the term “operably-linked” with reference to a promoter and a coding sequence means that the transcription of the coding sequence is under the control of, or driven by, the promoter.

The term “host cell” refers to a cell, such as a mammalian cell, that has introduced into it the exogenous DNA, such as a vector or other polynucleotide. The term includes the progeny of the original cell into which the exogenous DNA has been introduced. Thus, a “host cell” as used herein generally refers to a cell that has been transfected or transduced with exogenous DNA.

As used herein, “isolated” with reference to a polynucleotide or polypeptide means that the polynucleotide or polypeptide is substantially free of cellular material or other contaminating proteins from the cells from which the polynucleotide or polypeptide is derived, or substantially free from chemical precursors or other chemicals when chemically synthesized.

The term “subject” as used herein refers to an animal, in particular a mammal and more particularly a primate including a lower primate and even more particularly, a human who can benefit from the present invention. A subject, regardless of whether a human or non-human animal or embryo, may be referred to as an individual, subject, animal, patient, host or recipient. The present disclosure has both human and veterinary applications. For convenience, an “animal” specifically includes livestock animals such as cattle, horses, sheep, pigs, camelids, goats and donkeys, as well as domestic animals, such as dogs and cats. With respect to horses, these include horses used in the racing industry as well as those used recreationally or in the livestock industry. Examples of laboratory test animals include mice, rats, rabbits, guinea pigs and hamsters. Rabbits and rodent animals, such as rats and mice, provide a convenient test system or animal model as do primates and lower primates. In some embodiments, the subject is human.

It will be appreciated that the above described terms and associated definitions are used for the purpose of explanation only and are not intended to be limiting.

TABLE 1
Description of Sequences

| SEQ ID NO | Sequence description |
|---|---|
| 1 | AAV2 capsid (VP1 protein) |
| 2 | AAV9 capsid (VP1 protein) |
| 3 | AAV2 substitution consensus |
| 4 | Pep2hSC1 substitution |
| 5 | Pep2hSC2 substitution |
| 6 | Pep2hSC1 capsid polypeptide |
| 7 | Pep2hSC2 capsid polypeptide |
| 8 | AAV9 substitution consensus sequence 1 |
| 9 | AAV9 substitution consensus sequence 2 |
| 10 | Pep9hSC1 substitution |
| 11 | Pep9hSC2 substitution |
| 12 | Pep9hSC5 substitution |
| 13 | Pep9hSC9 substitution |
| 14 | Pep9hSC1 capsid polypeptide |
| 15 | Pep9hSC2 capsid polypeptide |
| 16 | Pep9hSC5 capsid polypeptide |
| 17 | Pep9hSC9 capsid polypeptide |
| 18 | Shuffle32 capsid polypeptide |
| 19 | Shuffle35 capsid polypeptide |
| 20 | Pep2hSC1 capsid nucleotide sequence |
| 21 | Pep2hSC2 capsid nucleotide sequence |
| 22 | Pep9hSC1 capsid nucleotide sequence |
| 23 | Pep9hSC2 capsid nucleotide sequence |
| 24 | Pep9hSC5 capsid nucleotide sequence |
| 25 | Pep9hSC9 capsid nucleotide sequence |
| 26 | Shuffle32 capsid nucleotide sequence |
| 27 | Shuffle35 capsid nucleotide sequence |
| 28 | Pep2hSC1-F primer |
| 29 | Pep2hSC1-R primer |
| 30 | Pep2hSC2-F primer |
| 31 | Pep2hSC2-R primer |
| 32 | Pep9hSC1-F primer |
| 33 | Pep9hSC1-R primer |
| 34 | Pep9hSC2-F primer |
| 35 | Pep9hSC2-R primer |
| 36 | Pep9hSC5-F primer |
| 37 | Pep9hSC5-R primer |
| 38 | Pep9hSC9-F primer |
| 39 | Pep9hSC9-R primer |

### Capsid Polypeptides

A central feature of rAAVs is their capsid-driven tissue tropisms (Srivastava, 2016, Curr Opin Virol 21, 75-90). Several AAV vectors have demonstrated tropism for Schwann cells (SCs) with different degree of success between variants. Notably, AAV1, AAV2, AAV6, and AAV-DJ have been shown to transduce primary hSCs with AAV6 and AAV-DJ being the most efficient (Bai et al., 2019, Gene Ther doi:10.1038/s41434-019-0080-9). In rat nerve explants, AAV1, AAV5, AAV6, AAV7, AAV8 and AAV9 were the most efficient variants whereas in human nerve explants, AAV2 is shown to be the most effective (Hoyng et al., 2015, Gene Ther 22, 767-780). AAV9, in combination with the Mpz promoter, has been used in mice to target SCs in X-linked CMT after a single lumbar intrathecal injection (Kagiava et al, 2021, Gene Ther 28, 659-675). AAV9 has also been shown to transduce SCs in mice, rats and non-human primates (NHPs) following intraneural injection. In addition, a single injection of AAV9 in a rat model of CMT1A has been shown to prevent dysfunctions in CMT1A (Gautier et al., 2021, Nat Commun 12, 2356). However, despite promising results in these preclinical models, the aforementioned AAV variants lack specificity and show broad off-target tropism, significantly lowering their translational potential.

Recent advances in AAV capsid bioengineering strategies, including rational design and directed evolution, have demonstrated the ability to select for novel clinically translatable properties of rAAV (Lisowski et al., 2014, Nature 506, 382-386). However, the success of such strategies depends on the interplay of three critical elements: the starting AAV library, the selection strategy or platform and the preclinical model on which the selection and subsequent validation of AAV variants are performed (Westhaus et al., 2022, Hum Gene Ther doi:10.1089/hum.2021.278).

As described herein, the present inventors have developed novel AAV variants capable of targeting primary human Schwann cells (SCs) with high efficiency and specificity for translational applications. Using their proprietary functional transduction (FT)-RNA selection method (see Westhaus et al., 2022, Hum Gene Ther doi:10.1089/hum.2021.278; International Patent Application no. PCT/AU2019/051133, the disclosure of which is incorporated herein in its entirety) to select a highly variable AAV2-based peptide display library in purified primary hSC cultures, and following two rounds of selection, the inventors identified two new AAV2 capsid variants, named Pep2hSC1 and Pep2hSC2, which displayed conserved potency for delivery across a variety of in vitro, in vivo and ex vivo models of SCs. These variants also demonstrated a decrease in targeting human fibroblasts and primary human hepatocytes. Based on their high efficiency and specificity, these two new variants hold a great promise to enable development of the first SC-specific gene therapies in human patients. The amino acid sequences of the Pep2hSC1 and Pep2hSC2 capsids are set forth in SEQ ID Nos:6 and 7, respectively, and the polynucleotide sequences encoding these capsids are set forth in SEQ ID Nos:20 and 21, respectively.

Also identified from an AAV9-based peptide display library using the (FT)-RNA selection method were four new AAV9 capsid variants, named Pep9hSC1, Pep9hSC2, Pep9hSC5 and Pep9hSC9. The amino acid sequences of the Pep9hSC1, Pep9hSC2, Pep9hSC5 and Pep9hSC9 capsids are set forth in SEQ ID Nos:14-17, respectively, and the polynucleotide sequences encoding these capsids are set forth in SEQ ID Nos:22-25, respectively.

Also identified from a DNA shuffled library based on the capsid genes from AAV serotypes 1 to 12, using the (FT)-RNA selection method were two new shuffle capsid variants, named Shuffle32 and Shuffle35. The amino acid sequences of the Shuffle32 and Shuffle35 capsids are set forth in SEQ ID Nos:18 and 19, respectively, and the polynucleotide sequences encoding these capsids are set forth in SEQ ID Nos:26 and 27, respectively.

The present disclosure is predicated in part on the identification of novel AAV capsid polypeptides. Typically, the capsid polypeptides, when present in the capsid of an AAV vector, facilitate transduction of cells of the peripheral nervous system, in particular Schwann cells. Thus, in a particular aspect, the present disclosure relates to AAV capsid polypeptides that facilitate transduction of peripheral nervous system cells, such as Schwann cells. In particular embodiments, the transduction of cells by AAV vectors having a capsid comprising a capsid polypeptide of the present disclosure is generally increased or enhanced compared to AAV vectors comprising a reference AAV capsid polypeptide (e.g. the AAV2 capsid of SEQ ID NO:1 or the AAV9 capsid of SEQ ID NO:2). Transduction or transduction efficiency of AAV vectors can be increased by at least or about 10%, 20%, 30%, 40%, 50%, 60%, 70%, 80%, 90%, 100%, 200%, 300%, 400%, 500%, 600%, 700%, 800%, 900%, 1000% or more. For example, an AAV vector comprising a capsid polypeptide of the present disclosure can be at least or about 1.2×, 1.5×, 2×, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100× or more efficient at transducing cells in vivo compared to an AAV vector comprising a reference AAV capsid polypeptide (e.g. the AAV2 capsid of SEQ ID NO:1 or the AAV9 capsid of SEQ ID NO:2).

The capsid polypeptides of the present disclosure are therefore particularly useful in preparing AAV vectors, such as AAV vectors for therapy of diseases or conditions of the peripheral nervous system, whereby delivery of heterologous nucleic acid into at one cell of the peripheral nervous system, optionally a Schwann cell, by the AAV vector facilitates therapy.

In one aspect, the present disclosure provides an AAV capsid polypeptide, comprising a peptide modification relative to the AAV2 capsid polypeptide set forth in SEQ ID NO:1, wherein the peptide modification comprises a substitution at amino acid positions 585-589 of SEQ ID NO:1 with a sequence comprising or consisting of the amino acid sequence set forth in SEQ ID NO:3, and wherein the portion of the capsid polypeptide that is not the peptide modification comprises at least about 80% sequence identity to positions 1-584 and/or 590-735 of SEQ ID NO:1. The peptide modification comprises 12 consecutive amino acid residues which replace amino acid residues 585-589 of SEQ ID NO: 1, wherein 5 residues are amino acid substitutions of amino acid residues corresponding to positions 585-589 of the AAV2 capsid sequence of SEQ ID NO:1 and the next 7 residues are insertions of amino acids between positions 589 and 590 of the AAV2 capsid sequence of SEQ ID NO:1.

The amino acid sequence of SEQ ID NO:3 is represented by GQRX₁X₂X₃X₄X₅X₆X₇X₈A, where X₁ and X₂ are each K or R, X₃ is G or D, X₄ is D or V, X₅ is S or D, X₆ is W or F, X₇ is S or D and X₈ is E or K. In exemplary capsid polypeptides the peptide modification may comprise 1, 2 or 3 conservative amino acid substitutions of the sequence of SEQ ID NO:3. In particular exemplary capsid polypeptides the peptide modification comprises or consists of the sequence set forth in SEQ ID NO:4 or 5, or a sequence having at least or about 85%, 86%, 87%, 88%, 89%, 90%, 91%, 92%, 93%, 94%, 95%, 96%, 97%, 98% or 99% sequence identity to the sequence of SEQ ID NO:4 or 5.

The backbone of the capsid, i.e. the capsid residues other than those of the peptide modification may comprise amino acid residues 1-584 and/or residues 590-735 of the AAV2 capsid of SEQ ID NO:1, or sequences having at or about 80%, 81%, 82%, 83%, 84%, 85%, 86%, 87%, 88%, 89%, 90%, 91%, 92%, 93%, 94%, 95%, 96%, 97%, 98% or 99% sequence identity to these sequence of SEQ ID NO:1. The backbone of the capsid polypeptide can be from any naturally occurring or modified AAV capsid polypeptide, such as NP40, NP59 or LK03 (see e.g. Paulk et al. 2018, Mol Ther. 26(1):289-303; and Lisowski et al., 2014, Nature 506:382-386), or any modified capsid polypeptide described in International Patent Application No. PCT/AU2021/050158. The capsid polypeptide may comprise the full length VP1 (i.e. corresponding to positions 1-735 of the AAV2 capsid set forth in SEQ ID NO:1), or a fragment thereof, such as the VP2 (i.e. corresponding to positions 138-735 of the AAV2 capsid set forth in SEQ ID NO: 1) or the VP3 (i.e. corresponding to positions 203-735 of the AAV2 capsid set forth in SEQ ID NO:1).

An exemplary capsid polypeptide of the disclosure is capsid Pep2hSC1 comprising the amino acid sequence set forth in SEQ ID NO:6. Pep2hSC1 comprises a peptide modification relative to the AAV2 capsid of SEQ ID NO:1, wherein the peptide modification comprises the sequence of SEQ ID NO:4, which includes amino acid substitutions at positions 585-589 relative to the AAV2 capsid polypeptide of SEQ ID NO:1, and a 7 amino acid insertion after position 589 relative to the AAV2 capsid polypeptide of SEQ ID NO:1. Also encompassed are polypeptides having at least or about 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO:6.

Another exemplary capsid polypeptide of the disclosure is capsid Pep2hSC2 comprising the amino acid sequence set forth in SEQ ID NO:7. Pep2hSC2 comprises a peptide modification relative to the AAV2 capsid of SEQ ID NO:1, wherein the peptide modification comprises the sequence of SEQ ID NO:5, which includes amino acid substitutions at positions 585-589 relative to the AAV2 capsid polypeptide of SEQ ID NO:1, and a 7 amino acid insertion after position 589 relative to the AAV2 capsid polypeptide of SEQ ID NO:1. Also encompassed are polypeptides having at least or about 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO:7.

In another aspect, the present disclosure provides an AAV capsid polypeptide, comprising a peptide modification relative to the AAV9 capsid polypeptide set forth in SEQ ID NO:2, wherein the peptide modification comprises a substitution at amino acid positions 586-590 of SEQ ID NO:2 with a sequence comprising or consisting of the amino acid sequence set forth in SEQ ID NO:8 or 9, and wherein the portion of the capsid polypeptide that is not the peptide modification comprises at least about 80% sequence identity to positions 1-585 and/or 591-736 of SEQ ID NO:2. The peptide modification comprises 12 consecutive amino acid residues which replace amino acid residues 586-590 of SEQ ID NO:2, wherein 5 residues are amino acid substitutions of amino acid residues corresponding to positions 586-590 of the AAV9 capsid sequence of SEQ ID NO:2 and the next 7 residues are insertions of amino acids between positions 590 and 591 of the AAV9 capsid sequence of SEQ ID NO:2.

The amino acid sequence of SEQ ID NO:8 is represented by GQX₁X₂X₃X₄X₅X₆X₇X₈ X₉A, where X₁ is S or R, X₂ is A, G or R, X₃ is A or R, X₄ is G or D, X₅ is D or R, X₆ is V, P or M, X₇ is R, A, P or L, X₈ is V, G or P and X₉ is Q, E or K. The amino acid sequence of SEQ ID NO:9 is represented by GQSX₁RGDX₂X₃X₄X₅A, where X₁ is A or G, X₂ is V or M, X₃ is R, A or L, X₄ is V or G and X₅ is Q, E or K. In exemplary capsid polypeptides the peptide modification may comprise 1, 2 or 3 conservative amino acid substitutions of the sequence of SEQ ID NO:8 or 9. In particular exemplary capsid polypeptides the peptide modification comprises or consists of the sequence set forth in any one of SEQ ID Nos:10-13, or a sequence having at least or about 85%, 86%, 87%, 88%, 89%, 90%, 91%, 92%, 93%, 94%, 95%, 96%, 97%, 98% or 99% sequence identity to the sequence of any one of SEQ ID Nos:10-13.

The backbone of the capsid, i.e. the capsid residues other than those of the peptide modification may comprise may comprise amino acid residues 1-585 and/or residues 591-736 of the AAV9 capsid of SEQ ID NO:2, or sequences having at or about 80%, 81%, 82%, 83%, 84%, 85%, 86%, 87%, 88%, 89%, 90%, 91%, 92%, 93%, 94%, 95%, 96%, 97%, 98% or 99% sequence identity to these sequence of SEQ ID NO:2. The backbone of the capsid polypeptide can be from any naturally occurring or modified AAV capsid polypeptide, such as NP40, NP59 or LK03 (see e.g. Paulk et al. 2018, Mol Ther. 26(1):289-303; and Lisowski et al., 2014, Nature 506:382-386), or any modified capsid polypeptide described in International Patent Application No. PCT/AU2021/050158. The capsid polypeptide may comprise the full length VP1 (i.e. corresponding to positions 1-736 of the AAV9 capsid set forth in SEQ ID NO:2), or a fragment thereof, such as the VP2 (i.e. corresponding to positions 138-736 of the AAV9 capsid set forth in SEQ ID NO:2) or the VP3 (i.e. corresponding to positions 203-736 of the AAV9 capsid set forth in SEQ ID NO:2).

An exemplary capsid polypeptide of the disclosure is capsid Pep9hSC1 comprising the amino acid sequence set forth in SEQ ID NO:14. Pep9hSC1 comprises a peptide modification relative to the AAV9 capsid of SEQ ID NO:2, wherein the peptide modification comprises the sequence of SEQ ID NO:10, which includes amino acid substitutions at positions 586-590 relative to the AAV9 capsid polypeptide of SEQ ID NO:2, and a 7 amino acid insertion after position 590 relative to the AAV9 capsid polypeptide of SEQ ID NO:2. Also encompassed are polypeptides having at least or about 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO:14.

Another exemplary capsid polypeptide of the disclosure is capsid Pep9hSC2 comprising the amino acid sequence set forth in SEQ ID NO:15. Pep9hSC2 comprises a peptide modification relative to the AAV9 capsid of SEQ ID NO:2, wherein the peptide modification comprises the sequence of SEQ ID NO:11, which includes amino acid substitutions at positions 586-590 relative to the AAV9 capsid polypeptide of SEQ ID NO:2, and a 7 amino acid insertion after position 590 relative to the AAV9 capsid polypeptide of SEQ ID NO:2. Also encompassed are polypeptides having at least or about 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO:15.

Another exemplary capsid polypeptide of the disclosure is capsid Pep9hSC5 comprising the amino acid sequence set forth in SEQ ID NO:16. Pep9hSC5 comprises a peptide modification relative to the AAV9 capsid of SEQ ID NO:2, wherein the peptide modification comprises the sequence of SEQ ID NO:12, which includes amino acid substitutions at positions 586-590 relative to the AAV9 capsid polypeptide of SEQ ID NO:2, and a 7 amino acid insertion after position 590 relative to the AAV9 capsid polypeptide of SEQ ID NO:2. Also encompassed are polypeptides having at least or about 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO:16.

Another exemplary capsid polypeptide of the disclosure is capsid Pep9hSC9 comprising the amino acid sequence set forth in SEQ ID NO:17. Pep9hSC9 comprises a peptide modification relative to the AAV9 capsid of SEQ ID NO:2, wherein the peptide modification comprises the sequence of SEQ ID NO:13, which includes amino acid substitutions at positions 586-590 relative to the AAV9 capsid polypeptide of SEQ ID NO:2, and a 7 amino acid insertion after position 590 relative to the AAV9 capsid polypeptide of SEQ ID NO:2. Also encompassed are polypeptides having at least or about 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO:17.

Also provided herein is an AAV capsid polypeptide comprising the amino acid sequence set forth in SEQ ID NO:18, or a having at least or about 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO: 18.

Also provided herein is an AAV capsid polypeptide comprising the amino acid sequence set forth in SEQ ID NO:19, or a having at least or about 80%, 81%, 82%, 83%, 84%, 85%, 86%, 87%, 88%, 89%, 90%, 91%, 92%, 93%, 94%, 95%, 96%, 97%, 98% or 99% sequence identity to the sequence of SEQ ID NO:19.

Also provided are nucleic acid molecules, including isolated nucleic acid molecules, encoding a capsid polypeptide of the disclosure. Thus, amongst the nucleic acid molecules provided herein are those encoding a capsid polypeptide comprising the VP1, VP2 and/or VP3 of any one of the capsid polypeptides set forth in SEQ ID Nos:6,7 and 14-19 as described above or a polypeptides having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity thereto. Non-limiting examples of nucleic acid molecules therefore include those set forth in SEQ ID Nos:20-27, those having at least or about 85%, 86%, 87%, 88%, 89%, 90%, 91%, 92%, 93%, 94%, 95%, 96%, 97%, 98%, or 99% sequence identity thereto, and those that hybridize with medium or high stringency to nucleic acid molecules comprising a sequence set forth in any one of SEQ ID Nos:20-27.

### Vectors

The present disclosure also provides vectors comprising a nucleic acid molecule that encodes a capsid polypeptide described herein, and vectors comprising a capsid polypeptide described herein. The vectors include nucleic acid vectors that comprise a nucleic acid molecule that encodes a capsid polypeptide described herein, and AAV vectors that have a capsid comprising a capsid polypeptide described herein.

### Nucleic Acid Vectors

Vectors of the present disclosure include nucleic acid vectors that comprise a polynucleotide that encodes all or a portion of a capsid polypeptide described herein. The vectors can be episomal vectors (i.e., that do not integrate into the genome of a host cell) or can be vectors that integrate into the host cell genome. Exemplary vectors that comprise a nucleic acid molecule encoding a capsid polypeptide include, but are not limited to, plasmids, cosmids, transposons and artificial chromosomes. In particular examples, the vectors are plasmids.

Vectors, such as plasmids, suitable for use in bacterial, insect and mammalian cells are widely described and well-known in the art. Those skilled in the art would appreciate that vectors of the present disclosure may also contain additional sequences and elements useful for the replication of the vector in prokaryotic and/or eukaryotic cells, selection of the vector and the expression of a heterologous sequence in a variety of host cells. For example, the vectors of the present disclosure can include a prokaryotic replicon (that is, a sequence having the ability to direct autonomous replication and maintenance of the vector extra-chromosomally in a prokaryotic host cell, such as a bacterial host cell). Such replicons are well known in the art. In some embodiments, the vectors can include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein. The nucleic acid vectors will likely also comprise other elements, including any one or more of those described below. Most typically, the vectors will comprise a promoter operably linked to the nucleic acid encoding the capsid protein.

The nucleic acid vectors of the present disclosure can be constructed using known techniques, including, without limitation, the standard techniques of restriction endonuclease digestion, ligation, transformation, plasmid purification, in vitro or chemical synthesis of DNA, and DNA sequencing. The vectors of the present disclosure may be introduced into a host cell using any method known in the art. Accordingly, the present disclosure is also directed to host cells comprising a vector or nucleic acid described herein.

### AAV Vectors

Provided herein are AAV vectors comprising a capsid polypeptide described herein. Methods for vectorizing a capsid protein are well known in the art and any suitable method can be employed for the purposes of the present disclosure. For example, the cap gene can be recovered (e.g. by PCR or digest with enzymes that cut upstream and downstream of cap) and cloned into a packaging construct containing rep. Any AAV rep gene may be used, including, for example, a rep gene is from AAV1, AAV2, AAV3, AAV4, AAV5, AAV6, AAV7, AAV8, AAV9, AAV10, AAV11, AAV12 or AAV13 and any variants thereof. Typically, the cap gene is cloned downstream of rep so the rep p40 promoter can drive cap expression. This construct does not contain ITRs. This construct is then introduced into a packaging cell line with a second construct containing ITRs, typically flanking a heterologous coding sequence. Helper function or a helper virus are also introduced, and recombinant AAV comprising a capsid generated from capsid proteins expressed from the cap gene, and encapsidating a genome comprising the transgene flanked by the ITRs, is recovered from the supernatant of the packaging cell line. Various types of cells can be used as the packaging cell line. For example, packaging cell lines that can be used include, but are not limited to, HEK293 cells, HeLa cells, and Vero cells, for example as disclosed in US20110201088. The helper functions may be provided by one or more helper plasmids or helper viruses comprising Adenoviral helper genes. Non-limiting examples of the adenoviral helper genes include E1A, E1B, E2A, E4 and VA, which can provide helper functions to AAV packaging. Helper viruses of AAV are known in the art and include, for example, viruses from the family Adenoviridae and the family Herpesviridae. Examples of helper viruses of AAV include, but are not limited to, SadV-13 helper virus and SadV-13-like helper virus described in US20110201088, helper vectors pHELP (Applied Viromics). A skilled artisan will appreciate that any helper virus or helper plasmid of AAV that can provide adequate helper function to AAV can be used herein.

In some instances, rAAV virions are produced using a cell line that stably expresses some of the necessary components for AAV virion production. For example, a plasmid (or multiple plasmids) comprising the nucleic acid containing a cap gene identified as described herein and a rep gene, and a selectable marker, such as a neomycin resistance gene, can be integrated into the genome of a cell (the packaging cells). The packaging cell line can then be transfected with an AAV vector and a helper plasmid or transfected with an AAV vector and co-infected with a helper virus (e.g., adenovirus providing the helper functions). The advantages of this method are that the cells are selectable and are suitable for large-scale production of the recombinant AAV. As another non-limiting example, adenovirus or baculovirus rather than plasmids can be used to introduce the nucleic acid encoding the capsid polypeptide, and optionally the rep gene, into packaging cells. As yet another non-limiting example, the AAV vector is also stably integrated into the DNA of producer cells, and the helper functions can be provided by a wild-type adenovirus to produce the recombinant AAV.

In still further instances, the AAV vectors are produced synthetically, by synthesising AAV capsid proteins and assembling and packaging the capsids in vitro.

Typically, the AAV vectors of the present disclosure also comprise a heterologous coding sequence. The heterologous coding sequence may be operably linked to a promoter to facilitate expression of the sequence. The heterologous coding sequence can encode a peptide or polypeptide, such as a therapeutic peptide or polypeptide, or can encode a polynucleotide or transcript that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous coding sequence and the genomic DNA can occur. As would be appreciated, the nature of the heterologous coding sequence is not essential to the present disclosure. In particular embodiments, the vectors comprising the heterologous coding sequence(s) will be used in gene therapy.

In particular examples, the heterologous coding sequence encodes a peptide or polypeptide, or polynucleotide, whose expression is of therapeutic use, such as, for example, for the treatment of a disease or disorder. For example, expression of a therapeutic peptide or polypeptide may serve to restore or replace the function of the endogenous form of the peptide or polypeptide that is defective (i.e. gene replacement therapy). In other examples, expression of a therapeutic peptide or polypeptide, or polynucleotide, from the heterologous sequence serves to alter the levels and/or activity of one or more other peptides, polypeptides or polynucleotides in the host cell. Thus, according to particular embodiments, the expression of a heterologous coding sequence introduced by a vector described herein into a host cell can be used to provide a therapeutic amount of a peptide, polypeptide or polynucleotide to ameliorate the symptoms of a disease or disorder. In other instance, the heterologous coding sequence is a stretch of nucleic acids that is essentially homologous to a stretch of nucleic acids in the genomic DNA of an animal, such that when the heterologous sequence is introduced into a cell of the animal, homologous recombination between the heterologous coding sequence and the genomic DNA can occur. Accordingly, the introduction of a heterologous sequence by an AAV vector described herein into a host cell can be used to correct mutations in genomic DNA, which in turn can ameliorate the symptoms of a disease or disorder.

In non-limiting examples, the heterologous coding sequence encodes an expression product that, when delivered to a subject using an AAV vector of the present disclosure, treats a disease or condition of the peripheral nervous system (i.e. a disease or condition with a pathology that manifests at least in part in the peripheral nervous system, and/or is caused at least in part by expression of one or more genes in the peripheral nervous system), optionally a disease or condition associated with or characterized by impaired or abnormal Schwann cell function. For example, the disease or condition may be a demyelinating disease or a tumour. In illustrative embodiments, the disease or condition is selected from Charcot-Marie-Tooth disease, Guillain-Barre syndrome, chronic inflammatory demyelinating polyneuropathy (CIDP), neurofibromatosis 1 (NF1), neurofibromatosis 2 (NF2) or Schwannomatosis Those skilled in the art would readily be able to select an appropriate heterologous coding sequence useful for treating such peripheral nervous system-associated diseases and conditions. In some examples, the heterologous coding sequence comprises all or a part of a gene that is associated with the disease, for example in the case of Charcot-Marie-Tooth disease, the gene may be PMP22, GJB1, or SH3TC2. In another example, in the case of NF2 disease, the gene may be MERLIN. In such instances, the AAV vector is typically used for gene replacement therapy in which a functional copy of the gene is introduced, or for genome editing (e.g. CRISPR-Cas9 based genome editing) in which one or more defective copies of a gene is corrected or ablated. In other instances, the heterologous coding sequence encodes a therapeutic protein that is not associated with the disease (i.e. is not causative of the disease in the subject).

The heterologous coding sequence in the AAV vector is flanked by 3′ and 5′ AAV ITRs. AAV ITRs used in the vectors of the disclosure need not have a wild-type nucleotide sequence, and may be altered, e.g., by the insertion, deletion or substitution of nucleotides. Additionally, AAV ITRs may be derived from any of several AAV serotypes, including without limitation, AAV1, AAV2, AAV3, AAV4, AAV5, AAV6, AAV7, AAV8, AAV9, AAV10, AAV11, AAV12 or AAV13. Such ITRs are well known in the art.

As will be appreciated by a skilled artisan, any method suitable for purifying AAV can be used in the embodiments described herein to purify the AAV vectors, and such methods are well known in the art. For example, the AAV vectors can be isolated and purified from packaging cells and/or the supernatant of the packaging cells. In some embodiments, the AAV is purified by separation method using a CsCl or iodixanol gradient centrifugation. In other embodiments, AAV is purified as described in US20020136710 using a solid support that includes a matrix to which an artificial receptor or receptor-like molecule that mediates AAV attachment is immobilized.

### Additional Elements in the Vectors

The vectors of the present disclosure can comprise promoters. In instances where the vector is a nucleic acid vector comprising nucleic acid encoding the capsid polypeptide, the promoter may facilitate expression of the nucleic acid encoding the capsid polypeptide. In instances where the vector is an AAV vector, the promoter may facilitate expression of a heterologous coding sequence, as described above.

In some examples, the promoters are AAV promoters, such as the p5, p19 or p40 promoter. In other examples, the promoters are derived from other sources. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Non-limiting examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system; the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only. In some embodiments, tissue specific promoters are used. Non-limiting examples of such promoters include retina-specific promoters such as human cone L/M-opsin (PR2.1) promoter and variants thereof (see e.g. Ye et al. 2016, Hum Gene Ther 27, 72-82), the chimeric IRBPe/GNAT2 promoter, the synthetic synGNAT2/GNAT2 promoter (Dyka et al. 2014, Adv Exp Med Biol 801, 695-701) and other synthetic retina-specific promoters such as those described by Juttner et al. 2019, Nature Neuroscience 22, 1345-1356). In other embodiments, the promoters selectively function in the brain, and may include, for example, human synapsin 1 (Syn1) promoter, neuron-specific enolase (NSE) promoter, human myelin associated (MAG) promoter (see e.g. Ingusci et al., 2019, Front Pharmacol 10, 724. doi: 10.3389/fphar.2019.00724). The selection of an appropriate promoter is well within the ability of one of ordinary skill in the art.

The vectors can also include transcriptional enhancers, translational signals, and transcriptional and translational termination signals. Examples of transcriptional termination signals include, but are not limited to, polyadenylation signal sequences, such as bovine growth hormone (BGH) poly(A), SV40 late poly(A), rabbit beta-globin (RBG) poly(A), thymidine kinase (TK) poly(A) sequences, and any variants thereof. In some embodiments, the transcriptional termination region is located downstream of the posttranscriptional regulatory element. In some embodiments, the transcriptional termination region is a polyadenylation signal sequence.

The vectors can include various posttranscriptional regulatory elements. In some embodiments, the posttranscriptional regulatory element can be a viral posttranscriptional regulatory element. Non-limiting examples of viral posttranscriptional regulatory element include woodchuck hepatitis virus posttranscriptional regulatory element (WPRE), hepatitis B virus posttranscriptional regulatory element (HBVPRE), RNA transport element, and any variants thereof. The RTE can be a rev response element (RRE), for example, a lentiviral RRE. A non-limiting example is bovine immunodeficiency virus rev response element (RRE). In some embodiments, the RTE is a constitutive transport element (CTE). Examples of CTE include, but are not limited to, Mason-Pfizer Monkey Virus CTE and Avian Leukemia Virus CTE.

A signal peptide sequence can also be included in the vector to provide for secretion of a polypeptide from a mammalian cell. Examples of signal peptides include, but are not limited to, the endogenous signal peptide for HGH and variants thereof; the endogenous signal peptide for interferons and variants thereof, including the signal peptide of type I, II and III interferons and variants thereof; and the endogenous signal peptides for known cytokines and variants thereof, such as the signal peptide of erythropoietin (EPO), insulin, TGF-β1, TNF, IL1-α, and IL1-β, and variants thereof. Typically, the nucleotide sequence of the signal peptide is located immediately upstream of the heterologous sequence (e.g., fused at the 5′ of the coding region of the protein of interest) in the vector.

In further examples, the vectors can contain a regulatory sequence that allows, for example, the translation of multiple proteins from a single mRNA. Non-limiting examples of such regulatory sequences include internal ribosome entry site (IRES) and 2A self-processing sequence, such as a 2A peptide site from foot-and-mouth disease virus (F2A sequence).

### Host Cells

Also provided herein are host cells comprising a nucleic acid molecule or vector (including an AAV vector) of the present disclosure. In some instances, the host cells are used to select, amplify, replicate, package and/or purify a polynucleotide or vector. In other examples, the host cells are used to express a heterologous sequence, such as one packaged within an AAV vector. Exemplary host cells include prokaryotic and eukaryotic cells. In some instances, the host cell is a mammalian host cell, such as a human host cell. It is well within the skill of a skilled artisan to select an appropriate host cell for the expression, amplification, replication, packaging and/or purification of a polynucleotide, vector or rAAV virion of the present disclosure. Exemplary mammalian host cells include cells of the peripheral nervous system, in particular Schwann cells.

### Compositions and Methods

Also provided are compositions comprising the nucleic acid molecules, polypeptides and/or vectors of the present disclosure. In particular examples, provided are pharmaceutical compositions comprising the AAV vectors disclosed herein and a pharmaceutically acceptable carrier. The compositions can also comprise additional ingredients such as diluents, stabilizers, excipients, and adjuvants.

The carriers, diluents and adjuvants can include buffers such as phosphate, citrate, or other organic acids; antioxidants such as ascorbic acid; low molecular weight polypeptides (e.g., less than about 10 residues); proteins such as serum aAAVC.umin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

The AAV vectors of the present disclosure, and compositions containing the AAV vectors, may be used in methods for the introduction of a heterologous coding sequence into a host cell. Such methods involve contacting the host cell with the AAV vector. This may be performed in vitro, ex vivo or in vivo. In particular embodiments, the host cell is a Schwann cell.

When the methods are performed ex vivo or in vivo, typically the introduction of the heterologous sequence into the host cell is for therapeutic purposes, whereby expression of the heterologous sequence results in the treatment of a disease or condition. Thus, the AAV vectors disclosed herein can be administered to a subject (e.g., a human) in need thereof, such as subject with a disease or condition amendable to treatment with a protein, peptide or polynucleotide encoded by a heterologous sequence described herein.

When used in vivo, titres of AAV vectors to be administered to a subject will vary depending on, for example, the particular recombinant virus, the disease or disorder to be treated, the mode of administration, the treatment goal, the individual to be treated, and the cell type(s) being targeted, and can be determined by methods well known to those skilled in the art. Although the exact dosage will be determined on an individual basis, in most cases, typically, recombinant viruses of the present disclosure can be administered to a subject at a dose of between 1×10¹⁰ genome copies of the recombinant virus per kg of the subject and 1×10¹⁴genome copies per kg. In other examples, less than 1×10¹⁰ genome copies may be sufficient for a therapeutic effect. In other examples, more than 1×10¹⁴ genome copies may be required for a therapeutic effect.

The route of the administration is not particularly limited. For example, a therapeutically effective amount of the AAV vector can be administered to the subject via, for example, intravitreal, intrathecal, intravenous, intraperitoneal, subcutaneous, epicutaneous, intradermal, intramuscular, pulmonary, intraosseous, oral, buccal, or nasal routes. In particular embodiments, the AAV vector is administered to the subject via intravitreal injection. The AAV vector can be administrated as a single dose or multiple doses, and at varying intervals.

Also provided are methods for producing an AAV vector described above and herein, i.e. one comprising a capsid polypeptide of the present disclosure. Such methods comprise culturing a host cell comprising a nucleic acid molecule encoding a capsid polypeptide the present disclosure, an AAV rep gene, a heterologous coding sequence flanked by AAV inverted terminal repeats, and helper functions for generating a productive AAV infection, under conditions suitable to facilitate assembly of an AAV vector comprising a capsid comprising a capsid polypeptide of the present disclosure, wherein the capsid encapsidates the heterologous coding sequence.

In order that the invention may be readily understood and put into practical effect, particular preferred embodiments will now be described by way of the following non-limiting examples.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

### Example 1. Experimental Procedures

### Study Design

The objective of this study was to develop novel AAV variants targeting human Schwann cells (SCs) with improved specificity and efficiency. The inventors performed an in vitro selection in primary human SCs (hSCs) using three different libraries, AAV2 and AAV9 peptide-display libraries and a DNA shuffling library, cloned into a functional transduction (FT) platform (see International Patent Application no. PCT/AU2019/051133, the disclosure of which is incorporated herein in its entirety).

Two candidate capsids, Pep2hSC1 and Pep2hSC2, from the AAV2 library screen were selected for further head-to-head comparison with AAV-DJ, which has been previously shown to be the most potent capsid for hSCs, including on primary hSCs, primary fibroblasts, ex vivo human nerve explants and in vivo in mice. Animals were randomly assigned to the individual vectors. Also described herein are four candidate capsids from the AAV9 library screen, Pep9hSC1, Pep9hSC2, Pep9hSC5 and Pep9hSC9, and two candidate capsids from the DNA shuffled library, Shuffle 32 and Shuffle 35.

### Cell Culture Conditions and Cell Origins

AAV production was performed using the human embryonic kidney (HEK) 293T cell line (ATCC) grown in Dulbecco's modified Eagle's medium (DMEM) (Gibco) supplemented with 10% fetal bovine serum (FBS) (Sigma-Aldrich), 1×-penicillin-streptomycin (PS) (Gibco), and 25 mM HEPES (Gibco).

Normal human neonatal dermal fibroblasts (NHDF-neo) used for flow cytometry experiments were kindly provided by Dr Anai Gonzalez-Cordero (CMRI). Cells were cultured and maintained and were grown in growth medium containing DMEM supplemented with 10% FBS, 1⋅×PS, 1×L-Glutamine (Gibco) and 1× non-essential amino acids (Gibco) in a humid 5% CO₂ incubator at 37° C.

### Primary Human Schwann Cells and Fibroblasts

Human tissue was procured, with approval from the local institutional review board, via the Southern Alberta Donation Program and Human Organ Procurement and Exchange Program (Calgary, Canada) with donor or family consent. All samples were non-identifiable to the researchers in the study.

Human skin-derived Schwann cells, dermal fibroblasts and nerve-derived Schwann cells were isolated and purified according to protocol previously published (Chu et al., 2022, Glia 70, 2131-2156). Briefly, for skin-derived Schwann cells, approximately 1 cm² full thickness neck skin was removed from autopsy samples. The skin was washed three times in cold Hank's balanced salt solution (HBSS, Gibco, USA) and then cut into 2-3 mm thin strips and digested in 5 U dispase (Stemcell Tech, Canada) overnight at 4° C. or 2-3 hours at 37° C. The next day, epidermis was removed and discarded and the dermis was cut into 1 mm small pieces, and subsequently placed in 35 mm culture dishes and covered with a minimum volume of DMEM supplemented with 10% FBS and 1×PS, 50 ng/ml human recombinant neuregulin-1 (NRG1, Peprotech, USA), 5 μM forskolin (Fsk, Sigma, USA) and 12.5 μg/mL Plasmocin (Invivogen, USA) for 2 weeks as an explant culture. Media were replaced twice weekly. After 2 weeks, the skin explants were dissociated overnight (37° C. and 5% CO₂) in DMEM with 10% FBS, 1.25 U dispase (StemCell, Canada) and 1.25 mg/ml collagenase IV (Worthington, USA). The next day, the mixture was gently triturated with a 1-ml pipette, the dissociated cells were passed through a 40 μm cell strainer, and centrifuged at 300×g for 6 min at room temperature. After resuspension of the pellet in Schwann cell complete medium containing DMEM supplemented with 10% FBS and 1×PS, 50 ng/ml NRG1, 5 μM Fsk, the cells were plated onto poly-D-lysine and laminin-coated culture dishes for initial expansion.

For nerve-derived SCs, human sciatic nerve samples were obtained from 14 autopsy donors. Individual nerve fascicles were pulled from 1 cm-long sciatic nerve, cut into 3 mm-short segments, and subsequently placed in a 35 mm culture dishes as explant culture described above. Cell dissociation and initial expansion were identical to skin-derived SCs. Samples were then dissociated and cultured as described above.

After a week of expansion, mixed cell populations from both tissue origins were selected against p75 and Thy1 to isolate SCs and dermal fibroblasts, respectively. Hybridoma supernatants from 200-3-G6-4 (ATCC, USA) and K117 (ATCC, USA) were used. Purified cells were further expanded in Schwann cell complete medium before experimentation.

Primary human Schwann cells pn02.8 and pn09.2 were kindly provided by Prof Margaret R Wallace. Media was changed every fourth day for both skin and nerve cells.

For the rat SC cultures, sciatic nerves were harvested from 6-8-weeks-old Wistar rats. All animal care and experimental procedures were approved by the joint CMRI and The Children's Hospital at Westmead Animal Care and Ethics Committee. Nerves were washed three times with ice-cold Hank's Balanced Salt Solution (HBSS) (Sigma). The epineurium was stripped off under a stereomicroscope, and the nerves were washed three times with ice-cold HBSS and were then transferred into a cell culture plate containing, serum-free DMEM. Short segments of 3-5 mm were cut and placed in a 35 mm Petri dish and covered with a minimum volume of DMEM supplemented with 10% FBS and 1×PS, 50 ng/ml NRG1 (Peprotech), 5 μM Fsk (Sigma Aldrich), to keep them attached to the bottom of the dish. For 2 weeks, the medium was replaced twice a week. During this time, the majority of the endoneurial fibroblasts migrate out of the nerve segments onto the culture dish surface, whereas most of the SCs stayed in the nerve segments. After 2 weeks, the nerve segments were dissociated overnight (37° C. and 5% CO2) in DMEM supplemented with 25% dispase (StemCell) and 0.125% collagenase IV (Worthington). The next day, the mixture was gently triturated with a 1-ml pipette, the dissociated cells were passed through a 40 μm cell strainer and centrifuged at 300×g for 6 min at room temperature. After resuspension of the pellet in DMEM supplemented with 10% FCS and 1×PS, 50 ng/ml NRG1, 5 μM Fsk, the cells were plated onto Poly-D-lysine and laminin-coated Petri dishes. SCs purity was expressed as the percentage of cells positive for 5100p, and the total cell number was determined by DAPI staining.

### AAV Library Preparation

The AAV2 peptide display library was generated as previously described (Westhaus et al., 2022, Hum Gene Ther, doi:10.1089/hum.2021.278).

For the AAV9 peptide display library, random heptamers were inserted between amino acids 588 and 589 of the AAV9 VP1 protein. In brief, double SfiI restriction sites were inserted into the local codon-optimized version of the AAV9 cap gene (Caplco9) at the Q588 insertion site. The resulting plasmid pRep2Caplco9_SfiI was then digested with SwaI and NsiI, and the capsid-containing fragment was ligated into SwaI/NsiI digested functional transduction (FT)-spleen focus-forming virus (SFFV) selection platform. This FT-SFFV-Ico9_SfiI construct was subsequently digested twice with SfiI and was dephosphorylated using calf intestine alalkaline phosphatase (NEB), using the manufacturer's protocol. The 7-mer random inserts were made double-stranded using a short primer binding on the homology arm upstream of the peptide (dslco9-library). The final library was generated by mixing 225 fmol of the digested FT-SFFV-Ico9_SfiI backbone with 2,250 fmol of the dslco9-library insert into individual NEBuilder (NEB) reactions. The reactions were combined after assembly and purified using ethanol precipitation. The resulting pellet (1 μg of DNA) was used for electroporation into SS320 competent cells (Lucigen). The recovered transformants were used to inoculate 250 mL of lysogeny broth (LB) containing 10 μg/mL trimethoprim (TMP). Only 10 μL of recovered transformants were used to plate a five 10-fold dilution series of the electroporated bacteria to determine transformation efficiency. Total FT-SFFV-Ico9_7mer library plasmid was purified with an EndoFree Maxiprep Kit (Invitrogen).

For the Shuffled AAVLib_1-12 capsid plasmid library, the AAV library was generated as previously described (Cabanes-Creus et al., 2019, Mol Ther Methods Clin Dev 12, 71-84). AAV variants 1 to 12 were included in the parental mix. To move the same library into the FT platform, the pRC-AAVLib_1-12 was digested overnight alongside the pFT-SFFV platform with SwaI and NsiI. 1.4 μg of the insert was ligated at 16° C. with T4 DNA ligase (NEB) for 16 h into 1 μg of the pFT-SFFV platform. Ligation reactions were concentrated by using ethanol precipitation, electroporated into SS320 electro-competent bacteria, and grown as described above.

### AAV Library Selection

The screening of AAV variants in cultured primary human SCs was based on the functional transduction (FT)-based method (Westhaus et al., 2022, Hum Gene Ther, doi:10.1089/hum.2021.278). The Lco2 capsid variants were selected by screening a Ico2 peptide display library on hSCs in vitro. Approximately 2×10¹⁰ vector genomes (vgs) of library were used to transduce the cultured primary hSCs for 24 h. 7 days after transduction, DNA and RNA were isolated from cell pellets. Capsid variants DNA and expressed mRNA were amplified from the DNA and cDNA samples, respectively, with primers Ico2_PepLibF/PepLib-R surrounding the 7-mer insert. The amplicon was used for Gibson assembly into the twice SfI-digested FT-SFFV-Ico2 library recipient plasmid and electroporated into bacteria, as described in the AAV library preparation section above. The peptides recovered from the first round of screening were packaged and screened again in the cultured primary hSCs. Based on the NGS results, the recovered variants were ranked, and the top candidates were picked as described below and respective nucleotide sequences encoding these peptides were cloned into the cap gene of Ico2 to obtain helper plasmids for producing capsid variants as vectors. Similar screening methods were performed with the Ico9-based peptide display library. Briefly, the Ico9-based peptide display was analyzed using NGS at every step of selection, including before selection (packaged library), after round 1 (FT-RNA), and after round 2 (FT-RNA), using primers Ico9_PepLib_F/PepLib-R. The recovered variants were ranked and the top candidates were selected for further tests as described below.

For the shuffled AAVLib1-12 capsid library, selected AAV cap genes were recovered after four rounds of iterative passage in SCs with flanking primers (F/R-cap-recovery). Briefly, genomic DNA (gDNA) was extracted, and the cap sequences were amplified by PCR using the F/R-cap-recovery primers (Table 2) and cloned directly with Gibson assembly into recipient plasmid. The reaction was used for electroporation and grown as above. The full cap was then excised using SwaI and NsiI and cloned into the FT-SFFV platform with 20 individual ligations at 16° C. with T4 DNA ligase overnight. The ligations were combined and purified using ethanol precipitation. The resulting pellet (1 μg of DNA) was used for electroporation into competent cells. AAV capsid ORFs from round 4 were cloned into standard packaging plasmid harboring rep2 with Gibson assembly and 50 randomly chosen clones were sent for full Sanger sequencing with primers External_Seq_F/R and internal_cap_Seq (Table 2).

TABLE 2
Oligonucleotide sequences

| Primer | | |
|---|---|---|
| name | Sequence (5′→3′) | SEQ ID NO: |
| Pep2hSC1-F | GATTCGTGGTCTGAGGCGGCCGCGACCGCA | 28 |
| Pep2hSC1-R | ACCCCGCTTCCTCTGGCCCTGGAGGTTGGT | 29 |
| Pep2hSC2-F | GTTGATTTTGATAAGGCGGCCGCGACCGCA | 30 |
| Pep2hSC2-R | ATCCTTCCTCCTCTGGCCCTGGAGGTTGGT | 31 |
| Pep9hSC1-F | GACGTGCGCGTCCAGGCGGCCCAAACCGGA | 32 |
| Pep9hSC1-R | GCCCCTCGCACTCTGGCCCTGGTGGTTGGT | 33 |
| Pep9hSC2-F | GATGTGGCTGGGGAGGCGGCCCAAACCGGA | 34 |
| Pep9hSC2-R | ACCCCGACCCCTCTGGCCCTGGTGGTTGGT | 35 |
| Pep9hSC5-F | CGTCCTCCTCCTCAGGCGGCCCAAACCGGA | 36 |
| Pep9hSC5-R | ATCAGCACGACTCTGGCCCTGGTGGTTGGT | 37 |
| Pep9hSC9-F | GACATGCTCGGGAAGGCGGCCCAAACCGGA | 38 |
| Pep9hSC9-R | GCCCCGCGCACTCTGGCCCTGGTGGTTGGT | 39 |

### AAV Packaging

All AAV vectors were produced in HEK293T cells by triple-transfection using PEI MAX (Polysciences) to package two unique single-stranded ITR2-CMV-eGFP-N₆Barcode(BC)-WPRE-ITR2 transgenes each (see Westhaus et al., 2020, Hum Gene Ther 31, 575-589). The FT-Ico2_7mer, FT-SFFV-Ico9_7mer and FT-SFFV-AAVLib_1-12 libraries (alongside pRep2 helper plasmids) were produced in ten 15 cm dishes of HEK293T cells. For the CMV-GFP-N₆BC-WPRE construct, 5 μg of transgene plasmid was transfected per 15 cm dish, while to reduce cross-packaging, 400 ng of transgene plasmid was transfected per 15 cm dish for library production. Three days post-transfection, recombinant virus was harvested from the cells and media, and purified by ultracentrifuge using iodixanol gradient as previously described (Khan et al., 2011, Nat Prot 6, 482-501). AAV titers were quantified by droplet digital PCR (ddPCR) (Bio-Rad, Berkeley, CA, USA) using EvaGreen supermix (Bio-Rad) and following the manufacturer's instructions using eGFP-specific primers (Table 2, above).

### Mouse Studies

All animal care and experimental procedures were approved by the joint Children's Medical Research Institute and The Children's Hospital at Westmead Animal Care and Ethics Committee. For animals receiving intraneural AAV-CMV-eGFP, in-house bred 16 weeks-old male C57BL/6J mice were used. Protocols were approved by the Animal Care Committee at the University of Calgary. All applicable international, national, and institutional guidelines for the care and use of animals were followed. Briefly, mice were deeply anesthetized using 2% isoflurane with oxygen, the surgical area was shaved and disinfected with isopropanol and betadine. Buprenorphine (0.05 mg/kg, s.c.) was given 15 minutes prior to surgery. Sciatic nerve was exposed at mid-thigh level and crushed twice with a pair of number 5 forceps for 10 seconds each. The nerve was then injected with 3 ul of AAVs, AAV-DJ, Pep2hSC1 or Pep2hSC2 using a 5 μl Hamilton syringe fitted with a 33 gauge needle. The crush site was landmarked with a 10-0 suture. The AAVs were delivered proximal as well as distal to the crushed site to determine the susceptibility of normal and injured Schwann cells. After the injection, the wound was closed with 6-0 prolene sutures and animals returned to their home cages on a heating pad for recovery. Analgesics Metacam (1 mg/kg, s.c.) was given once daily for 2 days after surgery. Nerves were harvested one month after surgery. Briefly, animals were deeply anesthetized with 5% isoflurane followed by intraperitoneal injection of overdosed sodium pentobarbital. Sciatic nerve was re-exposed and harvested and processed as described below.

FRG mice were housed in individually ventilated cages with 2-(2-nitro-4-trifluoro-methylbenzoyl)-1,3-cyclohexanedione (NTBC) supplemented in drinking water (8 mg/ml). FRG mice, 6 to 8 weeks old, were engrafted with human hepatocytes (Lonza Group Ltd., Basel, Switzerland) as described previously (Azuma et al., 2007, Nat Biotechnol 25, 903-910). Levels of human hepatocyte engraftment in chimeric mice were estimated by measuring the presence of human albumin in peripheral blood, using the human albumin ELISA quantitation kit (Bethyl Laboratories). hFRG mice were placed on 10% NTBC before transduction with vectors and were maintained on 10% NTBC until harvest. 2×10¹¹ vgs of each AAV variant were injected intravenously (lateral tail vein) into hFRG mice. Mice were euthanized 2 weeks post injection, with one liver lobe collected for IHC analysis prior to liver perfusion.

Hepatocytes for flow cytometry analysis were obtained by collagenase perfusion of the liver as previously described (Cabanes-Creus et al., 2020, Sci Transl Med 12, 1-13) with minor modifications. To distinguish between mouse liver cells and human hepatocytes, cells were labeled with biotin-conjugated anti-human-HLA-ABC (eBioscience; 1:100), phycoerythrin-conjugated anti-mouse-H-2K^{b} (BD Pharmigen; 1:100), and allophycocyanin-conjugated streptavidin (eBioscience; 1:500). GFP-positive-labeled samples were sorted to a minimal 95% purity using a BD AriaIII cell sorter. Flow cytometry was performed in the Flow Cytometry Facility, Westmead Institute for Medical Research, Westmead, NSW, Australia. The data were analyzed using FlowJo 7.6.1 (FlowJo LLC).

DNA and RNA Extraction from Cells

DNA and RNA were isolated from the cell pellets from the in vitro experiments using the AllPrep DNA/RNA Mini Kit (Qiagen) following the manufacturer's instructions.

DNA Isolation from Human Hepatocytes

Isolation of DNA was performed, as described previously (Westhaus et al., 2020, Hum Gene Ther 31, 575-589) without modifications. Briefly, DNA was extracted using a standard phenol:chloroform protocol after proteinase K digest and rNase A digestion step.

### Reverse Transcription of Extracted RNA

Five hundred nanograms of total RNA was treated with TURBO dNase (Invitrogen) following the manufacturer's instructions. The dNase-treated RNA was then used for cDNA synthesis using SuperScript IV First-Strand Synthesis System (Invitrogen) following the manufacturer's instructions using 2 μM of a WPRE-binding primer (see Table 2 above) to specifically synthesize AAV encoded transgene cDNA for barcoded NGS analysis or 2 μM of local codon-optimized AAV capsid reverse primer (Ico2/Ico9-NGS R) for peptide coding region recovery in the FT-RNA library selection.

### Barcode Amplification, NGS, and Distribution Analysis

Isolation of DNA and RNA and cDNA synthesis was performed, as described previously (Cabanes-Creus et al., 2022, Mol Ther Methods Clin Dev 24, 88-101) without modifications. Briefly, the barcoded region was amplified from 50 ng of extracted total genomic and vector DNA as well as 3 μL final cDNA product with 1 of 5 BC_F forward primers (barcoded to allow multiplexing of different samples) and the universal reverse BC_R primers using the Q5 high-fidelity DNA polymerase (NEB). NGS reads from the DNA and cDNA populations were normalized to the reads from the respective mixes of the vectors. Heatmaps were generated using the ‘pHeatmap’ (version 1.0.12) source code in R Studio. DNA/cDNA NGS reads were normalised to the NGS reads prior to injection and scaled using the ‘scale’ function in R Studio. Clustering was performed according to the Euclidean distance between scaled values.

### Vector DNA Copy Number Per Cell

Vector copy numbers were measured via ddPCR using EvaGreen supermix and following the manufacturer's instructions. To detect AAV genomes, eGFP primers were used, and vector genomes were normalized to either human or mouse albumin copy number using primers hALB_F/R for cells from human origin as well as mALB_F/R for cells from mouse origin (see Table 2 above).

### Heparin Competition Assay on HEK293T Cells

1.5×10⁵ HEK293T cells per well (24-well format) were seeded 24 h before transduction in culture medium (DMEM supplemented with 10% FBS and 1% P/S). Cells were transduced with or without in the presence of 500 mg/mL heparin. Percentage of transgene-expressing cells was determined by flow cytometry 72 h post-transduction.

### Neutralization Assay

Briefly, 1.5×10⁵ HEK293T cells per well (24-well format) were seeded 24 h before transduction. Vectors diluted in DMEM medium (supplemented with 10% FBS and 1% P/S) were incubated for 1 h at 37° C. with undiluted (neat), 1:2 diluted, 1:4 diluted, 1:16 diluted, and 1:32 diluted human IVIg (Intragam® 10, 10 g/100 mL, CSL Behring). The mixture of vector and serum diluted in medium was added onto the cells and incubated for 48 h at 37° C. and 5% C02. Transduction efficiency in the presence of IVIg was normalized to the vector performance in the absence of IVIg (performed in parallel) and was analyzed by flow cytometry by determining the percentage of eGFP-positive cells.

### In Vitro AAV Transduction and NGS

Mouse, rat or human SCs were seeded on a poly-D-lysine and laminin-coated 6-well plate using the culture conditions indicated above. Fibroblast were seeded at 300,000 cells per well in 6-well plates. The cells were incubated overnight with the ‘AAV Testing Kit’ or the ‘hSC Testing Kit’. After PBS wash, fresh medium was provided, and all cells were allowed to grow for an additional 32 h before harvest. Cells were harvested by incubating with TrypLE Express (Gibco) for 5 min at 37° C. The cells were then recovered in fresh media, spun at 300×g for 5 min, and the dry pellet was used for DNA and RNA extraction.

### In Vitro AAV Transduction and Flow Cytometry

Mouse, rat or human SCs were seeded on a poly-D-lysine and laminin-coated 12-well plate using the culture conditions indicated above. Fibroblast were seeded at 300,000 cells per well in 6-well plates. The cells were incubated overnight with respective AAV vector variants at a multiplicity of transduction (MOT) of 1,000 vg per cell.

After 16 h, cells were then washed, and media were then replaced. 3 days after exposure to the vector, the cells were rinsed once with⋅PBS (Gibco), dissociated using TrypLE Express, and recovered in their culture media. Following transfer into 5 mL polystyrene tubes, the cells were spun down at 300× g for 5 min and resuspended in FACS buffer (PBS, 2% FBS, 5 mM EDTA (Invitrogen). The flow cytometry analysis was performed using a BD FACSCanto cell analyzer. The flow cytometry data was analyzed with Flow Jo 7.6.1.

### AAV Transduction of Primary SC Cultures and Immunofluorescence

Rat or human SCs were seeded into 24-well containing poly-D-lysine and laminin-coated coverslips plates and allowed to adhere overnight. SCs were transduced with rAAVs packaged using candidate capsids at a MOT of 1,000. After 16 h, cells were then washed, and media were then replaced. eGFP expression was assessed by fluorescence microscopy (Zeiss Axio Imager.M1) 3 days after AAV exposure.

### AAV Transduction of Human Nerve Explants and Immunofluorescence

For the human nerve explants, individual nerve fascicles were pulled from autopsied sciatic nerve and cut into 1 cm nerve segments. AAV diluted in PBS/5% sucrose (1.1×10¹⁰ GC, with the exception of AAV2.7m8 and AAV-Shuffle35 for which 1.1×10⁹ GC were used) was injected into the fascicles using a 5 μl Hamilton syringe fitted with a 33 gauge needle, the nerve was trimmed to 0.5 cm segment with maximal bolus retention. Individual nerve explants were then placed separately in 24-well plates for 14 days in DMEM with 10% FBS, 1% P/S supplemented with 50 ng/ml NRG1 and 5 μM Fsk, in a humidified incubator at 5% CO₂ and 37° C. Media were changed twice weekly.

### Immunohistochemistry

For immunostaining of chimeric liver samples, one lobe was collected and was fixed with 4% (w/v in PBS) paraformaldehyde (PFA) overnight at 4° C. before being cryoprotected through a sucrose gradient (10%, 20% and 30% w/v sucrose in PBS). Liver samples were then frozen in optimal cutting temperature O.C.T. (Tissue-Tek; Sakura Finetek USA, Torrance, CA). Liver sections (5 μm) were prepared on a Cryostat (Leica, cat #CM1950). Sections were permeabilized with 0.1% Triton X-100 in PBS (PBST) and blocked in 10% Donkey serum (Sigma Aldrich), 9% FBS in PBS for 1 h at room temperature.

Sections were then incubated with rabbit monoclonal anti-human GAPDH antibody conjugated with Alexa Fluor 647 (Abcam, clone AF674, 1:600 dilution) at room temperature for 2 h. Histological analysis using immunofluorescence microscopy was performed using a Zeiss Axio Imager.M1 with ZEN 2 software.

For immunostaining of the sciatic nerve, sciatic nerve was dissected, immerse-fixed overnight in 4% PFA in 0.1M phosphate buffer (PB) at 4° C. The specimens were then cryoprotected in 30% sucrose in PB solution at 4° C. until the specimens had sunk. They were then embedded in O.C.T. and sectioned on a cryostat at a thickness of 10 μm. Sections were collected on SuperFrost Plus slides (VWR, USA). Sections were permeabilized and blocked with 10% normal serum, in PBST solution containing 0.3% Triton X-100 for 1 h at room temperature. Sections were then incubated with primary antibodies including rabbit anti-CD271 (p75ntr) (BioLegend, clone Poly18397, 1:1,000), rabbit anti-Neurofilament 200 (Sigma N4142, 1:500), mouse anti-PRX (Novus, USA, 1:500) and rabbit anti-S10013 (Dako, USA, 1:1000) in diluent at 4° C. overnight. Sections were washed in PBST buffer, then labelled with corresponding secondary antibodies for 1 h at room temperature. After washing with PBS, sections were mounted with PermaFluor mounting medium (Thermo Scientific, USA), and imaged with a slide scanner (VS110, Olympus, Japan).

For cultured SCs, cells were first washed in cold PBS and then fixed in 4% PFA for 10-15 min at 25° C. After washing three times in PBS, cells were permeabilized in 0.2% PBST and blocked in 10% goat serum in PBST at 25° C. for 30 min. Staining was performed with a FLEX rabbit polyclonal S100 antibody (Dako) overnight at 4° C. Cells were washed three times in PBS and incubated with a secondary antibody Alexa Fluor Plus 594 (Thermo Fisher, 1:500) at 25° C. for 1 h. Cells were then washed in PBS and counterstained with DAPI (Invitrogen) at 0.08 ng/mL.

### Statistics and Reproducibility

All data are presented as mean±SEM. Statistical significance was assessed using Graphpad Prism 9 software. A p value of 0.05 or less was considered significant in all experiments.

### Example 2. Identifying Capsids with Improved Efficacy on Primary SCs Using the FT Platform

Previously undescribed AAV capsids that, when vectorised, have the ability to efficiently transduce Schwann cells were identified in the studies described herein.

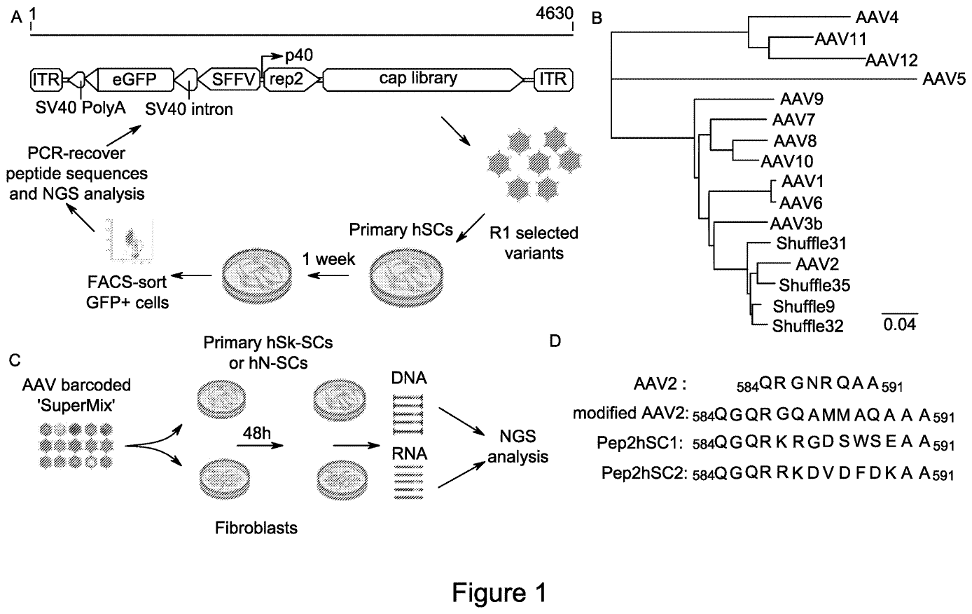

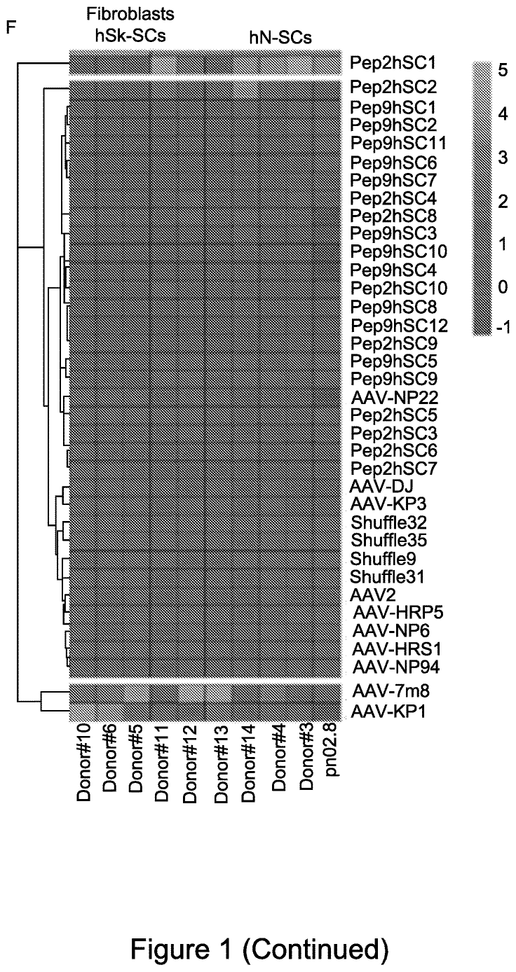

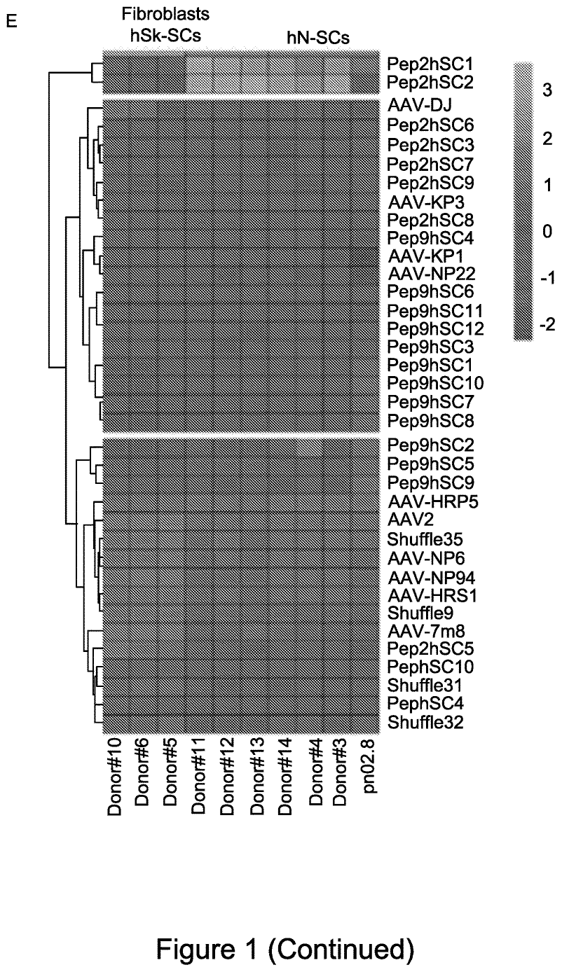

To identify novel variants of AAV vectors for efficient transgene delivery to hSCs, the inventors performed AAV-directed evolution selections on cultured primary hSCs. Three bespoke capsid libraries were generated: AAV2 and AAV9 peptide display libraries, as well as a DNA shuffled library based on the capsid genes from AAV serotypes 1 to 12. The three libraries were cloned into the proprietary FT library constructs, which allows for efficient variant selection based on transgene expression at the RNA level (see Westhaus et al., 2022, Hum Gene Ther, doi:10.1089/hum.2021.278). Following packaging, all three AAV vector libraries were used to transduce primary hSC cultures. To increase both stringency and the chance of selecting the most functional variants, transduced cells underwent fluorescence-activated cell sorting (FACS) based on the expression of FT library-encoded eGFP reporter (FIG. 1A). eGFP positive cells were subsequently used for RNA extraction and PCR recovery of most functional variants from AAV-encoded RNA/cDNA.

After two rounds of selection, the inventors selected the 10 top variants from the AAV2 peptide display library, namely Pep2hSC1-Pep2hSC10. Interestingly, the inventors were able to detect only a very small number of eGFP positive hSCs for the other two libraries (AAV9 peptide display and the shuffled library, data not shown) and thus opted to process the entire hSC population for capsid recovery. After two rounds of selection of the AAV9 peptide display library 12 highly enriched capsids (Pep9hSC1-Pep9hSC12) where identified in the RNA/cDNA extracted from the whole SC population. The DNA shuffled library underwent four rounds of selection. After sequencing 15 random clones we identified four capsid (Shuffle9, 31, 32, and 35) variants from a phylogenetically distinct subpopulation closely related to AAV2 (FIG. 1B). The four shuffled capsids were included in subsequent functional evaluations.

The inventors next performed a functional evaluation of the selected 26 novel variants on hSCs. As reference controls, 10 previously published natural and bioengineered AAV variants that were identified using the inventors' published ‘AAV Testing Kit’ approach (Westhaus et al., 2020, Hum Gene Ther 31, 575-589) for their ability to functionally transduce primary hSCs in culture were used. Specifically, two single-barcoded AAV expression cassettes encoding eGFP fluorescent reporter under the control of the ubiquitous human cytomegalovirus (CMV) immediate-early enhancer and promoter were packaged (see Westhaus et al., 2020, Hum Gene Ther 31, 575-589) per novel variant and reference control. Vectors were individually titrated and pooled at an equimolar ratio. The barcoded “‘hSC Testing Kit’” was tested on cultured primary hSCs isolated from the skin (hSk-SCs) or from the sciatic nerve (hN-SCs) from multiple donors (Chu et al., 2022, Glia 70, 2131-2156). Human dermal fibroblasts were also transduced in parallel to confirm the specificity of the capsids for targeting only the hSCs. After 48 hours, cells were harvested, and DNA and RNA extracted for subsequent NGS analysis of the barcode composition at the DNA (cell entry) and RNA/cDNA (transgene expression) levels as per the method of Westhaus et al., 2020, Hum Gene Ther 31, 575-589 (FIG. 1C).

For viral entry (DNA biodistribution) (FIG. 1E), the analysis identified two variants, RKRGDSWSE and RRKDVDFDK referred to as Pep2hSC1 and Pep2hSC2 respectively (FIG. 1D), with relatively high enrichment in both hSk-SCs and hN-SCs, which were almost absent in fibroblasts. For transgene expression (RNA/cDNA) (FIG. 1F), Pep2hSC1, and to a lower extent Pep2hSC2, were the most enriched in both hSk-SCs and hN-SCs. Both variants also demonstrated high specificity for SCs, unlike AAV2.7m8, which performed very well regardless of the target cell type. Finally, AAV-DJ, which has been previously reported to be the most efficient at transducing hSCs (Bai et al., 2019, Gene Ther, doi:10.1038/s41434-019-0080-9) was outperformed by AAV2.7m8 and the novel variants.

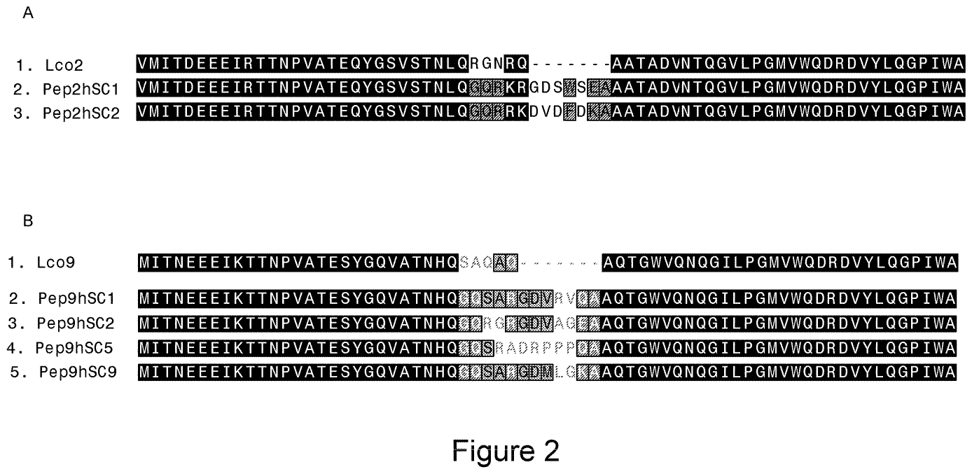

The amino acid sequences of the variant capsids disclosed herein are provided at the end of the specification. The regions of the variant capsids derived from the AAV2 and AAV9 libraries that differ from the relevant parental AAV2 or AAV9 capsid (also referred to herein as the 12 amino acid substitution or peptide modification) are shown in Table 3 below, and alignments of the regions of the capsids including the variable sequences are shown in FIG. 2.

TABLE 3

| Capsid | Variant amino | |
|---|---|---|
| variant | acid sequence | SEQ ID NO: |
| Pep2hSC1 | GQRKRGDSWSEA¹ | 4 |
| Pep2hSC2 | GQRRKDVDFDKA¹ | 5 |
| Pep9hSC1 | GQSARGDVRVQA² | 10 |
| Pep9hSC2 | GQRGRGDVAGEA² | 11 |
| Pep9hSC5 | GQSRADRPPPQA² | 12 |
| Pep9hSC9 | GQSARGDMLGKA² | 13 |

¹12 amino acid substitution (peptide modification) relative to AAV2 capsid, replacing amino acids at positions 585-589 of AAV2 (SEQ ID NO: 1)
²12 amino acid substitution (peptide modification) relative to AAV9 capsid, replacing amino acids at positions 586-590 of AAV9 (SEQ ID NO:2)

To evaluate if the Pep2hSC1 and Pep2hSC2 variants selected in primary hSCs could also transduce primary cells from other species, the inventors performed the same AAV barcoded “‘hSC Testing Kit’ analysis on primary rat and mouse SCs (data not shown). The bioengineered AAV-HRP5, a variant previously selected for superior homologous recombination in HuH-7 cells (Cabanes-Creus, 2019, PhD Thesis), was the top performer both at cell entry (DNA) and transgene expression (RNA/cDNA) levels in rat cells while AAV-KP1 (Pekrun et al., 2019, JCI Insight 4, doi:10.1172/jci.insight.131610) was the top variant in mouse SCs. Interestingly, Pep2hSC1 and Pep2hSC2, efficiently transduced primary rat SCs with 60% and 40% of eGFP positive cells respectively. AAV2.7m8 did not transduce rat SCs efficiently (17%). Pep2hSC1 and Pep2hSC2 were able to transduce primary mouse SCs. However, eGFP expression was also observed in non-SCs cells (difference in morphology) following Pep2hSC1 transduction, while eGFP expression was strictly limited to SCs with Pep2hSC2.

### Example 3. Characterisation of Pep2hSC1 and Pep2hSC2

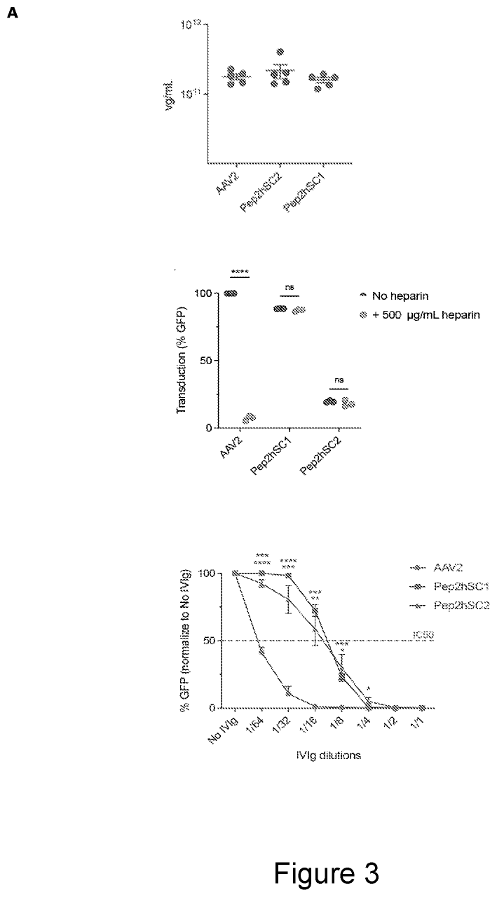

For further characterization, the inventors decided to focus on two variants, Pep2hSC1 and Pep2hSC2. As manufacturability is one of the biggest bottlenecks impacting clinical development of many novel bioengineered variants, the production yields of the variants was first evaluated using standard adherent HEK293T production protocols. The data showed that there was no significant difference in manufacturability between the novel variants and the parental AAV2 capsid (FIG. 3A). As clinical-grade AAV2 has been produced at scale to support clinical studies, the data indicate that the novel SC-tropic variants would be amenable to large scale production using standard AAV manufacturing protocols.

Based on the relative low performance of both Pep2hSC1 and Pep2hSC2 compared to AAV2 and other variants that harbor the heparin-binding motif of AAV2 (AAV-HRS1, AAV-HRP5, AAV-NP6, AAV-NP94 and the Shuffled variants) in immortalized hSCs, and the fact that peptide insertions at position 587-588 of AAV2 capsid sequence are known to reduce the heparan sulfate proteoglycan (HSPG) binding phenotype (Opie et al., 2003, J Virol 77, 6995-7006; Kern et al., 2003, J Virol 77, 11072-11081), the inventors hypothesised that Pep2hSC1 and Pep2hSC2 were HSPG de-targeted and their transduction was not dependent on heparan sulfate binding. To test this, a heparin competition assay was performed. As expected, the transduction efficiency of AAV2 was significantly reduced by soluble heparin. However, the performance of Pep2hSC1 and Pep2hSC2 was unaffected (FIG. 3B) supporting the hypothesis regarding the heparin sulfate binding of the new variants.

It is known that peptide insertions at this location not only alter vector tropism but also impact the structure of the most protruding surface exposed loop. This modified structure had the potential to impact recognition by pre-existing neutralising antibodies. The inventors therefore performed a neutralisation assay with serial dilutions of human intravenous immune globulin (IVIg). Both novel variants demonstrated significantly improved immune escape properties compared to the parental AAV2 capsid (FIG. 3C).

### Example 4. Novel Variants Show a Strong Tropism for Primary hSCs

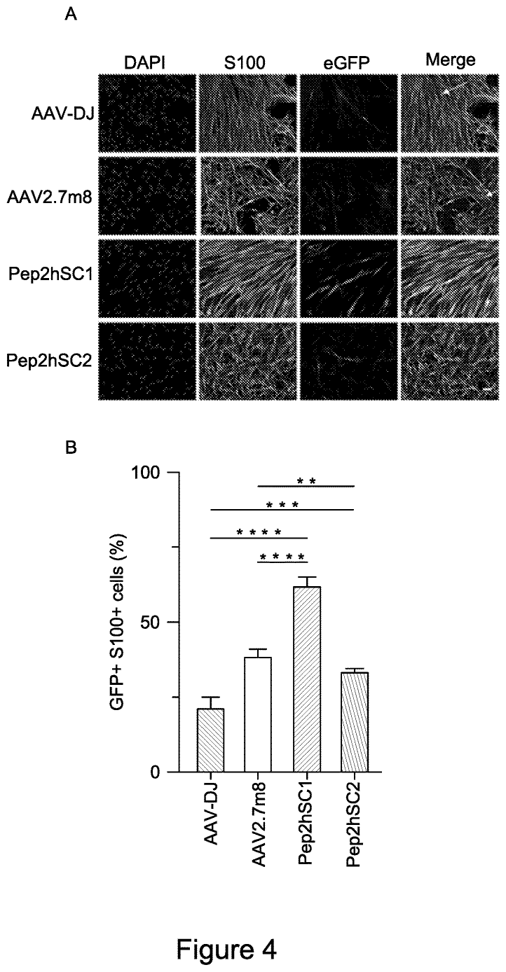

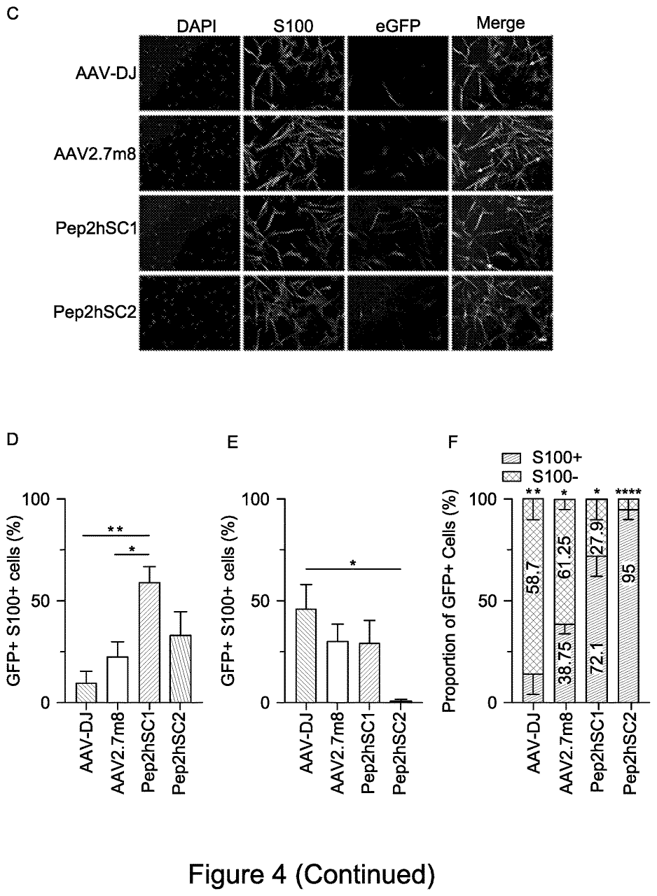

The inventors next performed a detailed functional evaluation of the Pep2hSC1 and Pep2hSC2 variants on primary hSCs compared to control capsid AAV-DJ, which has been reported to efficiently transduce hSCs (Bai et al., 2019, Gene Ther, doi:10.1038/s41434-019-0080-9). AAV2.7m8 was also included which, based on the present study (see FIG. 1E-F), was highly efficient at transgene delivery to hSCs. Primary hSCs were transduced using a low multiplicity of transduction (MOT) of 1,000 vg/cell and harvested three days later. Transduction efficiency was then determined by immunofluorescence (FIG. 4A and C) and by flow cytometry three days later. Data supported the NGS results (FIG. 1E-F) and showed that the two novel AAV variants, Pep2hSC1 and Pep2hSC2, transduced hSCs with higher efficiency than both AAV-DJ and AAV2.7m8 (FIG. 4A-B).

Next, the inventors determined the specificity of the Pep2hSC1 and Pep2hSC2 variants at transducing primary hSCs, using the pn09.2 culture, which contained both S100+(~60%) and S100− cell populations (~40%) as demonstrated by colocalization with the SCs marker S100P. Cells were transduced as outlined in FIG. 3A-B. Quantification of the eGFP signal in the S100+ population three days post-transduction showed that both Pep2hSC1 and Pep2hSC2 had greater efficiency at functionally transducing hSCs than AAV-DJ and AAV2.7m8 (FIG. 4D). Furthermore, while Pep2hSC1 transduced the S100− cell population at similar efficiency to AAV2.7m8, Pep2hSC2 did not transduce S100− cells at a detectable level (FIG. 4E-F). The inventors repeated the experiment using a higher MOT of 10,000 vg/cell and we observed a similar trend, with Pep2hSC1 and Pep2hSC2 transducing over 75% of hSCs (S100+) and Pep2hSC2 not transducing S100− cells (data not shown). To gain additional insights into the function of Pep2hSC2, we transduced fibroblast cultures with higher vector dose. Remarkably, Pep2hSC2 did not transduce fibroblasts even when the highest MOT was used (data not shown). Finally, by plotting the data as the percentage of the total transduced eGFP+ cells, Pep2hSC1 and Pep2hSC2 clearly showed a preferential tropism to SCs (S100+) over the S100− population (FIG. 4F). Conversely, AAV-DJ and AAV2.7m8 displayed a stronger preference for transduction of fibroblasts (S100−) over SCs (S100+) (FIG. 4F) at MOT of 1,000 vg/cell and this preference towards fibroblasts was further enhanced at MOT 10,000 vg/cell (data not shown).

Recognizing the role of SCs in disorders like neurofibromatosis, particularly in the formation of plexiform neurofibromas, the inventors evaluated the performance of Pep2hSC1 and Pep2hSC2 in primary hSCs isolated from a plexiform neurofibroma, namely pNF01.3. This cell line harbours a mutation in exon 5 (c.565A>T, 787T) of the NF1 gene. The culture contained approximately 90% SCs (S100+). The pNF01.3 SC culture was transduced at MOTs of 1,000 and 10,000 vg/cell and the eGFP signal analyzed by immunofluorescence three days post-transduction.

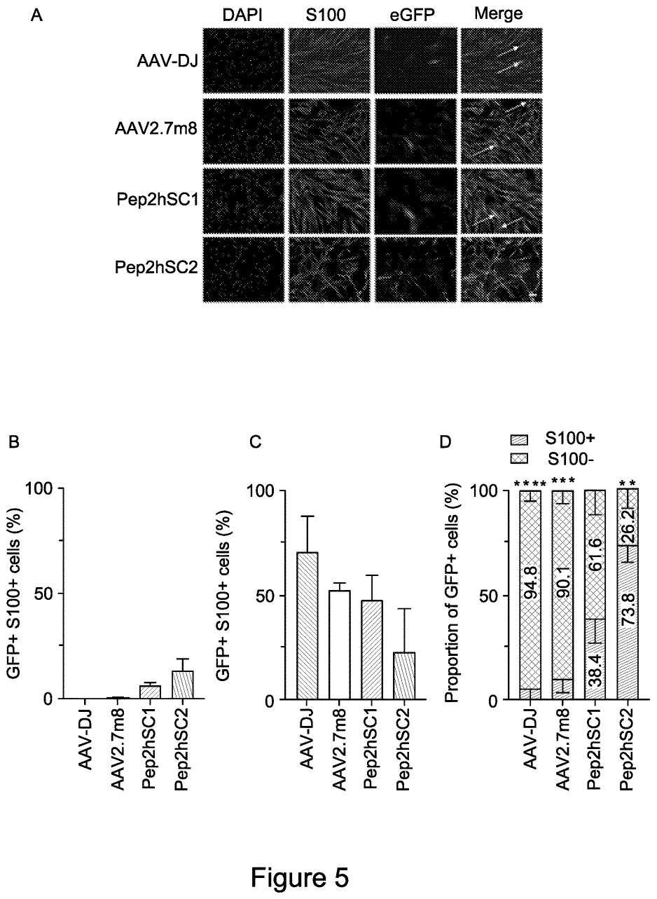

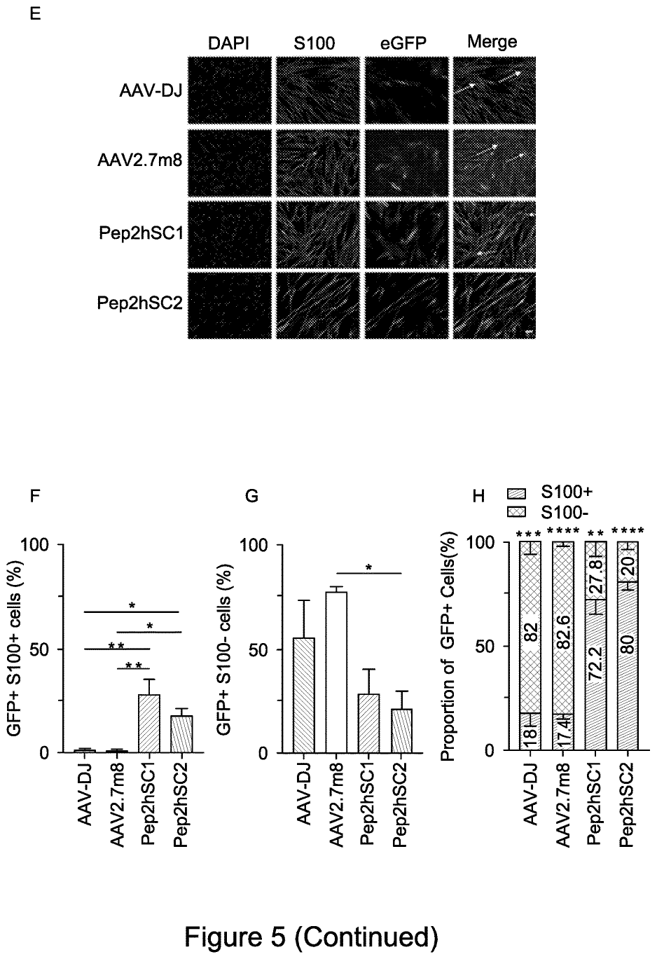

The transduction levels of the four AAV variants in pNF01.3 culture were lower than those observed in previous experiments involving “healthy” hSCs isolated from nerve tissue. At MOT of 1,000 vg/cell, Pep2hSC1 and Pep2hSC2 showed higher transduction in S100+ cells, at 6% and 13.4% respectively. In contrast, AAV-DJ and AAV2.7m8 exhibited minimal transduction with no detectable eGFP fluorescence in these cells (FIG. 5A and FIG. 5B). The Pep2hSC1 and Pep2hSC2 variants demonstrated the strongest performance in hSCs isolated from a plexiform neurofibroma among the AAVs tested.

Interestingly, in the S100− cell population, Pep2hSC1, AAV-DJ, and AAV2.7m8 achieved relatively high transduction efficiencies (70%, 52%, and 48%, respectively), while Pep2hSC2 transduced less than 25% of the cells (FIG. 5C). When the MOT was increased to 10,000 vg/cell, the pattern remained consistent, with negligible transduction for AAV-DJ and AAV2.7m8. However, Pep2hSC1 and Pep2hSC2 showed improved transduction in S100+ cells by 4.6-fold and 1.4-fold, respectively (FIG. 5F). Notably, the enhanced transduction efficiency of S100+ cells with Pep2hSC1 was accompanied by an increased transduction rate in S100− cells (77%), while Pep2hSC2 showed no significant difference in this group (less than 25%) (FIG. 5G). Finally, both Pep2hSC1 and Pep2hSC2 display a preferential tropism for SCs (S100+) compared to the S100− population even at the higher MOTs tested (FIGS. 5D and 5H).

Together these results demonstrate that Pep2hSC1 and Pep2hSC2 display superior performance over AAV-DJ and AAV2.7m8 in primary hSCs derived from healthy donors and a NF1 patient. Furthermore, Pep2hSC2 exhibited an improved specificity for the SCs, with almost no transduction of fibroblasts.

### Example 5. Evaluation of Novel Capsids for Transduction Efficiency of Human Nerve Segments

Assessment of AAV tropism in cell cultures, while highly informative, has its limitations since the SCs in culture do not exhibit the same structures and differentiation as SCs surrounding neuronal axons. Indeed, it has been shown previously that the transduction of monolayers of cultured SCs does not predict the transduction efficiency in nerve segments (Hoyng et al., 2015, Gene Ther 22, 767-780).

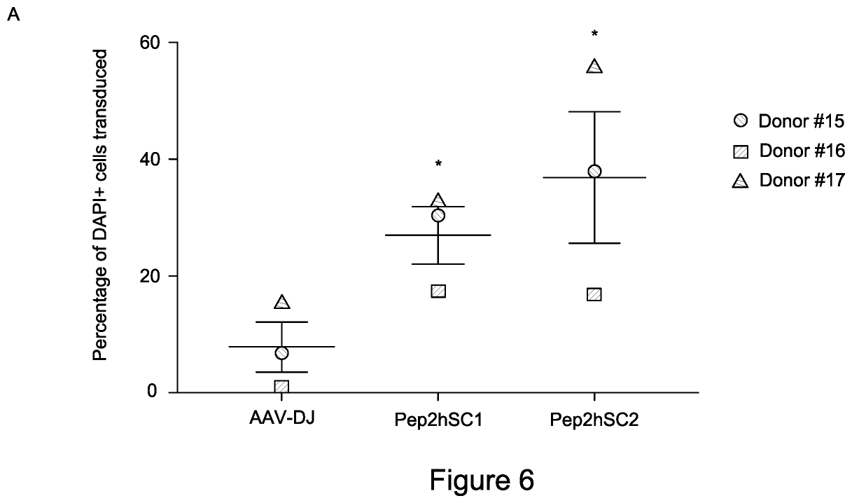

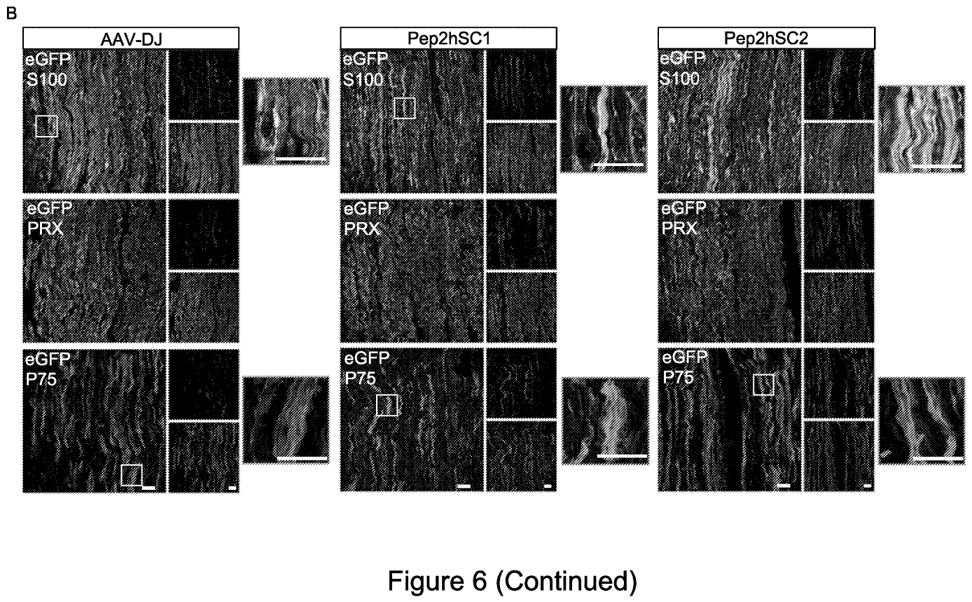

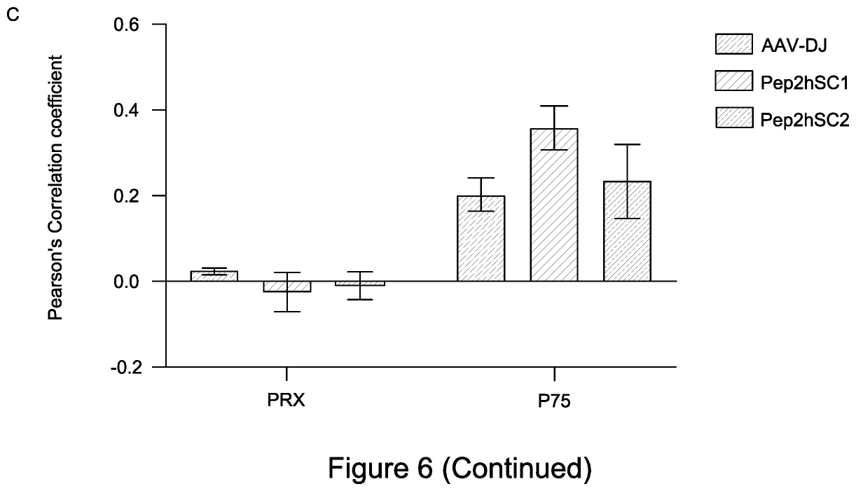

Therefore, the inventors subsequently examined the capacity of the Pep2hSC1 and Pep2hSC2 variants to transduce SCs in the context of human nerve segments. Sural nerve fascicles (~0.5 cm long) from 3 individual donors (n=1 segment per serotype and per donor) were injected with 1×10¹⁰ vg of either Pep2hSC1, Pep2hSC2, or AAV-DJ and assessed for expression of eGFP protein 7 and 14 days post-AAV administration (FIG. 6). Immunofluorescence of longitudinal sections of human sural nerve segments at 14 days post injection showed transduced cells with elongated morphology characteristic of SCs. Scanned sections showed a higher number of eGFP+ cells and a stronger eGFP intensity for Pep2hSC1 and Pep2hSC2 after 7 days compared to AAV-DJ. On day 14, immunofluorescent analysis showed that transduction with AAV-DJ results in a significantly lower percentage of eGFP+ cells than the novel AAV variants Pep2hSC1 and Pep2hSC2 (FIG. 6A).

Confocal imaging of the nerve segment revealed that the transduced cells had an elongated morphology with oval-shaped nuclei characteristic of hSCs. This was also confirmed with the co-labelling of eGFP and S100P (FIG. 6B). To further explore the tropism of Pep2hSC1 and Pep2hSC2, the inventors performed a series of double-labelling immunohistochemistry experiments and Pearson correlation coefficient analysis to determine the type of hSCs that were transduced. Interestingly, eGFP was rarely expressed by PRX-labelled myelinating SCs (mSCs). However, the transduced SCs were positive for p75 neurotrophin receptor (P75NTR) for two donors (FIG. 6C), which is a marker specific for non-myelinating SCs (nmSCs) or dedifferentiated SCs (Ma et al., 2018, J Neurosci 38, 9228-9239). Interestingly, for one donor, eGFP was co-expressed with PRX (data not shown), suggesting that the novel variants can potentially transduce mSCs.

In summary, these results demonstrate that Pep2hSC1 and Pep2hSC2 capsids can transduce nmSCs and mSCs in human nerve tissue after direct injection into nerve fascicles ex vivo.

### Example 6. Transduction Patterns Differ Between Pep2hSC1 and Pep2hSC2 in Injured Mouse Sciatic Nerve

Since the Pep2hSC1 and Pep2hSC2 capsid variants showed an improved transduction profile of rat and mouse SCs in vitro, the inventors next used a mouse peripheral nerve injury model as a surrogate to gauge the transduction efficiency in human nerves.

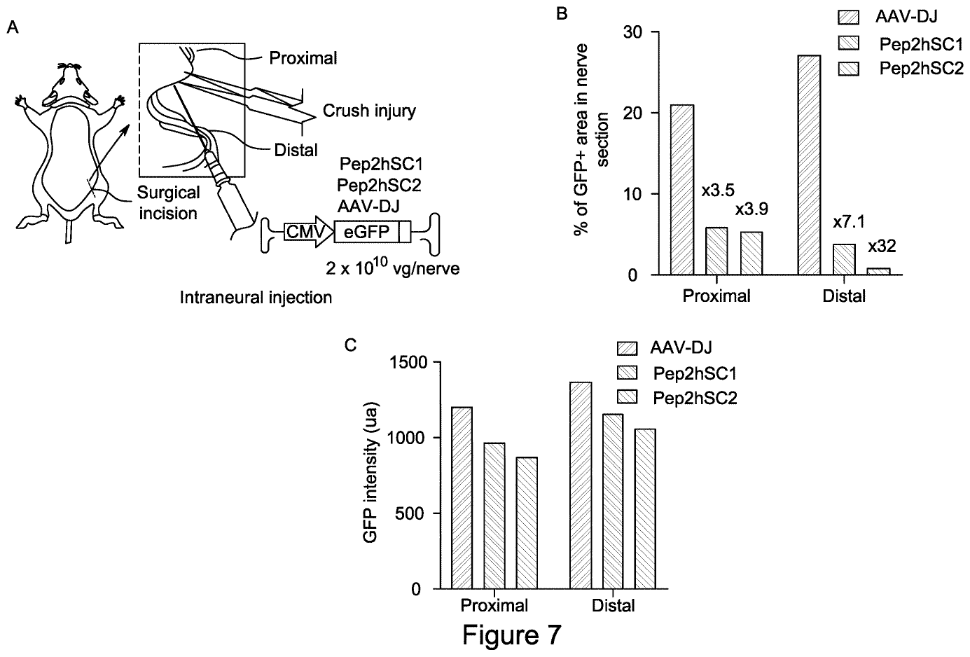

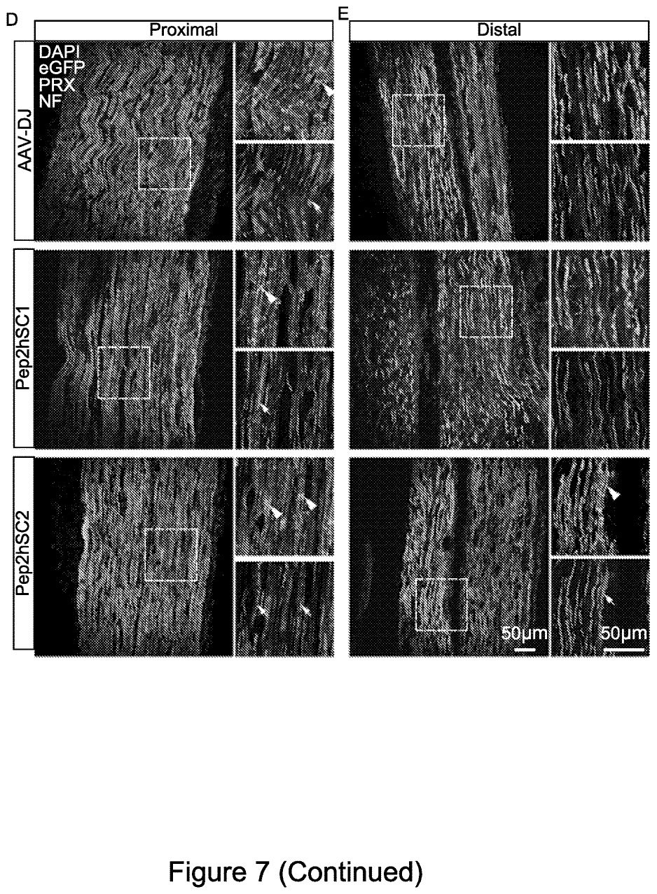

Equal amounts of Pep2hSC1, Pep2hSC2, and AAV-DJ vectors encoding single stranded AAV (ssAAV) CMV-eGFP-pA cassettes were injected into the sciatic nerves of adult mice at 2×10¹⁰ vg per animal following sciatic nerve crush (FIG. 7A). Four weeks later longitudinal sections of the sciatic nerve were examined for eGFP expression (FIGS. 7B and 7C) and were co-stained for both myelinating SC and axonal markers, PRX and neurofilament (NF), respectively (FIGS. 7D and 7E).

Following sciatic nerve crush, it was found that eGFP expression was more widespread following AAV-DJ injection compared to Pep2hSC1 and Pep2hSC2 with the biggest difference observed in the distal region (FIG. 7B). However, similar levels of eGFP fluorescence were observed for all three vectors (FIG. 7C).

For all the vectors tested, many eGFP cells within the area proximal to the crushed site (uninjured segment), displayed a morphology resembling mSCs with distinct appearance of cytoplasmic bands (FIG. 7D). Double labelling with the mSC marker PRX confirmed a close colocalization with eGFP (FIG. 7D). Furthermore, staining with an NF antibody confirmed eGFP ensheathed axons in a manner suggestive of myelin sheath formation (FIG. 7D). Notably, this cell type bias contrasts with the observation in human nerves, where the vectors showed a bias toward nmSCs.

Interestingly, within the lesioned area in the distal nerve, it was found that Pep2hSC1, Pep2hSC2 and AAV-DJ transduce distinct cell types (FIG. 7D). Pep2hSC1-driven transgene expression was detected exclusively in axons, positive for NF. Conversely, for Pep2hSC2, eGFP expression was observed in mSCs, while AAV-DJ targeted mostly nmSCs. eGFP was assessed in the spinal cord and both AAV-DJ and Pep2hSC1 transduced motor neurons, with Pep2hSC1 demonstrating superior performance, while Pep2hSC2 demonstrated very limited transduction efficiency.

These experiments indicate that Pep2hSC1 and Pep2hSC2 can transduce naive and injured murine SCs but exhibit unique tropism towards specific subtypes of SCs, particularly in the context of nerve injury.

### Example 7. Assessing Functional Off-Target Transduction of Primary Human Hepatocyte in Xenograft Liver Models In Vivo

The inventors also investigated the utility of Pep2hSC1 and Pep2hSC2 for systemic delivery by assessing the off-target potential of those vectors in vivo. Specifically, because the liver is a natural target for most natural and bioengineered AAVs and thus presents a major safety concern, the inventors used a humanized FRG (hFRG) model (Azuma et al., 2007, Nat Biotechnol 25, 903-910) to evaluate the Pep2hSC1 and Pep2hSC2 variants for their ability to transduce primary human hepatocytes in vivo.

Chimeric mice repopulated with primary human hepatocytes to a similar high replacement index (RI) were intravenously administered a dose of 2×10¹¹ vgs, representing ~1×10¹³ vg/kg of Pep2hSC1, Pep2hSC2, or the parental AAV2, encoding CMV-eGFP-pA reporter cassette. The relative performance in human hepatocytes was assessed by immunofluorescence and vector entry into human hepatocytes at the DNA level (data not shown) two weeks post-injection.

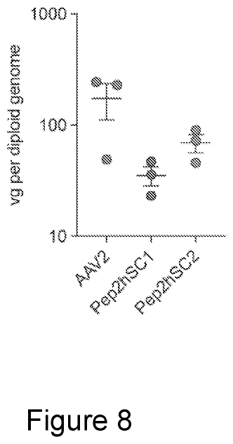

The Pep2hSC1 and Pep2hSC2 variants showed diminished entry into human hepatocytes when compared to parental AAV2, as evident by a respective 5-fold and 2.5-fold lower average VCN (vector copy number) per diploid human genome (FIG. 8). This may suggest a lower risk of acute liver toxicity arising from viral load in using Pep2hSC1 and Pep2hSC2.

TABLE 4
Capsid Sequences

| SEQ ID | Capsid | Capsid sequence |
|---|---|---|
| NO | name | (sequence corresponding to peptide insert in bold) |
| 1 | AAV2 | MAADGYLPDWLEDTLSEGIRQWWKLKPGPPPPKPAERHKDDSRGLVLPGYKYL |
| | capsid- | GPFNGLDKGEPVNEADAAALEHDKAYDRQLDSGDNPYLKYNHADAEFQERLKE |
| | VP1 | DTSFGGNLGRAVFQAKKRVLEPLGLVEEPVKTAPGKKRPVEHSPVEPDSSSGTG |
| | (protein) | KAGQQPARKRLNFGQTGDADSVPDPQPLGQPPAAPSGLGTNTMATGSGAPMA |
| | | DNNEGADGVGNSSGNWHCDSTWMGDRVITTSTRTWALPTYNNHLYKQISSQS |
| | | GASNDNHYFGYSTPWGYFDFNRFHCHFSPRDWQRLINNNWGFRPKRLNFKLFN |
| | | IQVKEVTQNDGTTTIANNLTSTVQVFTDSEYQLPYVLGSAHQGCLPPFPADVFMV |
| | | PQYGYLTLNNGSQAVGRSSFYCLEYFPSQMLRTGNNFTFSYTFEDVPFHSSYAHS |
| | | QSLDRLMNPLIDQYLYYLSRTNTPSGTTTQSRLQFSQAGASDIRDQSRNWLPGP |
| | | CYRQQRVSKTSADNNNSEYSWTGATKYHLNGRDSLVNPGPAMASHKDDEEKF |
| | | FPQSGVLIFGKQGSEKTNVDIEKVMITDEEEIRTTNPVATEQYGSVSTNLQRGNR |
| | | QAATADVNTQGVLPGMVWQDRDVYLQGPIWAKIPHTDGHFHPSPLMGGFGLK |
| | | HPPPQILIKNTPVPANPSTTFSAAKFASFITQYSTGQVSVEIEWELQKENSKRWN |
| | | PEIQYTSNYNKSVNVDFTVDTNGVYSEPRPIGTRYLTRNL |
| 2 | AAV9 | MAADGYLPDWLEDNLSEGIREWWALKPGAPQPKANQQHQDNARGLVLPGYKY |
| | capsid- | LGPGNGLDKGEPVNAADAAALEHDKAYDQQLKAGDNPYLKYNHADAEFQERLK |
| | VP1 | EDTSFGGNLGRAVFQAKKRLLEPLGLVEEAAKTAPGKKRPVEQSPQEPDSSAGI |
| | (protein) | GKSGAQPAKKRLNFGQTGDTESVPDPQPIGEPPAAPSGVGSLTMASGGGAPVA |
| | | DNNEGADGVGSSSGNWHCDSQWLGDRVITTSTRTWALPTYNNHLYKQISNST |
| | | SGGSSNDNAYFGYSTPWGYFDFNRFHCHFSPRDWQRLINNNWGFRPKRLNFKL |
| | | FNIQVKEVTDNNGVKTIANNLTSTVQVFTDSDYQLPYVLGSAHEGCLPPFPADVF |
| | | MIPQYGYLTLNDGSQAVGRSSFYCLEYFPSQMLRTGNNFQFSYEFENVPFHSSYA |
| | | HSQSLDRLMNPLIDQYLYYLSKTINGSGQNQQTLKFSVAGPSNMAVQGRNYIPG |
| | | PSYRQQRVSTTVTQNNNSEFAWPGASSWALNGRNSLMNPGPAMASHKEGEDR |
| | | FFPLSGSLIFGKQGTGRDNVDADKVMITNEEEIKTTNPVATESYGQVATNHQSA |
| | | QAQAQTGWVQNQGILPGMVWQDRDVYLQGPIWAKIPHTDGNFHPSPLMGGFG |
| | | MKHPPPQILIKNTPVPADPPTAFNKDKLNSFITQYSTGQVSVEIEWELQKENSKR |
| | | WNPEIQYTSNYYKSNNVEFAVNTEGVYSEPRPIGTRYLTRNL |
| 6 | Pep2hSC1 | MAADGYLPDWLEDTLSEGIRQWWKLKPGPPPPKPAERHKDDSRGLVLPGYKYL |
| | (protein) | GPFNGLDKGEPVNEADAAALEHDKAYDRQLDSGDNPYLKYNHADAEFQERLKE |
| | | DTSFGGNLGRAVFQAKKRVLEPLGLVEEPVKTAPGKKRPVEHSPVEPDSSSGTG |
| | | KAGQQPARKRLNFGQTGDADSVPDPQPLGQPPAAPSGLGTNTMATGSGAPMA |
| | | DNNEGADGVGNSSGNWHCDSTWMGDRVITTSTRTWALPTYNNHLYKQISSQS |
| | | GASNDNHYFGYSTPWGYFDFNRFHCHFSPRDWQRLINNNWGFRPKRLNFKLFN |
| | | IQVKEVTQNDGTTTIANNLTSTVQVFTDSEYQLPYVLGSAHQGCLPPFPADVFMV |
| | | PQYGYLTLNNGSQAVGRSSFYCLEYFPSQMLRTGNNFTFSYTFEDVPFHSSYAHS |
| | | QSLDRLMNPLIDQYLYYLSRTNTPSGTTTQSRLQFSQAGASDIRDQSRNWLPGP |
| | | CYRQQRVSKTSADNNNSEYSWTGATKYHLNGRDSLVNPGPAMASHKDDEEKF |
| | | FPQSGVLIFGKQGSEKTNVDIEKVMITDEEEIRTTNPVATEQYGSVSTNLQGQRK |
| | | RGDSWSEAAATADVNTQGVLPGMVWQDRDVYLQGPIWAKIPHTDGHFHPSPL |
| | | MGGFGLKHPPPQILIKNTPVPANPSTTFSAAKFASFITQYSTGQVSVEIEWELQKE |
| | | NSKRWNPEIQYTSNYNKSVNVDFTVDTNGVYSEPRPIGTRYLTRNL |
| 7 | Pep2hSC2 | MAADGYLPDWLEDTLSEGIRQWWKLKPGPPPPKPAERHKDDSRGLVLPGYKYL |
| | (protein) | GPFNGLDKGEPVNEADAAALEHDKAYDRQLDSGDNPYLKYNHADAEFQERLKE |
| | | DTSFGGNLGRAVFQAKKRVLEPLGLVEEPVKTAPGKKRPVEHSPVEPDSSSGTG |
| | | KAGQQPARKRLNFGQTGDADSVPDPQPLGQPPAAPSGLGTNTMATGSGAPMA |
| | | DNNEGADGVGNSSGNWHCDSTWMGDRVITTSTRTWALPTYNNHLYKQISSQS |
| | | GASNDNHYFGYSTPWGYFDFNRFHCHFSPRDWQRLINNNWGFRPKRLNFKLFN |
| | | IQVKEVTQNDGTTTIANNLTSTVQVFTDSEYQLPYVLGSAHQGCLPPFPADVFMV |
| | | PQYGYLTLNNGSQAVGRSSFYCLEYFPSQMLRTGNNFTFSYTFEDVPFHSSYAHS |
| | | QSLDRLMNPLIDQYLYYLSRTNTPSGTTTQSRLQFSQAGASDIRDQSRNWLPGP |
| | | CYRQQRVSKTSADNNNSEYSWTGATKYHLNGRDSLVNPGPAMASHKDDEEKF |
| | | FPQSGVLIFGKQGSEKTNVDIEKVMITDEEEIRTTNPVATEQYGSVSTNLQGQRR |
| | | KDVDFDKAAATADVNTQGVLPGMVWQDRDVYLQGPIWAKIPHTDGHFHPSPL |
| | | MGGFGLKHPPPQILIKNTPVPANPSTTFSAAKFASFITQYSTGQVSVEIEWELQKE |
| | | NSKRWNPEIQYTSNYNKSVNVDFTVDTNGVYSEPRPIGTRYLTRNL |
| 14 | Pep9hSC1 | MAADGYLPDWLEDNLSEGIREWWALKPGAPQPKANQQHQDNARGLVLPGYKY |
| | (protein) | LGPGNGLDKGEPVNAADAAALEHDKAYDQQLKAGDNPYLKYNHADAEFQERLK |
| | | EDTSFGGNLGRAVFQAKKRLLEPLGLVEEAAKTAPGKKRPVEQSPQEPDSSAGI |
| | | GKSGAQPAKKRLNFGQTGDTESVPDPQPIGEPPAAPSGVGSLTMASGGGAPVA |
| | | DNNEGADGVGSSSGNWHCDSQWLGDRVITTSTRTWALPTYNNHLYKQISNST |
| | | SGGSSNDNAYFGYSTPWGYFDFNRFHCHFSPRDWQRLINNNWGFRPKRLNFKL |
| | | FNIQVKEVTDNNGVKTIANNLTSTVQVFTDSDYQLPYVLGSAHEGCLPPFPADVF |
| | | MIPQYGYLTLNDGSQAVGRSSFYCLEYFPSQMLRTGNNFQFSYEFENVPFHSSYA |
| | | HSQSLDRLMNPLIDQYLYYLSKTINGSGQNQQTLKFSVAGPSNMAVQGRNYIPG |
| | | PSYRQQRVSTTVTQNNNSEFAWPGASSWALNGRNSLMNPGPAMASHKEGEDR |
| | | FFPLSGSLIFGKQGTGRDNVDADKVMITNEEEIKTTNPVATESYGQVATNHQGQ |
| | | SARGDVRVQAAQTGWVQNQGILPGMVWQDRDVYLQGPIWAKIPHTDGNFHPS |
| | | PLMGGFGMKHPPPQILIKNTPVPADPPTAFNKDKLNSFITQYSTGQVSVEIEWEL |
| | | QKENSKRWNPEIQYTSNYYKSNNVEFAVNTEGVYSEPRPIGTRYLTRNL |
| 15 | Pep9hSC2 | MAADGYLPDWLEDNLSEGIREWWALKPGAPQPKANQQHQDNARGLVLPGYKY |
| | (protein) | LGPGNGLDKGEPVNAADAAALEHDKAYDQQLKAGDNPYLKYNHADAEFQERLK |
| | | EDTSFGGNLGRAVFQAKKRLLEPLGLVEEAAKTAPGKKRPVEQSPQEPDSSAGI |
| | | GKSGAQPAKKRLNFGQTGDTESVPDPQPIGEPPAAPSGVGSLTMASGGGAPVA |
| | | DNNEGADGVGSSSGNWHCDSQWLGDRVITTSTRTWALPTYNNHLYKQISNST |
| | | SGGSSNDNAYFGYSTPWGYFDFNRFHCHFSPRDWQRLINNNWGFRPKRLNFKL |
| | | FNIQVKEVTDNNGVKTIANNLTSTVQVFTDSDYQLPYVLGSAHEGCLPPFPADVF |
| | | MIPQYGYLTLNDGSQAVGRSSFYCLEYFPSQMLRTGNNFQFSYEFENVPFHSSYA |
| | | HSQSLDRLMNPLIDQYLYYLSKTINGSGQNQQTLKFSVAGPSNMAVQGRNYIPG |
| | | PSYRQQRVSTTVTQNNNSEFAWPGASSWALNGRNSLMNPGPAMASHKEGEDR |
| | | FFPLSGSLIFGKQGTGRDNVDADKVMITNEEEIKTTNPVATESYGQVATNHQGQ |
| | | RGRGDVAGEAAQTGWVQNQGILPGMVWQDRDVYLQGPIWAKIPHTDGNFHPS |
| | | PLMGGFGMKHPPPQILIKNTPVPADPPTAFNKDKLNSFITQYSTGQVSVEIEWEL |
| | | QKENSKRWNPEIQYTSNYYKSNNVEFAVNTEGVYSEPRPIGTRYLTRNL |
| 16 | Pep9hSC5 | MAADGYLPDWLEDNLSEGIREWWALKPGAPQPKANQQHQDNARGLVLPGYKY |
| | (protein) | LGPGNGLDKGEPVNAADAAALEHDKAYDQQLKAGDNPYLKYNHADAEFQERLK |
| | | EDTSFGGNLGRAVFQAKKRLLEPLGLVEEAAKTAPGKKRPVEQSPQEPDSSAGI |
| | | GKSGAQPAKKRLNFGQTGDTESVPDPQPIGEPPAAPSGVGSLTMASGGGAPVA |
| | | DNNEGADGVGSSSGNWHCDSQWLGDRVITTSTRTWALPTYNNHLYKQISNST |
| | | SGGSSNDNAYFGYSTPWGYFDFNRFHCHFSPRDWQRLINNNWGFRPKRLNFKL |
| | | FNIQVKEVTDNNGVKTIANNLTSTVQVFTDSDYQLPYVLGSAHEGCLPPFPADVF |
| | | MIPQYGYLTLNDGSQAVGRSSFYCLEYFPSQMLRTGNNFQFSYEFENVPFHSSYA |
| | | HSQSLDRLMNPLIDQYLYYLSKTINGSGQNQQTLKFSVAGPSNMAVQGRNYIPG |
| | | PSYRQQRVSTTVTQNNNSEFAWPGASSWALNGRNSLMNPGPAMASHKEGEDR |
| | | FFPLSGSLIFGKQGTGRDNVDADKVMITNEEEIKTTNPVATESYGQVATNHQGQ |
| | | SRADRPPPQAAQTGWVQNQGILPGMVWQDRDVYLQGPIWAKIPHTDGNFHPS |
| | | PLMGGFGMKHPPPQILIKNTPVPADPPTAFNKDKLNSFITQYSTGQVSVEIEWEL |
| | | QKENSKRWNPEIQYTSNYYKSNNVEFAVNTEGVYSEPRPIGTRYLTRNL |
| 17 | Pep9hSC9 | MAADGYLPDWLEDNLSEGIREWWALKPGAPQPKANQQHQDNARGLVLPGYKY |
| | (protein) | LGPGNGLDKGEPVNAADAAALEHDKAYDQQLKAGDNPYLKYNHADAEFQERLK |
| | | EDTSFGGNLGRAVFQAKKRLLEPLGLVEEAAKTAPGKKRPVEQSPQEPDSSAGI |
| | | GKSGAQPAKKRLNFGQTGDTESVPDPQPIGEPPAAPSGVGSLTMASGGGAPVA |
| | | DNNEGADGVGSSSGNWHCDSQWLGDRVITTSTRTWALPTYNNHLYKQISNST |
| | | SGGSSNDNAYFGYSTPWGYFDFNRFHCHFSPRDWQRLINNNWGFRPKRLNFKL |
| | | FNIQVKEVTDNNGVKTIANNLTSTVQVFTDSDYQLPYVLGSAHEGCLPPFPADVF |
| | | MIPQYGYLTLNDGSQAVGRSSFYCLEYFPSQMLRTGNNFQFSYEFENVPFHSSYA |
| | | HSQSLDRLMNPLIDQYLYYLSKTINGSGQNQQTLKFSVAGPSNMAVQGRNYIPG |
| | | PSYRQQRVSTTVTQNNNSEFAWPGASSWALNGRNSLMNPGPAMASHKEGEDR |
| | | FFPLSGSLIFGKQGTGRDNVDADKVMITNEEEIKTTNPVATESYGQVATNHQGQ |
| | | SARGDMLGKAAQTGWVQNQGILPGMVWQDRDVYLQGPIWAKIPHTDGNFHPS |
| | | PLMGGFGMKHPPPQILIKNTPVPADPPTAFNKDKLNSFITQYSTGQVSVEIEWEL |
| | | QKENSKRWNPEIQYTSNYYKSNNVEFAVNTEGVYSEPRPIGTRYLTRNL |
| 18 | Shuffle32 | MAADGYLPDWLEDNLSEGIREWWDLKPGAPKPKANQQKQDDGRGLVLPGYKY |
| | (protein) | LGPFNGLDKGEPVNEADAAALEHDKAYDQQLKAGDNPYLRYNHADAEFQERLQ |
| | | EDTSFGGNLGRAVFQAKKRLLEPLGLVEEAAKTAPAKKRPVEPSPQRSPDSSTGI |
| | | GKKGQQPARKRLNFGQTGDSESVPDPQPLGEPPATPAAVGPTTMASGGGAPMA |
| | | DNNEGADGVGNASGNWHCDSTWLGDRVITTSTRTWALPTYNNHLYKQISSQS |
| | | GASNDNHYFGYSTPWGYFDFNRFHCHFSPRDWQRLINNNWGFRPKRLNFKLFN |
| | | IQVKEVTTNDGVTTIANNLTSTVQVFSDSEYQLPYVLGSAHQGCLPPFPADVFMI |
| | | PQYGYLTLNNGSQAVGRSSFYCLEYFPSQMLRTGNNFTFSYTFEDVPFHSSYAHS |
| | | QSLDRLMNPLIDQYLYYLSRTNTPSGTTTQSRLQFSQAGASDIRDQSRNWLPGP |
| | | CYRQQRVSKTSADNNNSEYSWTGATKYHLNGRDSLVNPGPAMASHKDDEEKF |
| | | FPQSGVLIFGKQGSEKTNVDIEKVMITDEEEIRTTNPVATEQYGSVSTNLQRGNR |
| | | QAATADVNTQGVLPGMVWQDRDVYLQGPIWAKIPHTDGHFHPSPLMGGFGLK |
| | | HPPPQILIKNTPVPANPSTTFSAAKFASFITQYSTGQVSVEIEWELQKENSKRWN |
| | | PEIQYTSNYYKSTNVDFAVDSQGVYSEPRPIGTRYLTRNL |
| 19 | Shuffle35 | MAADGYLPDWLEDNLSEGIRQWWKLKPGPPPPKPAERHKDDSRGLVLPGYKYL |
| | (protein) | GPFNGLDKGEPVNEADAAALEHDKAYDQQLKAGDNPYLRYNHADAEFQERLQE |
| | | DTSFGGNLGRAVFQAKKRVLEPLGLVEEGAKTAPGKKRPVEQSPQEPDSSAGIG |
| | | KSGAQPAKKRLNFGQTGDSESVPDPQPLGEPPATPAAVGPTTMASGGGAPMAD |
| | | NNEGADGVGNASGNWHCDSTWLGDRVITTSTRTWALPTYNNHLYKQISSAST |
| | | GASNDNHYFGYSTPWGYFDFNRFHCHFSPRDWQRLINNNGGFRPKRLNFKLFNI |
| | | QVKEVTTNDGVTTIANNLTSTVQVFSDSEYQLPYVLGSAHQGCLPPFPADVFMIP |
| | | QYGYLTLNNGSQAVGRSSFYCLEYFPSQMLRTGNNFTFSYTFEDVPFHSSYAHS |
| | | QSLDRLMNPLIDQYLYYLSRTNTPSGTTTQSRLQFSQAGASDIRDQSRNWLPGP |
| | | CYRQQRVSKTSADNNNSEYSWTGATKYHLNGRDSLVNPGPAMASHKDDEEKF |
| | | FPQSGVLIFGKQGSEKTNVDIEKVMITDEEEIRTTNPVATEQYGSVSTNLQRGNR |
| | | QAATADVNTQGVLPGMVWQDRDVYLQGPIWAKIPHTDGHFHPSPLMGGFGLK |
| | | HPPPQIMIKNTPVPANPSTTFSAAKFASFITQYSTGQVSVEIEWELQKENSKRWN |
| | | PEIQYTSNYYKSTSVDFAVNTEGVYSEPRPIGTRYLTRNL |
| 20 | Pep2hSC1 | ATGGCTGCCGATGGTTATCTTCCAGATTGGCTCGAGGACACTCTCTCTGAAGG |
| | (DNA) | CATTCGCCAGTGGTGGAAGTTGAAACCTGGACCACCGCCACCCAAACCCGCA |
| | | GAGCGGCATAAGGACGACAGCCGGGGTCTTGTGCTTCCTGGTTACAAGTACC |
| | | TCGGACCCTTCAACGGACTCGACAAGGGGGAGCCCGTCAACGAGGCGGACG |
| | | CAGCGGCCCTCGAGCACGACAAGGCCTACGACCGGCAGCTCGACAGCGGTG |
| | | ACAACCCGTACCTCAAGTACAACCACGCCGACGCCGAGTTTCAGGAGCGTCT |
| | | GAAAGAAGATACGTCTTTTGGGGGCAACCTCGGGCGAGCAGTCTTCCAGGCC |
| | | AAGAAGAGGGTTCTCGAACCTCTTGGTCTGGTTGAGGAACCTGTTAAGACGG |
| | | CTCCTGGAAAGAAGCGTCCGGTAGAGCACTCTCCAGTGGAGCCAGACTCCTC |
| | | CTCGGGCACCGGCAAGGCGGGCCAGCAGCCCGCTAGAAAGAGACTCAATTTT |
| | | GGTCAGACTGGCGACGCAGACTCAGTCCCCGACCCTCAACCTCTCGGACAGC |
| | | CTCCAGCAGCCCCCTCTGGTTTGGGAACTAATACAATGGCTACAGGCAGTGG |
| | | CGCACCAATGGCAGACAATAACGAAGGCGCCGACGGAGTGGGTAATTCCTCG |
| | | GGAAATTGGCATTGCGATTCCACATGGATGGGCGACAGAGTCATCACCACCA |
| | | GCACCCGAACCTGGGCCCTGCCCACCTACAACAACCACCTCTACAAGCAAATC |
| | | AGTAGCCAATCAGGAGCCAGCAACGACAACCACTACTTCGGCTACAGCACCC |
| | | CCTGGGGGTATTTTGACTTCAACAGATTCCACTGCCACTTCTCACCACGTGAC |
| | | TGGCAGCGACTCATCAACAACAACTGGGGATTCCGGCCCAAGAGACTCAACT |
| | | TCAAGCTCTTCAACATCCAAGTCAAGGAGGTCACGCAGAATGATGGCACGAC |
| | | GACCATCGCCAATAACCTTACCAGCACGGTTCAAGTGTTTACGGACTCGGAGT |
| | | ACCAGCTCCCGTACGTCCTCGGCTCTGCGCACCAGGGCTGCCTCCCTCCGTT |
| | | CCCGGCGGACGTCTTCATGGTGCCGCAGTACGGCTACCTGACGCTGAACAAC |
| | | GGCAGCCAGGCCGTGGGACGCTCATCCTTTTACTGCCTGGAATACTTCCCTTC |
| | | GCAGATGCTGAGAACGGGCAACAACTTTACCTTCAGCTACACCTTTGAGGAC |
| | | GTGCCTTTCCACAGCAGCTACGCTCACAGCCAGAGCCTGGACCGGCTGATGA |
| | | ATCCTCTCATCGACCAGTACCTGTACTACCTGAGCAGAACTAACACTCCATCC |
| | | GGAACCACCACGCAATCAAGGTTGCAGTTTAGCCAGGCCGGAGCGAGTGACA |
| | | TTCGGGACCAGTCTAGAAACTGGCTACCTGGACCCTGTTACCGGCAGCAGCG |
| | | CGTTTCAAAGACATCTGCGGACAACAACAACAGCGAATACTCGTGGACTGGT |
| | | GCTACCAAATATCATCTCAATGGCCGTGACTCTCTGGTGAATCCTGGCCCGGC |
| | | TATGGCCTCACACAAAGACGACGAAGAAAAGTTCTTTCCCCAGAGCGGTGTC |
| | | CTCATTTTTGGCAAACAAGGCTCAGAGAAAACAAACGTGGATATTGAAAAGGT |
| | | CATGATTACCGACGAAGAGGAAATTAGGACCACTAACCCTGTGGCCACGGAG |
| | | CAGTATGGGTCTGTGTCTACCAACCTCCAGGGCCAGAGGAAGCGGGGTGATT |
| | | CGTGGTCTGAGGCGGCCGCGACCGCAGATGTCAACACACAGGGAGTTTTACC |
| | | TGGCATGGTGTGGCAAGACAGAGACGTGTACCTGCAGGGTCCCATTTGGGCC |
| | | AAGATTCCTCACACGGATGGACACTTTCACCCGTCTCCGCTGATGGGCGGCTT |
| | | TGGACTGAAACACCCGCCTCCTCAGATCCTCATCAAAAACACGCCTGTACCTG |
| | | CGAATCCTTCGACCACCTTCAGCGCTGCAAAGTTTGCTTCATTCATCACCCAG |
| | | TACTCCACTGGACAGGTCAGCGTGGAGATTGAGTGGGAGCTGCAGAAGGAAA |
| | | ACAGCAAGCGCTGGAATCCCGAGATCCAGTACACATCCAACTACAACAAATCT |
| | | GTTAATGTGGACTTTACTGTGGACACTAATGGAGTGTATAGTGAGCCTCGCCC |
| | | CATTGGCACCCGTTACCTCACCCGTAATCTGTAA |
| 21 | Pep2hSC2 | ATGGCTGCCGATGGTTATCTTCCAGATTGGCTCGAGGACACTCTCTCTGAAGG |
| | (DNA) | CATTCGCCAGTGGTGGAAGTTGAAACCTGGACCACCGCCACCCAAACCCGCA |
| | | GAGCGGCATAAGGACGACAGCCGGGGTCTTGTGCTTCCTGGTTACAAGTACC |
| | | TCGGACCCTTCAACGGACTCGACAAGGGGGAGCCCGTCAACGAGGCGGACG |
| | | CAGCGGCCCTCGAGCACGACAAGGCCTACGACCGGCAGCTCGACAGCGGTG |
| | | ACAACCCGTACCTCAAGTACAACCACGCCGACGCCGAGTTTCAGGAGCGTCT |
| | | GAAAGAAGATACGTCTTTTGGGGGCAACCTCGGGCGAGCAGTCTTCCAGGCC |
| | | AAGAAGAGGGTTCTCGAACCTCTTGGTCTGGTTGAGGAACCTGTTAAGACGG |
| | | CTCCTGGAAAGAAGCGTCCGGTAGAGCACTCTCCAGTGGAGCCAGACTCCTC |
| | | CTCGGGCACCGGCAAGGCGGGCCAGCAGCCCGCTAGAAAGAGACTCAATTTT |
| | | GGTCAGACTGGCGACGCAGACTCAGTCCCCGACCCTCAACCTCTCGGACAGC |
| | | CTCCAGCAGCCCCCTCTGGTTTGGGAACTAATACAATGGCTACAGGCAGTGG |
| | | CGCACCAATGGCAGACAATAACGAAGGCGCCGACGGAGTGGGTAATTCCTCG |
| | | GGAAATTGGCATTGCGATTCCACATGGATGGGCGACAGAGTCATCACCACCA |
| | | GCACCCGAACCTGGGCCCTGCCCACCTACAACAACCACCTCTACAAGCAAATC |
| | | AGTAGCCAATCAGGAGCCAGCAACGACAACCACTACTTCGGCTACAGCACCC |
| | | CCTGGGGGTATTTTGACTTCAACAGATTCCACTGCCACTTCTCACCACGTGAC |
| | | TGGCAGCGACTCATCAACAACAACTGGGGATTCCGGCCCAAGAGACTCAACT |
| | | TCAAGCTCTTCAACATCCAAGTCAAGGAGGTCACGCAGAATGATGGCACGAC |
| | | GACCATCGCCAATAACCTTACCAGCACGGTTCAAGTGTTTACGGACTCGGAGT |
| | | ACCAGCTCCCGTACGTCCTCGGCTCTGCGCACCAGGGCTGCCTCCCTCCGTT |
| | | CCCGGCGGACGTCTTCATGGTGCCGCAGTACGGCTACCTGACGCTGAACAAC |
| | | GGCAGCCAGGCCGTGGGACGCTCATCCTTTTACTGCCTGGAATACTTCCCTTC |
| | | GCAGATGCTGAGAACGGGCAACAACTTTACCTTCAGCTACACCTTTGAGGAC |
| | | GTGCCTTTCCACAGCAGCTACGCTCACAGCCAGAGCCTGGACCGGCTGATGA |
| | | ATCCTCTCATCGACCAGTACCTGTACTACCTGAGCAGAACTAACACTCCATCC |
| | | GGAACCACCACGCAATCAAGGTTGCAGTTTAGCCAGGCCGGAGCGAGTGACA |
| | | TTCGGGACCAGTCTAGAAACTGGCTACCTGGACCCTGTTACCGGCAGCAGCG |
| | | CGTTTCAAAGACATCTGCGGACAACAACAACAGCGAATACTCGTGGACTGGT |
| | | GCTACCAAATATCATCTCAATGGCCGTGACTCTCTGGTGAATCCTGGCCCGGC |
| | | TATGGCCTCACACAAAGACGACGAAGAAAAGTTCTTTCCCCAGAGCGGTGTC |
| | | CTCATTTTTGGCAAACAAGGCTCAGAGAAAACAAACGTGGATATTGAAAAGGT |
| | | CATGATTACCGACGAAGAGGAAATTAGGACCACTAACCCTGTGGCCACGGAG |
| | | CAGTATGGGTCTGTGTCTACCAACCTCCAGGGCCAGAGGAGGAAGGATGTTG |
| | | ATTTTGATAAGGCGGCCGCGACCGCAGATGTCAACACACAGGGAGTTTTACC |
| | | TGGCATGGTGTGGCAAGACAGAGACGTGTACCTGCAGGGTCCCATTTGGGCC |
| | | AAGATTCCTCACACGGATGGACACTTTCACCCGTCTCCGCTGATGGGGGGCTT |
| | | TGGACTGAAACACCCGCCTCCTCAGATCCTCATCAAAAACACGCCTGTACCTG |
| | | CGAATCCTTCGACCACCTTCAGCGCTGCAAAGTTTGCTTCATTCATCACCCAG |
| | | TACTCCACTGGACAGGTCAGCGTGGAGATTGAGTGGGAGCTGCAGAAGGAAA |
| | | ACAGCAAGCGCTGGAATCCCGAGATCCAGTACACATCCAACTACAACAAATCT |
| | | GTTAATGTGGACTTTACTGTGGACACTAATGGAGTGTATAGTGAGCCTCGCCC |
| | | CATTGGCACCCGTTACCTCACCCGTAATCTGTAA |
| 22 | Pep9hSC1 | ATGGCTGCCGATGGTTATCTTCCAGATTGGCTCGAGGACAACCTCTCTGAAGG |
| | (DNA) | CATTCGCGAGTGGTGGGCTTTGAAACCTGGAGCCCCGCAACCCAAAGCCAAC |
| | | CAGCAACATCAGGACAACGCTCGGGGTCTTGTGCTTCCTGGTTACAAGTACCT |
| | | CGGACCCGGCAACGGACTCGACAAGGGGGAGCCCGTCAACGCGGCGGACGC |
| | | AGCGGCCCTCGAGCACGACAAGGCCTACGACCAGCAGCTCAAGGCGGGTGA |
| | | CAACCCGTACCTCAAGTACAACCACGCCGACGCCGAGTTTCAGGAGCGTCTG |
| | | AAAGAAGATACGTCTTTTGGGGGCAACCTCGGGCGAGCAGTCTTCCAGGCCA |
| | | AGAAGAGGCTTCTCGAACCTCTTGGTCTGGTTGAGGAAGCGGCTAAGACGGC |
| | | TCCTGGAAAGAAGCGTCCGGTAGAGCAGTCTCCACAGGAGCCAGACTCCTCC |
| | | GCGGGCATCGGCAAGTCGGGCGCACAGCCCGCTAAAAAGAGACTCAATTTTG |
| | | GTCAGACTGGCGACACAGAGTCAGTCCCCGACCCTCAACCTATCGGAGAACC |
| | | TCCAGCAGCCCCCTCTGGTGTGGGATCTCTTACAATGGCTTCAGGCGGTGGC |
| | | GCACCAGTCGCAGACAATAACGAAGGCGCCGACGGAGTGGGTAGTTCCTCG |
| | | GGAAATTGGCATTGCGATTCCCAATGGCTGGGCGACAGAGTCATCACCACCA |
| | | GCACCCGAACCTGGGCCCTGCCCACCTACAACAACCACCTCTACAAGCAAATC |
| | | TCCAACAGTACATCAGGAGGAAGCAGCAACGACAACGCCTACTTCGGCTACA |
| | | GCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGAC |
| | | TCAACTTCAAGCTCTTCAACATCCAAGTCAAGGAGGTCACGGACAATAACGGC |
| | | GTCAAGACCATCGCCAATAACCTTACCAGCACGGTTCAAGTGTTTACGGACTC |
| | | GGACTACCAGCTCCCGTACGTCCTCGGCTCTGCGCACGAGGGCTGCCTCCCT |
| | | CCGTTCCCGGCGGACGTCTTCATGATTCCGCAGTACGGCTACCTGACGCTCA |
| | | ACGATGGCAGCCAGGCCGTGGGACGCTCATCCTTTTACTGCCTGGAATACTT |
| | | CCCTTCGCAGATGCTGAGAACGGGCAACAACTTTCAGTTCAGCTACGAGTITG |
| | | AGAACGTGCCTTTCCACAGCAGCTACGCTCACAGCCAGAGCCTGGACCGGCT |
| | | GATGAATCCTCTCATCGACCAGTACCTGTACTACCTGAGCAAGACTATTAATG |
| | | GATCTGGACAGAATCAACAGACCTTGAAATTTAGCGTGGCCGGACCTAGTAAC |
| | | ATGGCCGTTCAGGGAAGAAACTACATACCTGGACCCTCATACCGGCAGCAGC |
| | | GCGTTTCAACGACAGTGACACAAAACAACAACAGCGAATTTGCCTGGCCTGGT |
| | | GCTTCATCTTGGGCTCTCAATGGCCGTAATTCTCTGATGAATCCTGGCCCGGC |
| | | TATGGCCTCACACAAAGAAGGAGAAGACCGTTTCTTTCCCCTGAGCGGTTCTC |
| | | TCATTTTTGGCAAACAAGGCACAGGAAGAGACAACGTGGATGCGGACAAGGT |
| | | CATGATTACCAACGAAGAGGAAATTAAAACCACTAACCCTGTGGCCACGGAGT |
| | | CCTATGGGCAGGTGGCAACCAACCACCAGGGCCAGAGTGCGAGGGGCGACG |
| | | TGCGCGTCCAGGCGGCCCAAACCGGATGGGTCCAAAACCAGGGAATCTTACC |
| | | TGGCATGGTGTGGCAAGACAGAGACGTGTACCTGCAGGGTCCCATTTGGGCC |
| | | AAGATTCCTCACACGGATGGAAACTTTCACCCGTCTCCGCTGATGGGCGGCTT |
| | | TGGAATGAAACACCCGCCTCCTCAGATCCTCATCAAAAACACGCCTGTACCTG |
| | | CGGATCCTCCGACCGCCTTCAACAAGGACAAGCTGAACTCATTCATCACCCAG |
| | | TACTCCACTGGACAGGTCAGCGTGGAGATTGAGTGGGAGCTGCAGAAGGAAA |
| | | ACAGCAAGCGCTGGAATCCCGAGATCCAGTACACATCCAACTACTACAAATCT |
| | | AACAATGTGGAATTTGCTGTGAATACTGAAGGAGTGTATAGTGAGCCTCGCCC |
| | | CATTGGCACCCGTTACCTCACCCGTAATCTGTAA |
| 23 | Pep9hSC2 | ATGGCTGCCGATGGTTATCTTCCAGATTGGCTCGAGGACAACCTCTCTGAAGG |
| | (DNA) | CATTCGCGAGTGGTGGGCTTTGAAACCTGGAGCCCCGCAACCCAAAGCCAAC |
| | | CAGCAACATCAGGACAACGCTCGGGGTCTTGTGCTTCCTGGTTACAAGTACCT |
| | | CGGACCCGGCAACGGACTCGACAAGGGGGAGCCCGTCAACGCGGCGGACGC |
| | | AGCGGCCCTCGAGCACGACAAGGCCTACGACCAGCAGCTCAAGGCGGGTGA |
| | | CAACCCGTACCTCAAGTACAACCACGCCGACGCCGAGTTTCAGGAGCGTCTG |
| | | AAAGAAGATACGTCTTTTGGGGGCAACCTCGGGCGAGCAGTCTTCCAGGCCA |
| | | AGAAGAGGCTTCTCGAACCTCTTGGTCTGGTTGAGGAAGCGGCTAAGACGGC |
| | | TCCTGGAAAGAAGCGTCCGGTAGAGCAGTCTCCACAGGAGCCAGACTCCTCC |
| | | GCGGGCATCGGCAAGTCGGGCGCACAGCCCGCTAAAAAGAGACTCAATTTTG |
| | | GTCAGACTGGCGACACAGAGTCAGTCCCCGACCCTCAACCTATCGGAGAACC |
| | | TCCAGCAGCCCCCTCTGGTGTGGGATCTCTTACAATGGCTTCAGGCGGTGGC |
| | | GCACCAGTCGCAGACAATAACGAAGGCGCCGACGGAGTGGGTAGTTCCTCG |
| | | GGAAATTGGCATTGCGATTCCCAATGGCTGGGCGACAGAGTCATCACCACCA |
| | | GCACCCGAACCTGGGCCCTGCCCACCTACAACAACCACCTCTACAAGCAAATC |
| | | TCCAACAGTACATCAGGAGGAAGCAGCAACGACAACGCCTACTTCGGCTACA |
| | | GCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGAC |
| | | TCAACTTCAAGCTCTTCAACATCCAAGTCAAGGAGGTCACGGACAATAACGGC |
| | | GTCAAGACCATCGCCAATAACCTTACCAGCACGGTTCAAGTGTTTACGGACTC |
| | | GGACTACCAGCTCCCGTACGTCCTCGGCTCTGCGCACGAGGGCTGCCTCCCT |
| | | CCGTTCCCGGCGGACGTCTTCATGATTCCGCAGTACGGCTACCTGACGCTCA |
| | | ACGATGGCAGCCAGGCCGTGGGACGCTCATCCTTTTACTGCCTGGAATACTT |
| | | CCCTTCGCAGATGCTGAGAACGGGCAACAACTTTCAGTTCAGCTACGAGTTTG |
| | | AGAACGTGCCTTTCCACAGCAGCTACGCTCACAGCCAGAGCCTGGACCGGCT |
| | | GATGAATCCTCTCATCGACCAGTACCTGTACTACCTGAGCAAGACTATTAATG |
| | | GATCTGGACAGAATCAACAGACCTTGAAATTTAGCGTGGCCGGACCTAGTAAC |
| | | ATGGCCGTTCAGGGAAGAAACTACATACCTGGACCCTCATACCGGCAGCAGC |
| | | GCGTTTCAACGACAGTGACACAAAACAACAACAGCGAATTTGCCTGGCCTGGT |
| | | GCTTCATCTTGGGCTCTCAATGGCCGTAATTCTCTGATGAATCCTGGCCCGGC |
| | | TATGGCCTCACACAAAGAAGGAGAAGACCGTTTCTTTCCCCTGAGCGGTTCTC |
| | | TCATTTTTGGCAAACAAGGCACAGGAAGAGACAACGTGGATGCGGACAAGGT |
| | | CATGATTACCAACGAAGAGGAAATTAAAACCACTAACCCTGTGGCCACGGAGT |
| | | CCTATGGGCAGGTGGCAACCAACCACCAGGGCCAGAGGGGTCGGGGTGATG |
| | | TGGCTGGGGAGGCGGCCCAAACCGGATGGGTCCAAAACCAGGGAATCTTAC |
| | | CTGGCATGGTGTGGCAAGACAGAGACGTGTACCTGCAGGGTCCCATTTGGGC |
| | | CAAGATTCCTCACACGGATGGAAACTTTCACCCGTCTCCGCTGATGGGCGGCT |
| | | TTGGAATGAAACACCCGCCTCCTCAGATCCTCATCAAAAACACGCCTGTACCT |
| | | GCGGATCCTCCGACCGCCTTCAACAAGGACAAGCTGAACTCATTCATCACCCA |
| | | GTACTCCACTGGACAGGTCAGCGTGGAGATTGAGTGGGAGCTGCAGAAGGA |
| | | AAACAGCAAGCGCTGGAATCCCGAGATCCAGTACACATCCAACTACTACAAAT |
| | | CTAACAATGTGGAATTTGCTGTGAATACTGAAGGAGTGTATAGTGAGCCTCGC |
| | | CCCATTGGCACCCGTTACCTCACCCGTAATCTGTAA |
| 24 | Pep9hSC5 | ATGGCTGCCGATGGTTATCTTCCAGATTGGCTCGAGGACAACCTCTCTGAAGG |
| | (DNA) | CATTCGCGAGTGGTGGGCTTTGAAACCTGGAGCCCCGCAACCCAAAGCCAAC |
| | | CAGCAACATCAGGACAACGCTCGGGGTCTTGTGCTTCCTGGTTACAAGTACCT |
| | | CGGACCCGGCAACGGACTCGACAAGGGGGAGCCCGTCAACGCGGCGGACGC |
| | | AGCGGCCCTCGAGCACGACAAGGCCTACGACCAGCAGCTCAAGGCGGGTGA |
| | | CAACCCGTACCTCAAGTACAACCACGCCGACGCCGAGTTTCAGGAGCGTCTG |
| | | AAAGAAGATACGTCTTTTGGGGGCAACCTCGGGCGAGCAGTCTTCCAGGCCA |
| | | AGAAGAGGCTTCTCGAACCTCTTGGTCTGGTTGAGGAAGCGGCTAAGACGGC |
| | | TCCTGGAAAGAAGCGTCCGGTAGAGCAGTCTCCACAGGAGCCAGACTCCTCC |
| | | GCGGGCATCGGCAAGTCGGGCGCACAGCCCGCTAAAAAGAGACTCAATTTTG |
| | | GTCAGACTGGCGACACAGAGTCAGTCCCCGACCCTCAACCTATCGGAGAACC |
| | | TCCAGCAGCCCCCTCTGGTGTGGGATCTCTTACAATGGCTTCAGGCGGTGGC |
| | | GCACCAGTCGCAGACAATAACGAAGGCGCCGACGGAGTGGGTAGTTCCTCG |
| | | GGAAATTGGCATTGCGATTCCCAATGGCTGGGCGACAGAGTCATCACCACCA |
| | | GCACCCGAACCTGGGCCCTGCCCACCTACAACAACCACCTCTACAAGCAAATC |
| | | TCCAACAGTACATCAGGAGGAAGCAGCAACGACAACGCCTACTTCGGCTACA |
| | | GCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGAC |
| | | TCAACTTCAAGCTCTTCAACATCCAAGTCAAGGAGGTCACGGACAATAACGGC |
| | | GTCAAGACCATCGCCAATAACCTTACCAGCACGGTTCAAGTGTTTACGGACTC |
| | | GGACTACCAGCTCCCGTACGTCCTCGGCTCTGCGCACGAGGGCTGCCTCCCT |
| | | CCGTTCCCGGCGGACGTCTTCATGATTCCGCAGTACGGCTACCTGACGCTCA |
| | | ACGATGGCAGCCAGGCCGTGGGACGCTCATCCTTTTACTGCCTGGAATACTT |
| | | CCCTTCGCAGATGCTGAGAACGGGCAACAACTTTCAGTTCAGCTACGAGTTTG |
| | | AGAACGTGCCTTTCCACAGCAGCTACGCTCACAGCCAGAGCCTGGACCGGCT |
| | | GATGAATCCTCTCATCGACCAGTACCTGTACTACCTGAGCAAGACTATTAATG |
| | | GATCTGGACAGAATCAACAGACCTTGAAATTTAGCGTGGCCGGACCTAGTAAC |
| | | ATGGCCGTTCAGGGAAGAAACTACATACCTGGACCCTCATACCGGCAGCAGC |
| | | GCGTTTCAACGACAGTGACACAAAACAACAACAGCGAATTTGCCTGGCCTGGT |
| | | GCTTCATCTTGGGCTCTCAATGGCCGTAATTCTCTGATGAATCCTGGCCCGGC |
| | | TATGGCCTCACACAAAGAAGGAGAAGACCGTTTCTTTCCCCTGAGCGGTTCTC |
| | | TCATTTTTGGCAAACAAGGCACAGGAAGAGACAACGTGGATGCGGACAAGGT |
| | | CATGATTACCAACGAAGAGGAAATTAAAACCACTAACCCTGTGGCCACGGAGT |
| | | CCTATGGGCAGGTGGCAACCAACCACCAGGGCCAGAGTCGTGCTGATCGTCC |
| | | TCCTCCTCAGGCGGCCCAAACCGGATGGGTCCAAAACCAGGGAATCTTACCT |
| | | GGCATGGTGTGGCAAGACAGAGACGTGTACCTGCAGGGTCCCATTTGGGCCA |
| | | AGATTCCTCACACGGATGGAAACTTTCACCCGTCTCCGCTGATGGGCGGCTTT |
| | | GGAATGAAACACCCGCCTCCTCAGATCCTCATCAAAAACACGCCTGTACCTGC |
| | | GGATCCTCCGACCGCCTTCAACAAGGACAAGCTGAACTCATTCATCACCCAGT |
| | | ACTCCACTGGACAGGTCAGCGTGGAGATTGAGTGGGAGCTGCAGAAGGAAA |
| | | ACAGCAAGCGCTGGAATCCCGAGATCCAGTACACATCCAACTACTACAAATCT |
| | | AACAATGTGGAATTTGCTGTGAATACTGAAGGAGTGTATAGTGAGCCTCGCCC |
| | | CATTGGCACCCGTTACCTCACCCGTAATCTGTAA |
| 25 | Pep9hSC9 | ATGGCTGCCGATGGTTATCTTCCAGATTGGCTCGAGGACAACCTCTCTGAAGG |
| | (DNA) | CATTCGCGAGTGGTGGGCTTTGAAACCTGGAGCCCCGCAACCCAAAGCCAAC |
| | | CAGCAACATCAGGACAACGCTCGGGGTCTTGTGCTTCCTGGTTACAAGTACCT |
| | | CGGACCCGGCAACGGACTCGACAAGGGGGAGCCCGTCAACGCGGCGGACGC |
| | | AGCGGCCCTCGAGCACGACAAGGCCTACGACCAGCAGCTCAAGGCGGGTGA |
| | | CAACCCGTACCTCAAGTACAACCACGCCGACGCCGAGTTTCAGGAGCGTCTG |
| | | AAAGAAGATACGTCTTTTGGGGGCAACCTCGGGCGAGCAGTCTTCCAGGCCA |
| | | AGAAGAGGCTTCTCGAACCTCTTGGTCTGGTTGAGGAAGCGGCTAAGACGGC |
| | | TCCTGGAAAGAAGCGTCCGGTAGAGCAGTCTCCACAGGAGCCAGACTCCTCC |
| | | GCGGGCATCGGCAAGTCGGGCGCACAGCCCGCTAAAAAGAGACTCAATTTTG |
| | | GTCAGACTGGCGACACAGAGTCAGTCCCCGACCCTCAACCTATCGGAGAACC |
| | | TCCAGCAGCCCCCTCTGGTGTGGGATCTCTTACAATGGCTTCAGGCGGTGGC |
| | | GCACCAGTCGCAGACAATAACGAAGGCGCCGACGGAGTGGGTAGTTCCTCG |
| | | GGAAATTGGCATTGCGATTCCCAATGGCTGGGCGACAGAGTCATCACCACCA |
| | | GCACCCGAACCTGGGCCCTGCCCACCTACAACAACCACCTCTACAAGCAAATC |
| | | TCCAACAGTACATCAGGAGGAAGCAGCAACGACAACGCCTACTTCGGCTACA |
| | | GCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGAC |
| | | TCAACTTCAAGCTCTTCAACATCCAAGTCAAGGAGGTCACGGACAATAACGGC |
| | | GTCAAGACCATCGCCAATAACCTTACCAGCACGGTTCAAGTGTTTACGGACTC |
| | | GGACTACCAGCTCCCGTACGTCCTCGGCTCTGCGCACGAGGGCTGCCTCCCT |
| | | CCGTTCCCGGCGGACGTCTTCATGATTCCGCAGTACGGCTACCTGACGCTCA |
| | | ACGATGGCAGCCAGGCCGTGGGACGCTCATCCTTTTACTGCCTGGAATACTT |
| | | CCCTTCGCAGATGCTGAGAACGGGCAACAACTTTCAGTTCAGCTACGAGTTTG |
| | | AGAACGTGCCTTTCCACAGCAGCTACGCTCACAGCCAGAGCCTGGACCGGCT |
| | | GATGAATCCTCTCATCGACCAGTACCTGTACTACCTGAGCAAGACTATTAATG |
| | | GATCTGGACAGAATCAACAGACCTTGAAATTTAGCGTGGCCGGACCTAGTAAC |
| | | ATGGCCGTTCAGGGAAGAAACTACATACCTGGACCCTCATACCGGCAGCAGC |
| | | GCGTTTCAACGACAGTGACACAAAACAACAACAGCGAATTTGCCTGGCCTGGT |
| | | GCTTCATCTTGGGCTCTCAATGGCCGTAATTCTCTGATGAATCCTGGCCCGGC |
| | | TATGGCCTCACACAAAGAAGGAGAAGACCGTTTCTTTCCCCTGAGCGGTTCTC |
| | | TCATTTTTGGCAAACAAGGCACAGGAAGAGACAACGTGGATGCGGACAAGGT |
| | | CATGATTACCAACGAAGAGGAAATTAAAACCACTAACCCTGTGGCCACGGAGT |
| | | CCTATGGGCAGGTGGCAACCAACCACCAGGGCCAGAGTGCGCGGGGCGACA |
| | | TGCTCGGGAAGGCGGCCCAAACCGGATGGGTCCAAAACCAGGGAATCTTACC |
| | | TGGCATGGTGTGGCAAGACAGAGACGTGTACCTGCAGGGTCCCATTTGGGCC |
| | | AAGATTCCTCACACGGATGGAAACTTTCACCCGTCTCCGCTGATGGGCGGCTT |
| | | TGGAATGAAACACCCGCCTCCTCAGATCCTCATCAAAAACACGCCTGTACCTG |
| | | CGGATCCTCCGACCGCCTTCAACAAGGACAAGCTGAACTCATTCATCACCCAG |
| | | TACTCCACTGGACAGGTCAGCGTGGAGATTGAGTGGGAGCTGCAGAAGGAAA |
| | | ACAGCAAGCGCTGGAATCCCGAGATCCAGTACACATCCAACTACTACAAATCT |
| | | AACAATGTGGAATTTGCTGTGAATACTGAAGGAGTGTATAGTGAGCCTCGCCC |
| | | CATTGGCACCCGTTACCTCACCCGTAATCTGTAA |
| 26 | Shuffle32 | ATGGCTGCCGATGGTTATCTTCCAGATTGGCTCGAGGACAACCTTTCTGAAGG |
| | (DNA) | CATTCGCGAGTGGTGGGACCTGAAACCTGGAGCCCCCAAGCCCAAGGCCAAC |
| | | CAGCAGAAGCAGGACGACGGCCGGGGTCTGGTGCTTCCTGGCTACAAGTAC |
| | | CTCGGACCCTTCAACGGACTCGACAAGGGAGAGCCGGTCAACGAGGCAGAC |
| | | GCCGCGGCCCTCGAGCACGACAAGGCCTACGACCAGCAGCTCAAAGCGGGT |
| | | GACAATCCGTACCTGCGGTATAACCACGCCGACGCCGAGTTTCAGGAGCGTC |
| | | TGCAAGAAGATACGTCTTTTGGGGGCAACCTCGGGCGAGCAGTCTTCCAGGC |
| | | CAAAAAGAGGCTTCTTGAACCTCTTGGTCTGGTTGAGGAAGCGGCTAAGACG |
| | | GCTCCTGCAAAGAAGAGACCGGTAGAGCCGTCACCTCAGCGTTCCCCCGACT |
| | | CCTCCACGGGCATCGGCAAGAAAGGCCAGCAGCCCGCCAGAAAGAGACTCA |
| | | ATTTCGGTCAGACTGGCGACTCAGAGTCAGTTCCAGACCCACAACCTCTCGGA |
| | | GAACCTCCAGCAACCCCCGCTGCTGTGGGACCTACTACAATGGCTTCAGGCG |
| | | GTGGCGCACCAATGGCAGACAATAACGAAGGCGCCGACGGAGTGGGTAATG |
| | | CCTCAGGAAATTGGCATTGCGATTCCACATGGCTGGGCGACAGAGTCATCAC |
| | | CACCAGCACCAGAACCTGGGCCCTGCCCACTTACAACAACCACCTCTACAAAC |
| | | AAATTTCCAGCCAATCAGGAGCCTCGAACGACAATCACTACTTTGGCTACAGC |
| | | ACCCCTTGGGGGTATTTTGACTTCAACAGATTCCACTGCCACTTTTCACCACGT |
| | | GACTGGCAGCGACTCATCAACAACAACTGGGGATTCCGACCCAAGAGACTCA |
| | | ACTTCAAGCTCTTCAACATCCAAGTCAAGGAGGTCACGACAAATGATGGCGTC |
| | | ACGACCATCGCTAATAACCTTACCAGCACGGTTCAAGTCTTCTCGGACTCGGA |
| | | GTACCAGTTGCCGTACGTCCTCGGCTCTGCGCACCAGGGCTGCCTCCCTCCG |
| | | TTCCCGGCGGACGTGTTCATGATTCCGCAGTACGGCTACCTAACGCTCAACAA |
| | | CGGTAGTCAGGCCGTGGGACGCTCCTCCTTCTACTGCCTGGAATACTTTCCTT |
| | | CGCAGATGCTGAGAACGGGCAATAACTTTACCTTCAGCTACACCTTCGAGGAC |
| | | GTGCCTTTCCACAGCAGCTACGCGCACAGCCAGAGCCTGGACCGGCTGATGA |
| | | ACCCTCTCATCGACCAGTACCTGTATTACTTGAGCAGAACAAACACTCCAAGT |
| | | GGAACCACCACGCAGTCAAGGCTTCAGTTTTCTCAGGCCGGAGCGAGTGACA |
| | | TTCGGGACCAGTCTAGGAACTGGCTTCCTGGACCCTGTTACCGCCAGCAGCG |
| | | AGTATCAAAGACATCTGCGGATAACAACAACAGTGAATACTCGTGGACTGGA |
| | | GCTACCAAGTACCACCTCAATGGCAGAGACTCTCTGGTGAATCCGGGCCCGG |
| | | CCATGGCGAGCCACAAGGACGATGAAGAAAAGTTTTTTCCTCAGAGCGGGGT |
| | | TCTCATCTTTGGGAAGCAAGGCTCAGAGAAAACAAATGTGGACATTGAAAAG |
| | | GTCATGATTACAGACGAAGAGGAAATCAGGACAACCAATCCCGTGGCTACGG |
| | | AGCAGTATGGTTCTGTATCTACCAACCTCCAGAGAGGCAACAGACAAGCAGCT |
| | | ACCGCAGATGTCAACACACAAGGCGTTCTTCCAGGCATGGTCTGGCAGGACA |
| | | GAGATGTGTACCTTCAGGGGCCCATCTGGGCAAAGATTCCACACACGGACGG |
| | | ACATTTTCACCCCTCTCCCCTCATGGGTGGATTCGGACTTAAACACCCTCCTCC |
| | | ACAGATTCTCATCAAGAACACCCCGGTACCTGCGAATCCTTCGACCACCTTCA |
| | | GTGCGGCAAAGTTTGCTTCCTTCATCACACAGTACTCCACGGGACAGGTCAGC |
| | | GTGGAAATCGAGTGGGAGCTGCAGAAGGAGAACAGCAAACGCTGGAACCCA |
| | | GAGATTCAGTACACTTCAAACTACTACAAATCTACAAATGTGGACTTTGCCGTT |
| | | GACAGCCAGGGTGTTTACTCTGAGCCTCGCCCTATTGGCACTCGTTACCTCAC |
| | | CCGTAATCTG |
| 27 | Shuffle35 | ATGGCTGCCGATGGTTATCTTCCAGATTGGCTCGAGGACAACCTCTCTGAAGG |
| | (DNA) | AATAAGACAGTGGTGGAAGCTCAAACCTGGCCCACCACCACCAAAGCCCGCA |
| | | GAGCGGCATAAGGACGACAGCAGGGGTCTTGTGCTTCCTGGGTACAAGTACC |
| | | TCGGACCCTTCAACGGACTCGACAAGGGAGAGCCGGTCAACGAGGCAGACG |
| | | CCGCGGCCCTCGAGCACGACAAGGCCTACGACCAGCAGCTCAAAGCGGGTG |
| | | ACAATCCGTACCTGCGGTATAACCACGCCGACGCCGAGTTTCAGGAGCGTCT |
| | | GCAAGAAGATACGTCTTTTGGGGGCAACCTCGGGCGAGCAGTCTTCCAGGCC |
| | | AAGAAGCGGGTTCTCGAACCTCTCGGTCTGGTTGAGGAAGGCGCTAAGACGG |
| | | CTCCTGGAAAGAAGAGGCCTGTAGAGCAGTCTCCTCAGGAACCGGACTCCTC |
| | | CGCGGGTATTGGCAAATCGGGTGCACAGCCCGCTAAAAAGAGACTCAATTTC |
| | | GGTCAGACTGGCGACTCAGAGTCAGTCCCCGACCCACAACCTCTCGGAGAAC |
| | | CTCCAGCAACCCCCGCTGCTGTGGGACCTACTACAATGGCTTCAGGCGGTGG |
| | | CGCACCAATGGCAGACAATAACGAAGGCGCCGACGGAGTGGGTAATGCCTCA |
| | | GGAAATTGGCATTGCGATTCCACATGGCTGGGCGACAGAGTCATCACCACCA |
| | | GCACCAGAACCTGGGCCCTGCCCACTTACAACAACCATCTCTACAAGCAAATC |
| | | TCCAGTGCTTCAACGGGGGCCAGCAACGACAACCACTACTTCGGCTACAGCA |
| | | CCCCCTGGGGGTATTTTGACTTTAACAGATTCCACTGCCATTTCTCACCACGT |
| | | GACTGGCAGCGACTCATCAACAACAATGGGGGATTCCGGCCCAAGAGACTCA |
| | | ACTTCAAGCTCTTCAACATCCAAGTCAAGGAGGTCACGACGAATGATGGCGTC |
| | | ACGACCATCGCTAATAACCTTACCAGCACGGTTCAAGTCTTCTCGGACTCGGA |
| | | GTACCAGTTGCCGTACGTCCTCGGCTCTGCGCACCAGGGCTGCCTCCCTCCG |
| | | TTCCCGGCGGACGTGTTCATGATTCCGCAGTACGGCTACCTAACGCTCAACAA |
| | | TGGCAGCCAGGCAGTGGGACGGTCATCCTTTTACTGCCTGGAATATTTCCCGT |
| | | CGCAGATGCTGCGTACCGGAAACAACTTTACCTTCAGCTACACTTTTGAGGAC |
| | | GTTCCTTTCCACAGCAGCTACGCTCACAGCCAGAGTCTGGACCGTCTCATGAA |
| | | TCCTCTCATCGACCAGTACCTGTATTACTTGAGCAGAACAAACACTCCAAGTG |
| | | GAACCACCACGCAGTCAAGGCTTCAGTTTTCTCAGGCCGGAGCGAGTGACAT |
| | | TCGGGACCAGTCTAGGAACTGGCTTCCTGGACCCTGTTACCGCCAGCAGCGA |
| | | GTATCAAAGACATCTGCGGATAACAACAACAGTGAATACTCGTGGACTGGAG |
| | | CTACCAAGTACCACCTCAATGGCAGAGACTCTCTGGTGAATCCGGGCCCGGC |
| | | CATGGCAAGCCACAAGGACGATGAAGAAAAGTTTTTTCCTCAGAGCGGGGTT |
| | | CTCATCTTTGGGAAGCAAGGCTCAGAGAAAACAAATGTGGACATTGAAAAGG |
| | | TCATGATTACAGACGAAGAGGAAATCAGGACAACCAATCCCGTGGCTACGGA |
| | | GCAGTATGGTTCTGTATCTACCAACCTCCAGAGAGGCAACAGACAAGCAGCTA |
| | | CCGCAGATGTCAACACACAAGGCGTTCTTCCAGGCATGGTCTGGCAGGACAG |
| | | AGATGTGTACCTTCAGGGGCCCATCTGGGCAAAGATTCCACACACGGATGGA |
| | | CACTTTCATCCTTCTCCTCTGATGGGAGGCTTTGGACTGAAACATCCGCCTCC |
| | | TCAAATCATGATCAAAAATACTCCGGTACCGGCAAATCCTTCGACCACCTTCA |
| | | GTGCGGCAAAGTTTGCTTCATTTATCACTCAGTACTCCACTGGACAGGTCAGC |
| | | GTGGAAATTGAGTGGGAGCTACAGAAAGAAAACAGCAAACGTTGGAATCCAG |
| | | AGATTCAGTACACTTCCAACTACTACAAATCTACAAGTGTGGACTTTGCTGTTA |
| | | ATACAGAAGGCGTGTACTCTGAACCCCGCCCCATTGGCACCAGATACCTGACT |
| | | CGTAATCTG |

1. An AAV capsid polypeptide capable of increasing the transduction efficiency of a cell of the peripheral nervous system of an AAV vector comprising the capsid polypeptide compared to an AAV vector comprising the AAV2 capsid of SEQ ID NO:1 or the AAV9 capsid of SEQ ID NO:2, wherein the capsid polypeptide comprises:
(i) a 12 amino acid substitution at positions 585-589 of SEQ ID NO:1 with a sequence comprising or consisting of the amino acid sequence set forth in SEQ ID NO:3; or
(ii) a 12 amino acid substitution at positions 586-590 of SEQ ID NO:2 with a sequence comprising or consisting of the amino acid sequence set forth in SEQ ID NO:8 or 9.

2. The AAV capsid polypeptide of claim 1, wherein the cell of the peripheral nervous system is a Schwann cell.

3. The AAV capsid polypeptide of claim 1, comprising:
a) relative to the AAV2 capsid polypeptide set forth in SEQ ID NO:1, a 12 amino acid substitution at positions 585-589 of SEQ ID NO:1 with a sequence comprising or consisting of the amino acid sequence set forth in SEQ ID NO:3, and wherein the capsid polypeptide comprises at least about 80% sequence identity to positions 1-584 and/or 590-735 of SEQ ID NO:1, flanking the amino acid substitution; or
b) relative to the AAV9 capsid polypeptide set forth in SEQ ID NO:2, a 12 amino acid substitution at positions 586-590 of SEQ ID NO:2 with a sequence comprising or consisting of the amino acid sequence set forth in SEQ ID NO:8 or 9, and wherein the capsid polypeptide comprises at least about 80% sequence identity to positions 1-585 and/or 591-736 of SEQ ID NO:2, flanking the amino acid substitution.

4. The AAV capsid polypeptide of claim 3, wherein the amino acid substitution of a comprises or consists of the amino acid sequence set forth in SEQ ID NO:4 or 5 or a sequence having at least about 85% sequence identity thereto.

5. The AAV capsid polypeptide of claim 4, wherein the capsid polypeptide comprises or consists of the amino acid sequence of SEQ ID NO:1 from positions 1-584 and 590-735 of SEQ ID NO:1, flanking the amino acid substitution.

6. The AAV capsid polypeptide of claim 3, comprising the amino acid sequence set forth in SEQ ID NO:6 or 7, or a sequence having at least about 80% sequence identity thereto.

7. (canceled)

8. The AAV capsid polypeptide of claim 3, wherein the amino acid substitution of b) comprises or consists of the amino acid sequence set forth in any one of SEQ ID NOs:10-13 or a sequence having at least about 85% sequence identity thereto.

9. The AAV capsid polypeptide of claim 8, wherein the capsid polypeptide comprises or consists of the amino acid sequence of SEQ ID NO:2 from positions 1-585 and 591-736 of SEQ ID NO:2, flanking the amino acid substitution.

10. The AAV capsid polypeptide of claim 9, comprising the amino acid sequence set forth in any one of SEQ ID NOs:14-17, or a sequence having at least about 80% sequence identity thereto.

11. An AAV capsid polypeptide, comprising:
a) a peptide modification relative to the AAV2 capsid polypeptide set forth in SEQ ID NO:1, wherein the peptide modification comprises a substitution at amino acid positions 585-589 of SEQ ID NO:1 with a sequence comprising or consisting of the amino acid sequence set forth in SEQ ID NO:3, and wherein the portion of the capsid polypeptide that is not the peptide modification comprises at least about 80% sequence identity to positions 1-584 and/or 590-735 of SEQ ID NO:1; or
b) a peptide modification relative to the AAV9 capsid polypeptide set forth in SEQ ID NO:2, wherein the peptide modification comprises a substitution at amino acid positions 586-590 of SEQ ID NO:2 with a sequence comprising or consisting of the amino acid sequence set forth in SEQ ID NO:8 or 9, and wherein the portion of the capsid polypeptide that is not the peptide modification comprises at least about 80% sequence identity to positions 1-585 and/or 591-736 of SEQ ID NO:2.

12. The AAV capsid polypeptide of claim 11, wherein the peptide modification of a comprises or consists of the amino acid sequence set forth in SEQ ID NO:4 or 5 or a sequence having at least about 85% sequence identity thereto.

13-16. (canceled)

17. The AAV capsid polypeptide of claim 11, wherein the peptide modification of b) comprises or consists of the amino acid sequence set forth in any one of SEQ ID NOs:10-13 or a sequence having at least about 85% sequence identity thereto.

18-20. (canceled)

21. An AAV capsid polypeptide comprising an amino sequence as set forth in SEQ ID NO:18 or 19, or a sequence having at least about 80% sequence identity thereto.

22. An AAV vector, comprising the capsid polypeptide of claim 1.

23-25. (canceled)

26. The AAV vector of claim 22, further comprising a heterologous coding sequence.

27-28. (canceled)

29. An isolated nucleic acid molecule encoding the capsid polypeptide of claim 1.

30-32. (canceled)

33. A method for introducing a heterologous coding sequence into a host cell, comprising contacting a host cell with the AAV vector of claim 26.

34. The method of claim 33, wherein the host cell is a cell of the peripheral nervous system.

35. (canceled)

36. The method of claim 33, wherein contacting the host cell with the AAV vector comprises administering the AAV vector to a subject to effect treatment of a disease or condition of the peripheral nervous system.

37-39. (canceled)

40. A method for producing an AAV vector, comprising culturing a host cell comprising a nucleic acid molecule encoding the capsid polypeptide of claim 1, an AAV rep gene, a heterologous coding sequence flanked by AAV inverted terminal repeats, and helper functions for generating a productive AAV infection, under conditions suitable to facilitate assembly of an AAV vector comprising a capsid comprising the capsid polypeptide of claim 1, wherein the capsid encapsidates the heterologous coding sequence.

41-42. (canceled)
